(19) United States
(12) Patent Application Publication
(10) Pub. No.: US 2012/0053165 A1
(43) Pub. Date: Mar. 1, 2012

(54) Novel Phenyl Imidazoles and Phenyl Triazoles As Gamma-Secretase Modulators

(75) Inventors: Martin Patrick Allen, Groton, CT (US); Christopher William Am Ende, Groton, CT (US); Michael Aaron Brodney, Groton, CT (US); Amy Beth Dounay, Groton, CT (US); Douglas Scott Johnson, Groton, CT (US); Martin Youngjin Pettersson, Groton, CT (US); Jacob Bradley Schwartz, Groton, CT (US); Tuan Phong Tran, Groton, CT (US)

(73) Assignee: Pfizer Inc., New York, NY (US)

(21) Appl. No.: 13/203,988

(22) PCT Filed: Mar. 2, 2010
(86) PCT No.: PCT/IB2010/050896

§ 371 Date: Nov. 14, 2011

Related U.S. Application Data
(60) Provisional application No. 61/156,963, filed on Mar. 3, 2009.

(51) Int. Cl. A61K 31/4178; A61K 31/4164; C07D 413/12; A61K 31/422; A61K 31/4245; C07D 249/08; A61K 31/4196; C07D 405/12; C07D 409/12; C07D 403/12; C07D 417/12; A61K 31/427; A61K 31/423; C07D 403/10; C07D 401/10; A61K 31/454; A61K 31/5377; A61K 31/437; A61P 25/00; A61P 25/18; C07D 233/61

(52) U.S. Cl. 514/210.18; 548/343.5; 514/399; 548/247; 514/378; 548/143; 514/364; 548/269.4; 514/383; 548/311.1; 514/397; 548/311.4; 548/312.4; 548/314.7; 548/312.1; 548/204; 514/365; 548/241; 514/379; 546/210; 514/326; 544/139; 514/235.8; 514/292

(57) ABSTRACT

Compounds and pharmaceutically acceptable salts of the compounds are disclosed, wherein the compounds have the structure of Formula (I) as defined in the specification. Corresponding pharmaceutical compositions, methods of treatment, methods of synthesis, and intermediates are also disclosed.

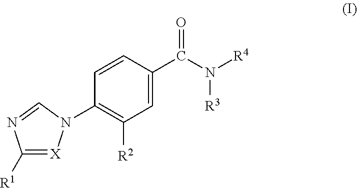

### FIELD OF THE INVENTION

The present invention relates to the treatment of Alzheimer's disease and other neurodegenerative and/or neurological disorders in mammals, including humans. This invention also relates to the modulation, in mammals, including humans, of the production of A-beta peptides that can contribute to the formation of neurological deposits of amyloid protein. More particularly, this invention relates to phenyl imidazole and phenyl triazole compounds useful for the treatment of neurodegenerative and/or neurological disorders, such as Alzheimer's disease and Down's Syndrome, related to A-beta peptide production.

### BACKGROUND OF THE INVENTION

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner et al., J. Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Several programs have been advanced by research groups to ameliorate the pathological processes causing dementia, AD, CM and prion-mediated diseases. γ-Secretase modulators are one such strategy and numerous compounds are under evaluation by pharmaceutical groups. The present invention relates to a group of brain penetrable γ-secretase modulators and as such are useful as γ-secretase modulators for the treatment of AD (see Ann. Rep. Med. Chem. 2007, Olsen et al., 42: 27-47).

### SUMMARY OF THE INVENTION

The present invention is directed to a compound, including the pharmaceutically acceptable salts thereof, having the structure of formula Ia:

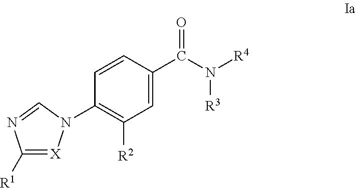

wherein

X is CH or N;

R¹ is hydrogen, C₁₋₆alkyl, C₃₋₆cycloalkyl, or C₂₋₆alkenyl, wherein said alkyl, cycloalkyl and alkenyl is optionally substituted with one to three halogen or —(CH₂)ₜ—C₃₋₆cycloalkyl;

R² is hydrogen, —CF₃, cyano, halogen, C₁₋₆alkyl, or —OR⁵;

R³ and R⁴ are each independently hydrogen, C₁₋₆alkyl, C₂₋₆alkenyl, —(CH₂)ₜ-cycloalkyl, —(CH₂)ₜ-heterocycloalkyl, —(CH₂)ₜ-aryl, or —(CH₂)ₜ-heteroaryl; wherein said alkyl, alkenyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl R³ or R⁴ substituent is optionally independently substituted with one to three R⁶; or R³ and R⁴ together with the nitrogen they are bonded to form a heterocycloalkyl moiety, wherein said heterocycloalkyl moiety is optionally independently substituted with one to three R⁶;

R⁵ is hydrogen, C₁₋₆alkyl, C₃₋₆cycloalkyl, C₂₋₆alkenyl, or C₂₋₆alkynyl, wherein said alkyl, cycloalkyl, alkenyl, and alkynyl is optionally substituted with cyano, or one to three halogen;

each R⁶ is independently hydrogen, halogen, —CF₃, C₂₋₆alkylidene, —(CH₂)ₜ-cycloalkyl, —(CH₂)ₜ-heterocycloalkyl, —(CH₂)ₜ-aryl, or —(CH₂)ₜ-heteroaryl, —(CH₂)ₜ—OR⁷, —C(O)R⁷, —CN, or —N(R⁷)₂, wherein said —(CH₂)ₜ, —(CH₂)ₜ-cycloalkyl, —(CH₂)ₜ-aryl, —(CH₂)ₜ-heterocycloalkyl and —(CH₂)ₜ-heteroaryl substituent is optionally independently substituted with one to three alkyl, halogen, —CF₃ or —OR⁷;

each R⁷ is independently hydrogen, C₁₋₆alkyl, —CF₃, —SO₂R⁵, —N(R⁸)₂, —(CH₂)ₜ-cycloalkyl, —(CH₂)ₜ-heterocycloalkyl, —(CH₂)ₜ-aryl, or —(CH₂)ₜ-heteroaryl wherein said alkyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl is optionally substituted with C₁₋₆alkyl, C₂₋₆alkenyl, C₂₋₆alkynyl, halogen, —CF₃, or —OCF₃;

each R⁸ is hydrogen, C₁₋₆alkyl, or —(CH₂)ₜ-aryl, wherein said C₁₋₆alkyl or —(CH₂)ₜ-aryl is optionally substituted with one to three halogen; and

each t is an integer independently selected from 0, 1, 2, 3, and 4.

The present invention is also directed to a compound, including the pharmaceutically acceptable salts thereof, having the structure of formula I:

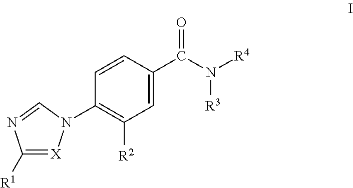

wherein

X is CH or N;

R¹ is hydrogen, C₁₋₆alkyl, C₃₋₆cycloalkyl, or C₂₋₆alkenyl, wherein said alkyl, cycloalkyl and alkenyl is optionally substituted with one to three halogen;

R² is hydrogen, —CF₃, cyano, halogen, C₁₋₆alkyl, or —OR⁵;

R³ and R⁴ are each independently hydrogen, C₁₋₆alkyl, C₂₋₆alkenyl, —(CH₂)ₜ-cycloalkyl, —(CH₂)ₜ-heterocycloalkyl, —(CH₂)ₜ-aryl, or —(CH₂)ₜ-heteroaryl; wherein said alkyl, alkenyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl R³ or R⁴ substituent is optionally independently substituted with one to three R⁶; or R³ and R⁴ together with the nitrogen they are bonded to form a heterocycloalkyl moiety, wherein said heterocycloalkyl moiety is optionally independently substituted with one to three R⁶;

R⁵ is hydrogen, C₁₋₆alkyl, C₃₋₆cycloalkyl, C₂₋₆alkenyl, or C₂₋₆alkynyl, wherein said alkyl, cycloalkyl, alkenyl, and alkynyl is optionally substituted with cyano, or one to three halogen;

each R⁶ is independently hydrogen, halogen, —CF₃, C₁₋₆alkyl, —(CH₂)ₜ-cycloalkyl, —(CH₂)ₜ-heterocycloalkyl, —(CH₂)ₜ-aryl, or —(CH₂)ₜ-heteroaryl, —OR⁷, —C(O)R⁷, —CN, or —N(R⁷)₂, wherein said —(CH₂)ₜ, —(CH₂)ₜ-cycloalkyl, —(CH₂)ₜ-aryl, —(CH₂)ₜ-heterocycloalkyl and —(CH₂)ₜ-heteroaryl substituent is optionally independently substituted with one to three alkyl, halogen, —CF₃ or —OR⁷;

each R⁷ is independently hydrogen, C₁₋₆alkyl, —CF₃, —SO₂R⁸, —N(R⁸)₂, —(CH₂)ₜ-cycloalkyl, —(CH₂)ₜ-heterocycloalkyl, —(CH₂)ₜ-aryl, or —(CH₂)ₜ-heteroaryl wherein said alkyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl is optionally substituted with C₁₋₆alkyl, C₂₋₆alkenyl, C₂₋₆alkynyl, halogen, —CF₃, or —OCF₃;

each R⁸ is hydrogen, C₁₋₆alkyl, or —(CH₂)ₜ-aryl, wherein said C₁₋₆alkyl or —(CH₂)ₜ-aryl is optionally substituted with one to three halogen; and

each t is an integer independently selected from 0, 1, 2, 3, and 4.

In one embodiment of the invention, X is CH.

In another embodiment of the invention, X is N.

In one embodiment of the invention R³ and R⁴ together with the nitrogen they are bonded to form a heterocycloalkyl moiety, wherein said heterocycloalkyl moiety is optionally independently substituted with one to three R⁶.

In one embodiment of the invention R³ and R⁴ together with the nitrogen they are bonded to form a heterocycloalkyl, wherein said heterocycloalkyl is optionally independently substituted with one R⁶. In another embodiment of the invention, R⁶ is —OR⁷ or —(CH₂)ₜ-aryl. In yet another embodiment of the invention, the heterocycloalkyl is optionally substituted with one R⁶ wherein R⁶ is —OR⁷ and R⁷ is —(CH₂)ₜ-aryl optionally substituted with C₁₋₆alkyl, C₂₋₆alkenyl, C₂₋₆alkynyl, halogen, —CF₃, or —OCF₃.

In another embodiment of the invention, the heterocycloalkyl is optionally substituted with one R⁶ wherein R⁶ is —(CH₂)ₜ-aryl, wherein t is 0 and said aryl is optionally substituted with C₁₋₆alkyl, C₂₋₆alkenyl, C₂₋₆alkynyl, halogen, —CF₃, or —OCF₃.

In one embodiment of the invention R³ and R⁴ together with the nitrogen they are bonded to form a heterocycloalkyl, wherein said heterocycloalkyl is optionally substituted with two R⁶.

In one embodiment of the invention R³ and R⁴ together with the nitrogen they are bonded to form a heterocycloalkyl, wherein said heterocycloalkyl is optionally substituted with three R⁶.

In one embodiment of the invention R⁴ is C₁₋₆alkyl, —(CH₂)ₜ-cycloalkyl, —(CH₂)ₜ-heterocycloalkyl, —(CH₂)ₜ-aryl, or —(CH₂)ₜ-heteroaryl, wherein said C₁₋₆alkyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl R⁴ substituent is optionally substituted with one to three R⁶.

In one embodiment of the invention R⁴ is C₁₋₆alkyl wherein said C₁₋₆alkyl R⁴ substituent is optionally substituted with one to three R⁶.

In one embodiment of the invention R⁴ is —(CH₂)ₜ-cycloalkyl wherein said —(CH₂)ₜ-cycloalkyl R⁴ substituent is optionally substituted with one to three R⁶. In one embodiment of the invention R⁴ is —(CH₂)ₜ-cycloalkyl wherein said —(CH₂)ₜ-cycloalkyl R⁴ substituent is optionally substituted with one R⁶, and R⁶ is halogen or —CN. In one embodiment of the invention R⁴ is —(CH₂)ₜ-cycloalkyl wherein said —(CH₂)ₜ-cycloalkyl R⁴ substituent is optionally substituted with two R⁶, and each R⁶ is independently hydrogen, halogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, —C(O)R⁷, —CN, or —N(R⁷)₂. In another embodiment of the invention R⁴ is —(CH₂)ₜ-cycloalkyl wherein said —(CH₂)ₜ-cycloalkyl R⁴ substituent is optionally substituted with three R⁶, and each R⁶ is independently hydrogen, halogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, —C(O)R⁷, —CN, or —N(R⁷)₂. Other embodiments of interest to the present inventors are those compounds additionally wherein R³ is hydrogen.

In one embodiment of the invention R⁴ is —(CH₂)ₜ-heterocycloalkyl wherein said —(CH₂)ₜ-heterocycloalkyl R⁴ substituent is optionally substituted with one to three R⁶. In one embodiment of the invention R⁴ is —(CH₂)ₜ-heterocycloalkyl wherein said —(CH₂)ₜ-heterocycloalkyl R⁴ substituent is optionally substituted with one R⁶, and R⁶ is halogen or —CN. In one embodiment of the invention R⁴ is —(CH₂)ₜ-heterocycloalkyl wherein said —(CH₂)ₜ-heterocycloalkyl R⁴ substituent is optionally substituted with two R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. In another embodiment of the invention R⁴ is —(CH₂)ₜ-heterocycloalkyl wherein said —(CH₂)ₜ-heterocycloalkyl R⁴ substituent is optionally substituted with three R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. Other embodiments of interest to the present inventors are those compounds additionally wherein R³ is hydrogen.

In one embodiment of the invention R⁴ is —(CH₂)ₜ-aryl wherein said —(CH₂)ₜ-aryl R⁴ substituent is optionally substituted with one to three R⁶. In one embodiment of the invention R⁴ is —(CH₂)ₜ-aryl wherein said —(CH₂)ₜ-aryl R⁴ substituent is optionally substituted with one R⁶, and R⁶ is halogen or —CN. In one embodiment of the invention R⁴ is —(CH₂)ₜ-aryl wherein said —(CH₂)ₜ-aryl R⁴ substituent is optionally substituted with two R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. In one embodiment of the invention R⁴ is —(CH₂)ₜ-aryl wherein said —(CH₂)ₜ-aryl R⁴ substituent is optionally substituted with three R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. Other embodiments of interest to the present inventors are those compounds additionally wherein R³ is hydrogen.

In one embodiment of the invention R⁴ is —(CH₂)ₜ-heteroaryl wherein said —(CH₂)ₜ-heteroaryl R⁴ substituent is optionally substituted with one to three R⁶. In one embodiment of the invention R⁴ is —(CH₂)ₜ-heteroaryl wherein said —(CH₂)ₜ-heteroaryl R⁴ substituent is optionally substituted with one R⁶, and R⁶ is halogen or —CN. In one embodiment of the invention R⁴ is —(CH₂)ₜ-heteroaryl wherein said —(CH₂)ₜ-heteroaryl R⁴ substituent is optionally substituted with two R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. In one embodiment of the invention R⁴ is —(CH₂)ₜ-heteroaryl wherein said —(CH₂)ₜ-heteroaryl R⁴ substituent is optionally substituted with three R⁶, and each R⁶ is independently hydrogen, C₁₋₆alkyl, —CF₃, —(CH₂)ₜ-cycloalkyl, —(CH₂)ₜ-heterocycloalkyl, —(CH₂)ₜ-aryl, —(CH₂)ₜ-heteroaryl, halogen, —OR⁷, —C(O)R⁷, —CN, or —N(R⁷)₂. Other embodiments of interest to the present inventors are those compounds additionally wherein R³ is hydrogen.

In one embodiment of the invention R¹ is C₁₋₆alkyl. In an example of this embodiment, R¹ is methyl.

In one embodiment of the invention R² is halogen.

In one embodiment of the invention R² is —OR⁵. In one embodiment of the invention, R⁵ is hydrogen or C₁₋₆alkyl. In one embodiment of the invention R⁵ is hydrogen. In another embodiment of the invention R⁵ is C₁₋₆alkyl. In an example of this embodiment, R⁵ is methyl.

In one embodiment of the invention R³ is hydrogen, C₁₋₆alkyl, or —(CH₂)ₜ-cycloalkyl wherein said alkyl or —(CH₂)ₜ-cycloalkyl is optionally independently substituted with one to three halogen.

In one embodiment of the invention R³ is hydrogen.

In one embodiment of the invention R³ is C₁₋₆alkyl. In one embodiment of the invention, R³ is methyl.

In one embodiment of the invention t is 0.

In another embodiment of the invention t is 1.

In another embodiment of the invention t is 2.

In another embodiment of the invention t is 3.

In yet another embodiment of the invention t is 4.

It will be understood that the compounds of formula I, and pharmaceutically acceptable salts thereof, also include hydrates, solvates and polymorphs of said compounds of formula I, and pharmaceutically acceptable salts thereof, as discussed below.

In one embodiment, the invention also relates to each of the individual compounds described as Examples 1-140 in the Examples section of the subject application, (including the free bases or pharmaceutically acceptable salts thereof).

In another embodiment the invention relates to a compound selected from the group consisting of:
N-[(5-chloro-1-benzothien-3-yl)methyl]-3-methoxy-4-(4-methyl-1H-imidazol-1-yl)benzamide;
1-[2-methoxy-4-({(3R)-3-[2-(trifluoromethyl)phenoxy]pyrrolidin-1-yl}carbonyl)phenyl]-4-methyl-1H-imidazole;
N-[2-(5-chloro-1H-indol-3-yl)ethyl]-3-methoxy-4-(4-methyl-1H-imidazol-1-yl)benzamide;
3-methoxy-4-(4-methyl-1H-imidazol-1-yl)-N-[(1-phenylcyclopentyl)methyl]benzamide;
3-methoxy-4-(4-methyl-1H-imidazol-1-yl)-N-({4-[4-(trifluoromethyl)phenyl]tetrahydro-2H-pyran-4-yl}methyl)benzamide;
N-(adamantan-2-ylmethyl)-3-methoxy-4-(4-methyl-1H-imidazol-1-yl)benzamide;
N-{[5-(4-chlorophenyl)-2-thienyl]methyl}-3-methoxy-4-(4-methyl-1H-imidazol-1-yl)benzamide;
3-methoxy-4-(4-methyl-1H-imidazol-1-yl)-N-({2-[4-(trifluoromethyl)phenyl]-1,3-thiazol-4-yl}methyl)benzamide;
3-methoxy-4-(4-methyl-1H-imidazol-1-yl)-N-{3-methyl-4-[3-(trifluoromethyl)phenyl]-1H-pyrazol-5-yl}benzamide;
3-methoxy-4-(4-methyl-1H-imidazol-1-yl)-N-[(2-phenyl-1,3-thiazol-5-yl)methyl]benzamide;
N-[2-(5,7-dichloro-2-methyl-1H-indol-3-yl)ethyl]-3-methoxy-4-(4-methyl-1H-imidazol-1-yl)benzamide;
N-{[1-(4-chlorophenyl)cyclopentyl]methyl}-3-methoxy-4-(4-methyl-1H-imidazol-1-yl)benzamide;
N-{[1-(4-bromophenyl)cyclopropyl]methyl}-3-methoxy-4-(4-methyl-1H-imidazol-1-yl)benzamide;
N-{[1-(4-fluorophenyl)cyclobutyl]methyl}-3-methoxy-4-(4-methyl-1H-imidazol-1-yl)benzamide;
1-[2-methoxy-4-({(3S)-3-[2-(trifluoromethyl)phenoxy]pyrrolidin-1-yl}carbonyl)phenyl]-4-methyl-1H-imidazole;
N-[2-(5-bromo-1H-indol-3-yl)ethyl]-3-methoxy-4-(4-methyl-1H-imidazol-1-yl)benzamide;
3-methoxy-4-(4-methyl-1H-imidazol-1-yl)-N-({1-[3-(trifluoromethyl)phenyl]cyclobutyl}methyl)benzamide;
N-{[2-(4-chlorophenyl)-1,3-thiazol-4-yl]methyl}-3-methoxy-4-(4-methyl-1H-imidazol-1-yl)benzamide;
N-(3-chloro-4-fluorobenzyl)-3-methoxy-4-(4-methyl-1H-imidazol-1-yl)benzamide;
N-(5-isopropyl-4-methoxy-2-methylbenzyl)-3-methoxy-4-(4-methyl-1H-imidazol-1-yl)benzamide;
N-(4-ethoxy-5-isopropyl-2-methylbenzyl)-3-methoxy-4-(4-methyl-1H-imidazol-1-yl)benzamide;
N-(5-isopropyl-4-methoxy-2-methylphenethyl)-3-methoxy-4-(4-methyl-1H-imidazol-1-yl)benzamide;
N-(4-ethoxy-5-isopropyl-2-methylphenethyl)-3-methoxy-4-(4-methyl-1H-imidazol-1-yl)benzamide;
N-(1-(5-isopropyl-4-methoxy-2-methylphenyl)propan-2-yl)-3-methoxy-4-(4-methyl-1H-imidazol-1-yl)benzamide;
N-(1-(4-ethoxy-5-isopropyl-2-methylphenyl)propan-2-yl)-3-methoxy-4-(4-methyl-1H-imidazol-1-yl)benzamide;
N-(1-cyclopropyl-2-(5-isopropyl-4-methoxy-2-methylphenyl)ethyl)-3-methoxy-4-(4-methyl-1H-imidazol-1-yl)benzamide;
N-(1-cyclopropyl-2-(4-ethoxy-5-isopropyl-2-methylphenylethyl)-3-methoxy-4-(4-methyl-1H-imidazol-1-yl)benzamide;
N-((6-fluoro-2-oxo-1-(2,2,2-trifluoroethyl)-2,3,4,5-tetrahydro-1H-benzo[b]azepin-3-yl)methyl)-3-methoxy-4-(4-methyl-1H-imidazol-1-yl)benzamide;
3-methoxy-4-(4-methyl-1H-imidazol-1-yl)-N-(2-(1-(2,2,2-trifluoroethyl)-1H-benzo[d]imidazol-2-yl)ethyl)benzamide;
3-methoxy-4-(4-methyl-1H-imidazol-1-yl)-N-(2-(1-(2,2,2-trifluoroethyl)-1H-indol-2-yl)ethyl)benzamide;

and the pharmaceutically acceptable salts of each of the foregoing.

In another embodiment the present invention provides methods of treating neurological and psychiatric disorders comprising: administering to a patient in need thereof an amount of a compound of formula I effective in treating such disorders. Neurological and psychiatric disorders include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering to the mammal an effective amount of compound of formula I or pharmaceutically acceptable salt thereof. Accordingly, in one embodiment, the invention provides a method for treating a condition in a mammal, such as a human, selected from the conditions above, comprising administering a compound of formula I to the mammal. The mammal is preferably a mammal in need of such treatment. As examples, the invention provides a method for treating attention deficit/hyperactivity disorder, schizophrenia and Alzheimer's Disease.

In another embodiment the present invention provides methods of treating neurological and psychiatric disorders comprising: administering to a patient in need thereof an amount of a compound of formula I effective in treating such disorders. The compound of formula I is optionally used in combination with another active agent. Such an active agent may be, for example, an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

The invention is also directed to a pharmaceutical composition comprising a compound of formula I, and a pharmaceutically acceptable carrier. The composition may be, for example, a composition for treating a condition selected from the group consisting of neurological and psychiatric disorders, including but not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering an effective amount of compound of formula I or pharmaceutically acceptable salt thereof, and a pharmaceutically acceptable carrier. The composition optionally further comprises an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

### Definitions

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like. In some instances, the number of carbon atoms in a hydrocarbyl substituent (i.e., alkyl, alkenyl, cycloalkyl, aryl, etc.) is indicated by the prefix “C_{x-y},” wherein x is the minimum and y is the maximum number of carbon atoms in the substituent. Thus, for example, “C₁₋₆alkyl” refers to an alkyl substituent containing from 1 to 6 carbon atoms.

“Alkenyl” refers to an aliphatic hydrocarbon having at least one carbon-carbon double bond, including straight chain, branched chain or cyclic groups having at least one carbon-carbon double bond. Preferably, it is a medium size alkenyl having 2 to 6 carbon atoms. For example, as used herein, the term “C₂₋₆alkenyl” means straight or branched chain unsaturated radicals of 2 to 6 carbon atoms, including, but not limited to ethenyl, 1-propenyl, 2-propenyl (allyl), iso-propenyl, 2-methyl-1-propenyl, 1-butenyl, 2-butenyl, and the like; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or C₁₋₆alkyl. When the compounds of the invention contain a C₂₋₆alkenyl group, the compound may exist as the pure E (entgegen) form, the pure Z (zusammen) form, or any mixture thereof.

“Alkynyl” refers to an aliphatic hydrocarbon having at least one carbon-carbon triple bond, including straight chain, branched chain or cyclic groups having at least one carbon-carbon triple bond. Preferably, it is a lower alkynyl having 2 to 6 carbon atoms. For example, as used herein, the term “C₂₋₆alkynyl” is used herein to mean straight or branched hydrocarbon chain alkynyl radical as defined above having 2 to 6 carbon atoms and one triple bond.

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or to a 5-10-membered heteroaromatic ring may be optionally substituted with halogen, C₁₋₆ alkyl, C₃₋₁₀ cycloalkyl, or ═O.

A cycloalkyl may be a single ring, which typically contains from 3 to 6 ring atoms. Examples include cyclopropyl, cyclobutyl, cyclopentyl, and cyclohexyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄₋₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one or more substituents, the one or more substituents, unless otherwise specified, are each bound to an aromatic carbon of the fused aryl group. The fused C₄₋₁₀ carbocyclic or 4-10-membered heterocyclic ring may be optionally substituted with halogen, C₁₋₆ alkyl, C₃₋₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of atoms in a cyclic substituent containing one or more heteroatoms (i.e., heteroaryl or heterocycloalkyl) is indicated by the prefix “x-y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “cyano” (also referred to as “nitrile”) means —CN, which also may be depicted:

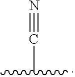

The term “carbonyl” means —C(O)—, which also may be depicted as:

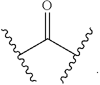

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃), which may also be depicted:

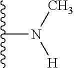

and dialkylamino such as dimethylamino (exemplified by the formula —N(CH₃)₂, which may also be depicted:

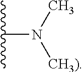

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is fluorine. In another embodiment, the halogen is bromine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The term “oxo” refers to ═O.

The term “oxy” refers to an ether substituent, and may be depicted as —O—.

The term “alkoxy” refers to an alkyl linked to an oxygen, which may also be represented as —OR, wherein the R represents the alkyl group. Examples of alkoxy include methoxy, ethoxy, propoxy and butoxy.

The term “heterocycloalkyl” refers to a substituent obtained by removing a hydrogen from a saturated or partially saturated ring structure containing a total of 4 to 14 ring atoms. At least one of the ring atoms is a heteroatom usually selected from oxygen, nitrogen, or sulfur. A heterocycloalkyl alternatively may comprise 2 or 3 rings fused together, wherein at least one such ring contains a heteroatom as a ring atom (i.e., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆₋₁₀ aromatic ring or to a 5-10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one more substituents, the one or more substituents, unless otherwise specified, are each bound to a heteroatom of the heterocycloalkyl group or to a carbon atom of the heterocycloalkyl group. The fused C₆₋₁₀ aromatic ring or 5-10-membered heteroaromatic ring may be optionally substituted with halogen, C₁₋₆ alkyl, C₃₋₁₀ cycloalkyl, C₁₋₆ alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls and heterocycloalkyls include furanyl, dihydrofuranyl, tetradydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, isoxazolinyl, thiazolyl, isothiazolyl, thiazolinyl, isothiazolinyl, thiazolidinyl, isothiazolidinyl, thiaediazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄₋₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substituents, unless otherwise specified, are each bound to an aromatic carbon of the heteroaryl group or to a heteroatom of the heteroaryl group. The fused C₄₋₁₀ carbocyclic or 4-10-membered heterocyclic ring may be optionally substituted with halogen, C₁₋₆ alkyl, C₃₋₁₀ cycloalkyl, or ═O.

Additional examples of heteroaryls and heterocycloalkyls include: 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-alpha]pyridine, benzothianyl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached).

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated or aromatic. In one embodiment, the heterocyclic ring consists of 4 to 7 atoms. In another embodiment, the heterocyclic ring is selected from the group consisting of pyrrolyl, imidazolyl, pyrazolyl, triazolyl, and tetrazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁₋₆- prefix on C₁₋₆alkylcycloalkyl means that the alkyl moiety of the alkylcycloalkyl contains from 1 to 6 carbon atoms; the C₁₋₆- prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution only occurs on the alkyl moiety, the substituent would be described as “alkoxyhaloalkyl.” If the halogen substitution occurs on both the alkyl moiety and the alkoxy moiety, the substituent would be described as “haloalkoxyhaloalkyl.”

If substituents are described as being “independently selected” from a group, each substituent is selected independent of the other. Each substituent therefore may be identical to or different from the other substituent(s).

As used herein the term “Formula I” are hereinafter referred to as a “compound(s) of the invention.” Such terms are also defined to include all forms of the compound of Formula I, including hydrates, solvates, isomers, crystalline and non-crystalline forms, isomorphs, polymorphs, and metabolites thereof.

### Isomers

When an asymmetric center is present in a compound of formula I, hereinafter referred to as the compound of the invention, the compound may exist in the form of optical isomers (enantiomers). In one embodiment, the present invention comprises enantiomers and mixtures, including racemic mixtures of the compounds of formula I. In another embodiment, for compounds of formula I that contain more than one asymmetric center, the present invention comprises diastereomeric forms (individual diastereomers and mixtures thereof) of compounds. When a compound of formula I contains an alkenyl group or moiety, geometric isomers may arise.

### Tautomeric Forms

The present invention comprises the tautomeric forms of compounds of formula I. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

### Isotopes

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as 2H, ³H, ¹³C, ¹¹C, ¹⁴C, ¹⁵N, ¹⁸O, ¹⁷O, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

### Administration and Dosing

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment of the conditions recited herein.

### Pharmaceutical Compositions

For the treatment of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention.

### General Synthetic Schemes

The compounds of the formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-XII (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

### Experimental Procedures and Working Examples

The following illustrate the synthesis of various compounds of the present invention. Additional compounds within the scope of this invention may be prepared using the methods illustrated in these Examples, either alone or in combination with techniques generally known in the art.

### General Schemes

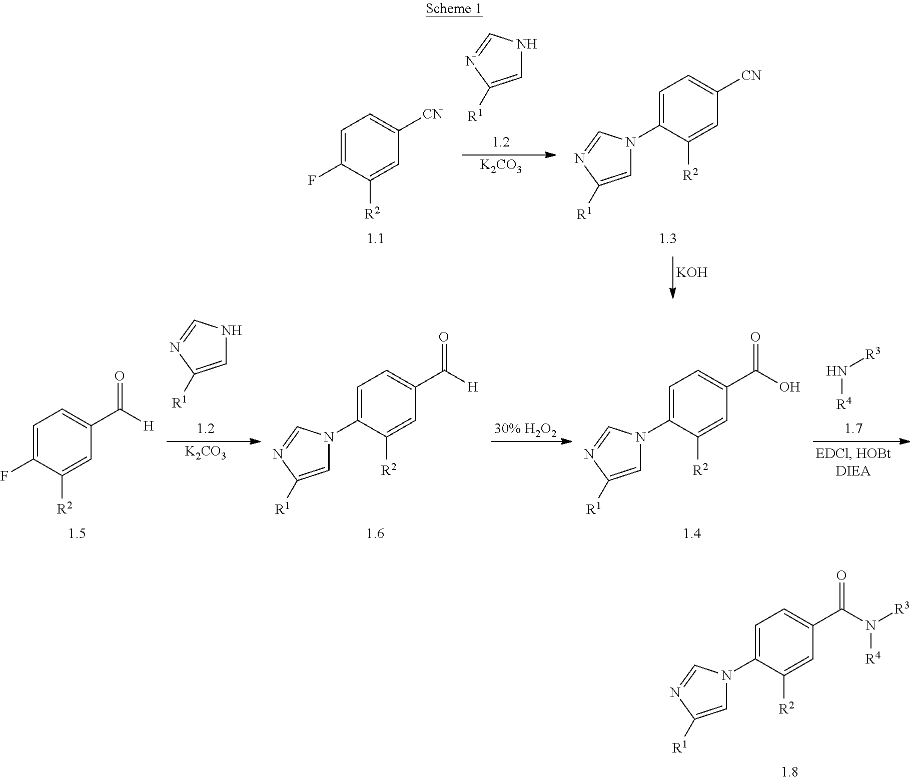

Scheme 1 illustrates a method for preparing compounds depicted by formula 1.8. This method involves the addition of a substituted imidazole of formula 1.2 to an aryl fluoride of formula 1.1 by heating in the presence of a base such as K₂CO₃ or Cs₂CO₃ in a solvent such as DMF, DMAC or DMSO. The corresponding nitrile of formula 1.3 is then hydrolyzed by treating with aqueous KOH to afford a carboxylic acid derivative of formula 1.4. Alternatively, the carboxylic acid of formula 1.4 can be prepared starting from a 4-fluorobenzaldehyde derivative of formula 1.5 using a procedure similar to that described for the addition of imidazole 1.2 to aryl fluoride 1.1. The corresponding substituted benzaldehyde of formula 1.6 is then oxidized to the carboxylic acid of formula 1.4 using 30% H₂O₂ in water. The carboxylic acid of the formula 1.4 can then be coupled to amines of the formula 1.7 using EDCI and HOBT or another suitable coupling reagent in the presence of a base such as diisopropylethylamine to form the corresponding amide of the formula 1.8.

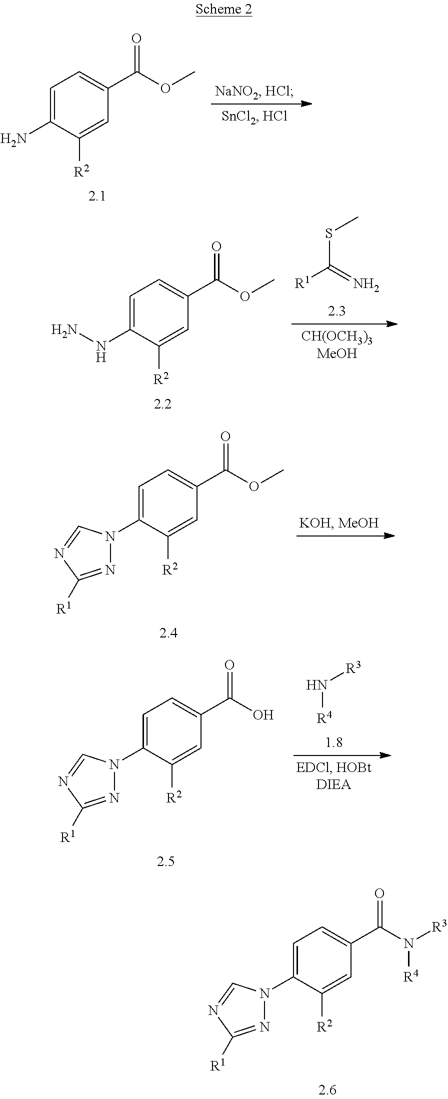

Scheme 2 illustrates a method for preparing phenyl triazole derivatives depicted by formula 2.6. This method commences with the reaction of aniline derivative of formula 2.1 with NaNO₂ in HCl followed by a reduction with a suitable reducing agent such as SnCl₂ to afford the corresponding hydrazine of formula 2.2. The hydrazine of formula 2.2 is then reacted with thioacetimic acid methyl ester (2.3) followed by heating in the presence of HC(OMe)₃ and pyridine to afford the triazole derivative of formula 2.4. The ester function of the compound of formula 2.4 is then hydrolyzed to provide the corresponding carboxylic acid derivative of formula 2.5 by treating with aqueous base such as KOH or LiOH in a solvent such as MeOH or THF. The resulting acid of formula 2.5 is then subjected to amide bond coupling with an amine of the formula 1.8 using EDCI and HOBT or another suitable coupling reagent in the presence of a base such as diisopropylethylamine to form the corresponding amide of the formula 2.6.

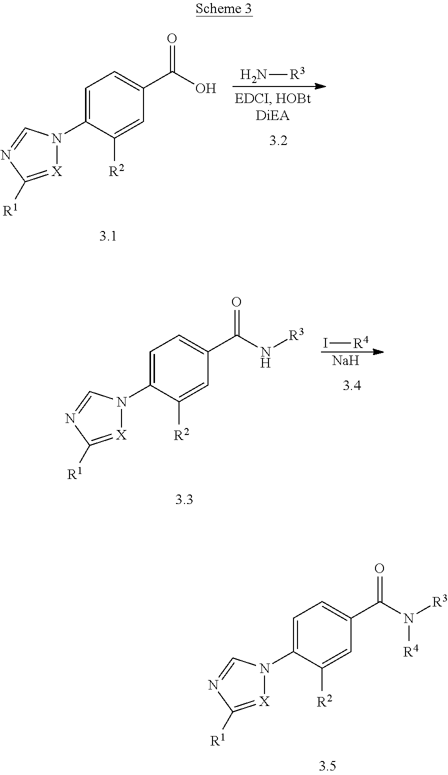

Scheme 3 illustrates a method for preparing amide derivatives depicted by formula 3.5. This method involves the coupling of a carboxylic acid of the formula 3.1 with an amine of the formula 3.2 using EDCI and HOBT or another suitable coupling reagent in the presence of a base such as diisopropylethylamine to form the corresponding amide of the formula 3.3. Compounds of the formula 3.3 can then be alkylated with an alkyl iodide of formula 3.4 or another suitable alkylating agent such as an alkyl bromide or an alkyl triflate. This reaction is carried out in the presence of a base such as NaH, K₂CO₃ or Cs₂CO₃ in a solvent such as DMF.

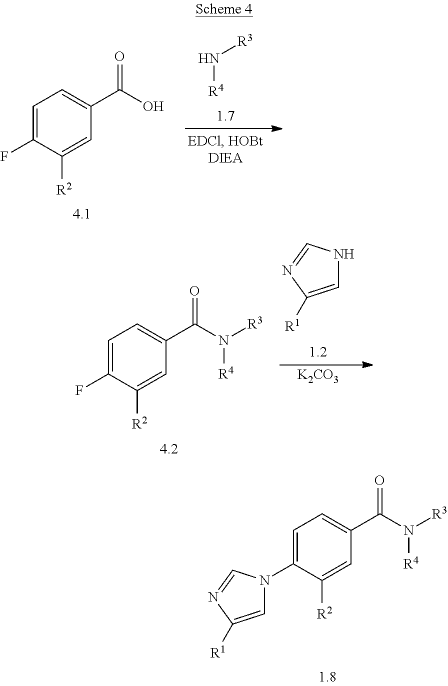

Scheme 4 illustrates a method for preparing amide derivatives depicted by formula 1.8. This method involves the coupling of a carboxylic acid of the formula 4.1 with an amine of the formula 1.7 using EDCI and HOBT or another suitable coupling reagent in the presence of a base such as diisopropylethylamine to form the corresponding amide of the formula 4.2. Compounds of the formula 4.2 can then be subjected to nucleophilic aromatic substitution with heterocycles such as imidazole 1.2 by heating in the presence of a base such as K₂CO₃ or Cs₂CO₃ in a solvent such as DMF, DMAC or DMSO.

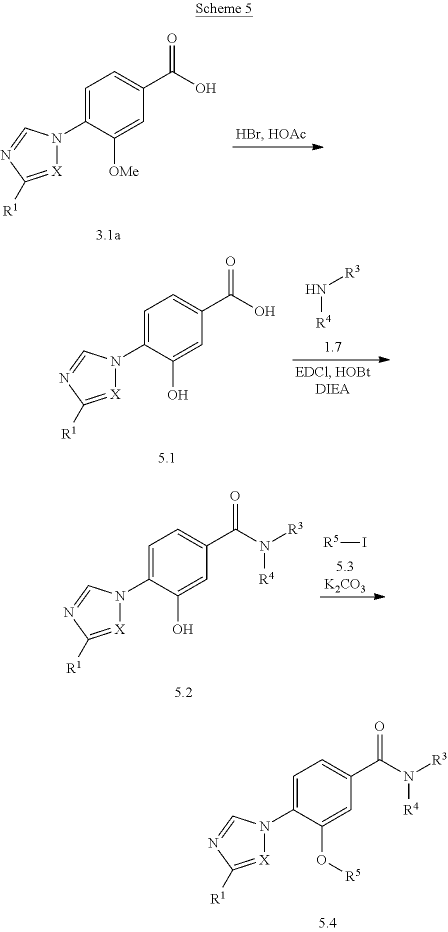

Scheme 5 illustrates a method for preparing compounds of the formula 5.4. This method involves demethylation of a compound of formula 3.1a by heating a mixture of compound 3.1a in concentrated HBr and glacial acetic acid. Alternatively, demethylation of a compound of formula 3.1a can be carried out using BBr₃ in CH₂Cl₂. The corresponding carboxylic acid of the formula 5.1 is then coupled with amines of the formula 1.7 using EDCI and HOBT or another suitable coupling reagent in the presence of a base such as diisopropylethylamine to form the corresponding amide of the formula 5.2. The phenol function in compounds of the formula 5.2 can then be alkylated with alkyl halides of formula 5.3 by stirring in the presence of a base such as K₂CO₃, Cs₂CO₃ or NaH in a solvent such as DMF or CH₂Cl₂. Alternatively, compounds of the formula 5.2 are reacted with alcohols (R⁵OH) under Mitsunobu conditions to provide the corresponding compound of formula 5.4. This reaction is conducted in the presence of an azodicarboxylate such as dibenzyl azodicarboxylate, diethyl azodicarboxylate or di-tert-butyl azodicarboxylate and triphenylphosphine.

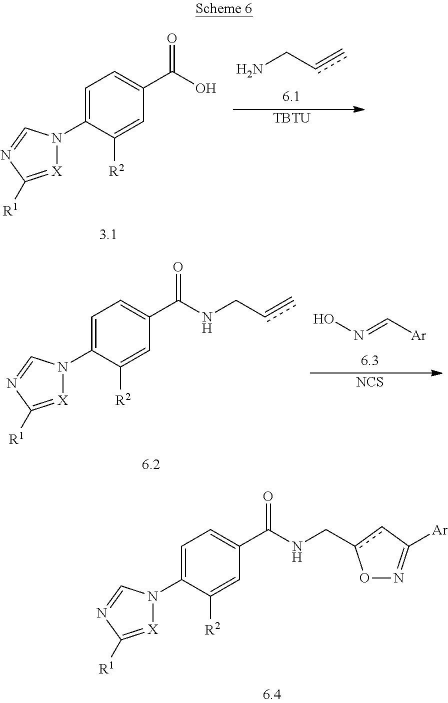

Scheme 6 illustrates a method for preparing amide derivatives depicted by formula 6.4. This method involves the coupling of a carboxylic acid of the formula 3.1 with amines of the formula 6.1 using TBTU or HATU or any other suitable coupling reagent to form the corresponding amide of the formula 6.2. The alkene or alkyne function within a compound of formula 6.2 can then undergo a 3+2 dipolar cycloaddition with an oxime of formula 6.3. This reaction is conducted in the presence of N-chlorosuccinamide or sodium hypochlorite and a base such as triethylamine.

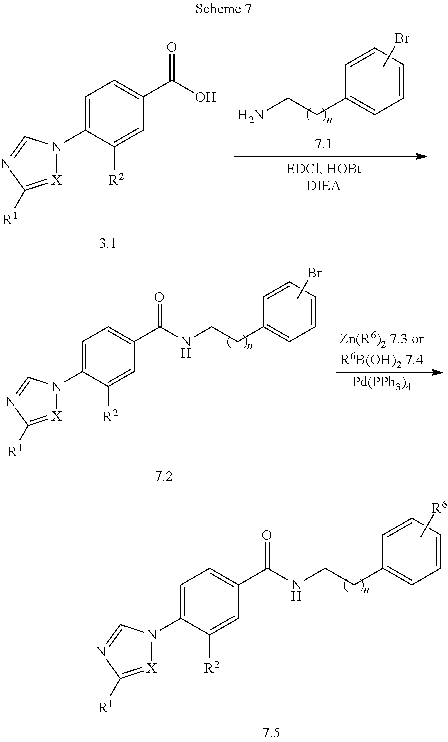

Scheme 7 illustrates a method for preparing amide derivatives depicted by Formula 7.3 (n=0-2) employing methods well known to one skilled in the art. This method involves the coupling of a carboxylic acid of the formula 3.1 with amines of the formula 7.1 (n=0-2) using EDCI and HOBT or another suitable coupling reagent in the presence of a base such as diisopropylethylamine to form the corresponding amide of the formula 7.2. Compounds of the formula 7.2 can be reacted with an organozinc reagent of formula 7.3 under palladium-catalyzed Negishi cross-coupling conditions [Acc. Chem. Res. 1982, 15, 340] or a palladium-catalyzed reaction with Zn(CN)₂ to give the corresponding compounds of formula 7.5. Alternatively, compounds of the formula 7.2 can be reacted with a boronic acid of the formula 7.4 under palladium-catalyzed Suzuki cross-coupling conditions [Chem. Rev. 1995, 95, 2457], to give the corresponding compounds of formula 7.5. For example, the coupling can be conducted using a catalytic amount of tetrakis(triphenylphosphine)-palladium(0) in the presence of a base such as aqueous sodium carbonate, sodium hydroxide, or sodium ethoxide, in a solvent such as THF, dioxane, ethylene glycol dimethyl ether, ethanol (EtOH) or benzene.

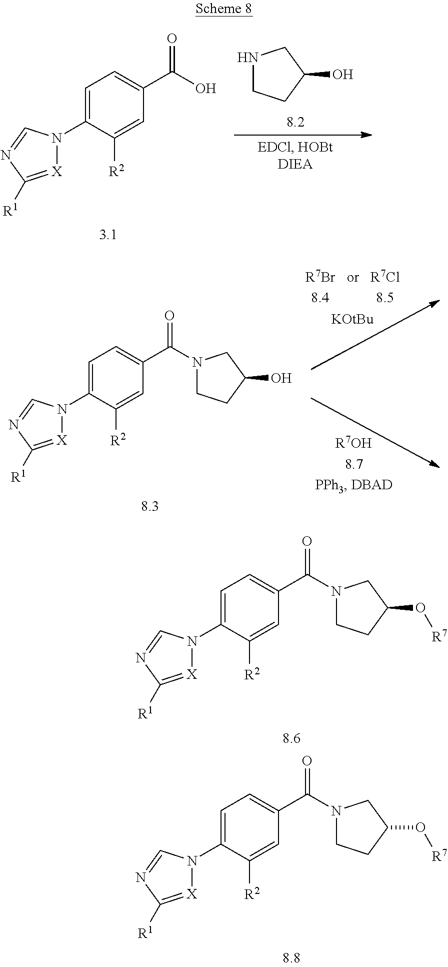

Scheme 8 illustrates a method for preparing compounds of the formula 8.6 and 8.8. Referring to Scheme 5, compounds of the formula 3.1 can be reacted with amines such as (S)-pyrrolidin-3-ol (8.2) using EDCI and HOBT or another suitable coupling reagent in the presence of a base such as diisopropylethylamine to form the corresponding amide of the formula 8.3. Addition of a compound of the formula 8.3 to an alkyl halide of the formula 8.4 or 8.5 provides the corresponding compound of formula 8.6. This reaction is generally conducted in the presence of a base such as potassium tert-butoxide, sodium tert-butoxide, sodium hydride, potassium hydride, lithium diisopropylamide, lithium bis(trimethylsilyl)amide, potassium bis(trimethylsilyl)amide, or sodium bis(trimethylsilyl)amide. Suitable solvents for this reaction include THF, dioxane, ethylene glycol dimethyl ether, DMF, NMP, and DMSO, or a combination of two or more of these solvents. Alternatively, compounds of the formula 8.3 are reacted with alcohols of the formula 8.7 (R⁷OH) under Mitsunobu conditions to provide the corresponding compound of formula 8.8. This reaction is conducted in the presence of an azodicarboxylate such as dibenzyl azodicarboxylate, diethyl azodicarboxylate or di-tert-butyl azodicarboxylate and triphenylphosphine.

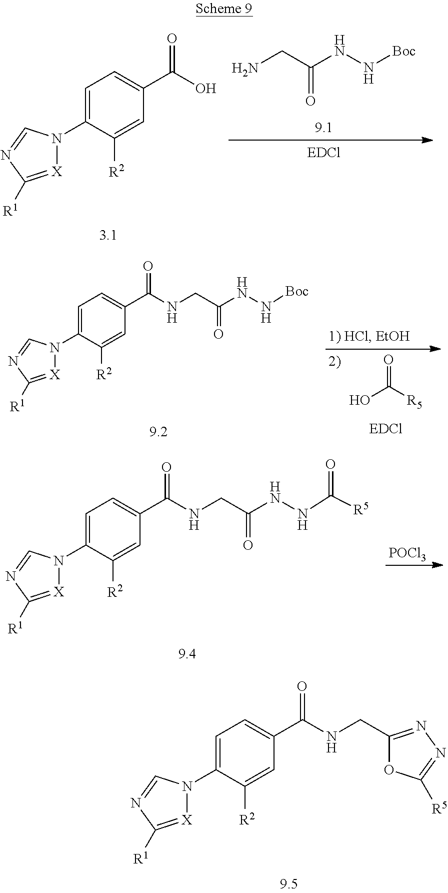

Scheme 9 illustrates a method for preparing compounds of the formula 9.5. A compound of the formula 3.1 can be reacted with N′-(2-aminoacetyl)hydrazinecarboxylic acid tert-butyl ester (9.1) using EDCI or another suitable coupling reagent in a solvent such as CH₂Cl₂ to form the corresponding amide of the formula 9.2. Removal of the Boc-protecting group of a compound of formula 9.2 using an acid such as HCl or TFA is followed by a peptide coupling reaction with a carboxylic acid derivative of formula 9.3 using EDCI, to afford the corresponding amide of the formula 9.4. Compounds of the formula 9.4 can then undergo cyclodehydration upon exposure to a suitable dehydrating agent such as POCl₃ to provide compound 9.5.

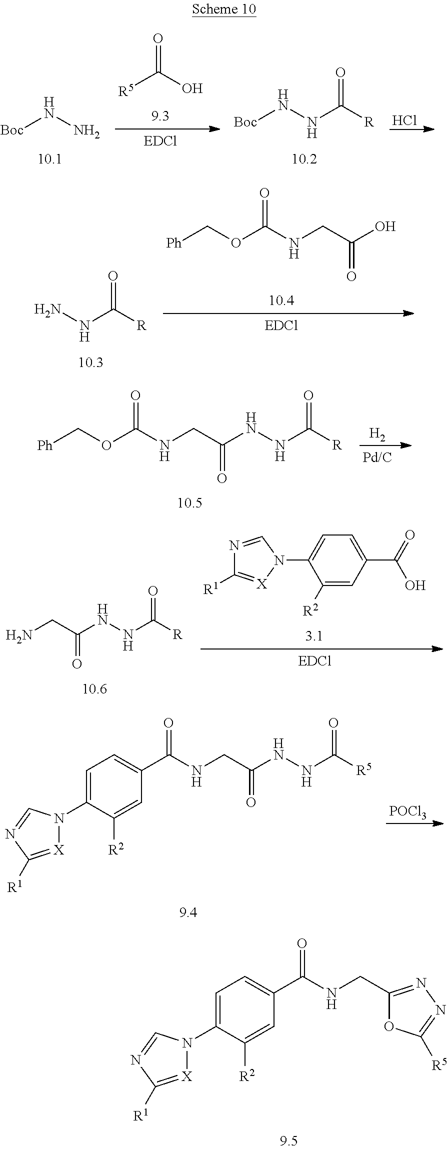

Scheme 10 illustrates an alternative method for preparing compounds of the formula 9.5. A compound of the formula 10.1 can be reacted with a carboxylic acid derivative of formula 9.3 using EDCI or another suitable coupling reagent in a solvent such as CH₂Cl₂ to form the corresponding amide of the formula 10.2. Removal of the Boc-protecting group of the compound of formula 10.2 using an acid such as HCl or TFA is followed by peptide coupling reaction with an acid of formula 10.4 to afford the corresponding amide of the formula 10.5. The compound of formula 10.5 is subjected to hydrogenolysis to remove the CBz protecting group to provide a compound of formula 10.6, which in turn is coupled with carboxylic acid derivative 3.1 using EDCI to afford a compound of formula 9.4. As in scheme 9, compounds of the formula 9.4 can then undergo a cyclodehydration upon exposure to a suitable dehydrating agent such as POCl₃ to provide compound 9.5.

It will be understood that the intermediate compounds of the invention depicted above are not limited to the particular enantiomer shown, but also include all stereoisomers and mixtures thereof. It will also be understood that compounds of Formula I can be used as intermediates for compounds of Formula I.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS) or atmospheric pressure chemical ionization (APCI) instrumentation. Chemical shifts for nuclear magnetic resonance (NMR) data are expressed in parts per million (ppm, δ) referenced to residual peaks from the deuterated solvents employed.

### EXAMPLE 1

### Synthesis of N-[2-(3-cyanophenyl)ethyl]-3-methoxy-4-(4-methyl-1H-imidazol-1-yl)benzamide (1)

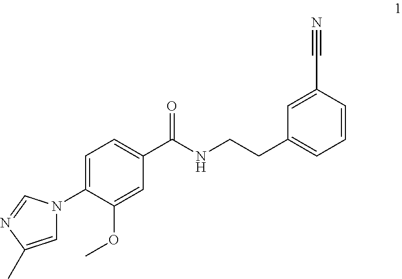

Step 1. Synthesis of 3-methoxy-4-(4-methyl-1H-imidazol-1-yl)benzoic Acid.

A. Synthesis of 3-methoxy-4-(4-methyl-1H-imidazol-1-yl)benzonitrile. 4-Fluoro-3-methoxybenzonitrile (98.2 g, 0.65 mol) was combined with 4-methyl-1H-imidazole (53.4 g, 0.65 mol) and anhydrous potassium carbonate (138.2 g, 1 mol, 1.54 eq) in dimethylformamide (650 mL), and the reaction mixture was stirred for 16 hours at 135-140° C. (internal temperature). The mixture was cooled to room temperature and filtered; the salts were washed with dichloromethane (3×30 mL). The combined filtrates were evaporated in vacuo to afford a brown semisolid, which was suspended in water (500 mL) and left to stand at 5° C. for 24 hours. The mixture was filtered, and the solid was washed with ice water (2×50 mL), then dried in vacuo to afford a yellow solid (105.5 g). Crystallization from methanol (106 mL) provided purified product (66.3 g) as yellowish crystals. These were boiled with acetone (100 mL) for 5 minutes and the mixture was left to cool overnight. The mixture was filtered, and the crystals were washed with acetone (2×10 mL) to afford the title compound as a white solid. Yield: 54.6 g, 0.256 mmol, 39%. ¹H NMR (400 MHz, CDCl₃) δ 2.31 (d, J=1.0 Hz, 3H), 3.94 (s, 3H), 6.97 (m, 1H), 7.30 (m, 1H), 7.37 (m, 2H), 7.79 (d, J=1.4 Hz, 1H).

B. Synthesis of 3-methoxy-4-(4-methyl-1H-imidazol-1-yl)benzoic acid. 3-Methoxy-4-(4-methyl-1H-imidazol-1-yl)benzonitrile (46.9 g, 0.22 mol), in methanol (176 mL) and water (88 mL), were treated with potassium hydroxide (85%, 16.5 g, 0.25 mol) and the resulting solution was refluxed for 72 hours. The solution was concentrated in vacuo to approximately 100 mL, and the resulting solid was removed by filtration. Water (40 mL) was added to the filtrate, which was then acidified to pH 5 by addition of small portions of 30% aqueous hydrochloric acid (approximately 40 mL). The resulting thick suspension was heated to reflux for 5 minutes and left to cool overnight. The mixture was filtered, and the white solid was washed with water (3×20 mL) and dried in vacuo to afford the title compound as a white powder. Yield: 53.76 g, 0.231 mmol, quantitative. LCMS m/z 233.1 (M+1). ¹H NMR (300 MHz, CD₃OD) δ 2.36 (d, J=1.0 Hz, 3H), 3.97 (s, 3H), 7.43 (m, 1H), 7.56 (d, J=8.2 Hz, 1H), 7.76 (dd, J=8.1, 1.7 Hz, 1H), 7.84 (d, J=1.6 Hz, 1H), 8.66 (d, J=1.5 Hz, 1H).

Step 2. Synthesis of N-[2-(3-bromophenyl)ethyl]-3-methoxy-4-(4-methyl-1H-imidazol-1-yl)benzamide. 2-(3-Bromophenyl)ethanamine (427 mg, 2.14 mmol) was combined with 3-methoxy-4-(4-methyl-1H-imidazol-1-yl)benzoic acid (517 mg, 2.14 mmol) and O-(1H-benzotriazol-1-yl)-1,1,3,3-tetramethyluronium hexafluorophosphate (HBTU, 1.00 g, 2.56 mmol) in dimethylformamide (4.27 mL) and diisopropylethylamine (0.76 mL, 4.3 mmol), and the resulting suspension was stirred at room temperature for 18 hours. The reaction mixture was partitioned between ethyl acetate and water, and the aqueous layer was extracted with ethyl acetate (2×20 mL). The combined organic layers were dried over sodium sulfate, filtered and concentrated in vacuo. The residue was purified twice via silica gel chromatography

(Gradient: 0% to 5% [2M ammonia in methanol] in ethyl acetate), then subjected to

HPLC purification (Column: Phenomenex Gemini C₁₈; Mobile phase A: 0.1% ammonium hydroxide in water; Mobile phase B: 0.1% ammonium hydroxide in acetonitrile; Gradient: 50% B to 80% B) to provide the title compound as an oil. Yield: 360 mg, 0.869 mmol, 41%. LCMS m/z 416.1 (M+1). ¹H NMR (400 MHz, CD₃OD) 2.24 (d, J=1.0 Hz, 3H), 2.92 (t, J=7.1 Hz, 2H), 3.61 (t, J=7.2 Hz, 2H), 3.92 (s, 3H), 7.11 (m, 1H), 7.18-7.25 (m, 2H), 7.36 (ddd, J=7.4, 1.9, 1.9 Hz, 1H), 7.40-7.47 (m, 3H), 7.57 (d, J=1.8 Hz, 1H), 7.85 (d, J=1.4 Hz, 1H).

Step 3. Synthesis of N-[2-(3-cyanophenyl)ethyl]-3-methoxy-4-(4-methyl-1H-imidazol-1-yl)benzamide (1). Zinc cyanide (85%, 106 mg, 0.77 mmol) and compound N-[2-(3-bromophenyl)ethyl]-3-methoxy-4-(4-methyl-1H-imidazol-1-yl)benzamide (310 mg, 0.748 mmol) were mixed with dimethylformamide (10 mL) and degassed for 5 minutes. Tetrakis(triphenylphosphine)palladium (43 mg, 0.037 mmol) was added, and the solution was degassed for an additional 10 minutes, then stirred at 100° C. for 18 hours. The reaction mixture was cooled to room temperature and treated with water (20 mL), 1N aqueous sodium hydroxide solution (10 mL) and ethyl acetate (10 mL). The aqueous layer was extracted with ethyl acetate (2×10 mL), and the combined organic layers were dried over sodium sulfate, filtered and concentrated in vacuo. Purification via silica gel chromatography (Gradient: 0% to 5% [2M ammonia in methanol] in ethyl acetate) provided title compound as a solid. Yield: 170 mg, 0.472 mmol, 63%. LCMS m/z 361.5 (M+1). ¹H NMR (500 MHz, CDCl₃) δ 2.25 (s, 3H), 3.00 (t, J=7.1 Hz, 2H), 3.71 (br dt, J=6.7, 6.7 Hz, 2H), 3.87 (s, 3H), 6.93 (s, 1H), 7.07 (br t, J=6 Hz, 1H), 7.24 (d, J=8.1 Hz, 1H), 7.31 (dd, J=8.1, 1.7 Hz, 1H), 7.41 (dd, J=8, 8 Hz, 1H), 7.48-7.52 (m, 3H), 7.57 (d, J=1.7 Hz, 1H), 7.65 (br s, 1H).

### EXAMPLE 2

### Synthesis of N-[2-(4-chlorophenyl)-2-hydroxypropyl]-3-methoxy-4-(4-methyl-1H-imidazol-1-yl)benzamide (2)

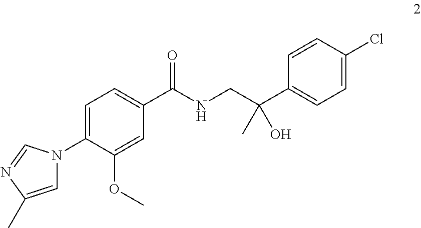

Step 1. Synthesis of N-[2-(4-chlorophenyl)-2-oxoethyl]-3-methoxy-4-(4-methyl-1H-imidazol-1-yl)benzamide. 3-Methoxy-4-(4-methyl-1H-imidazol-1-yl)benzoic acid [Example 1], (500 mg, 2.15 mmol), 2-amino-1-(4-chlorophenyl)ethanone (444 mg, 2.15 mmol), O-(7-azabenzotriazol-1-yl)-1,1,3,3-tetramethyluronium hexafluorophosphate (HATU, 0.911 g, 2.32 mmol) and diisopropylethylamine (1.5 mL, 0.86 mmol) were combined in dimethylformamide (10 mL). The mixture was stirred at room temperature for 16 hours, then diluted with saturated aqueous sodium bicarbonate solution (200 mL) and extracted with ethyl acetate (2×200 mL). The organic layers were combined and washed with saturated aqueous sodium bicarbonate solution (2×200 mL), water (2×200 mL) and saturated aqueous sodium chloride solution (150 mL). The organic layer was dried over magnesium sulfate, concentrated in vacuo, and purified via silica gel chromatography (Mobile phase A: ethyl acetate; Mobile phase B: 1:9 [2N ammonia in methanol]: ethyl acetate; Gradient: 0%-70% B) to provide the title compound as a white solid. Yield: 546 mg, 1.42 mmol, 66%. LCMS m/z 384.1 (M+1). ¹H NMR (400 MHz, CDCl₃) δ 2.31 (d, J=1.0 Hz, 3H), 3.94 (s, 3H), 4.94 (d, J=4.3 Hz, 2H), 6.98 (m, 1H), 7.33-7.36 (m, 2H), 7.48 (dd, J=8.1, 1.8 Hz, 1H), 7.52 (br d, J=8.9 Hz, 2H), 7.63 (d, J=1.8 Hz, 1H), 7.79 (d, J=1.4 Hz, 1H), 7.99 (br d, J=8.8 Hz, 2H).

Step 2. Synthesis of N-[2-(4-chlorophenyl)-2-hydroxypropyl]-3-methoxy-4-(4-methyl-1H-imidazol-1-yl)benzamide (2). A 3 M solution of methylmagnesium bromide in tetrahydrofuran (1.32 mL, 3.96 mmol) was added to a solution of N-[2-(4-chlorophenyl)-2-oxoethyl]-3-methoxy-4-(4-methyl-1H-imidazol-1-yl)benzamide (152 mg, 0.396 mmol) in tetrahydrofuran (5 mL) over 10 minutes. The reaction was stirred at room temperature for 2 hours, whereupon saturated aqueous sodium bicarbonate solution (10 mL) was added. The mixture was extracted with ethyl acetate (3×50 mL), and the combined organic layers were dried over magnesium sulfate, and concentrated under reduced pressure. Purification via silica gel chromatography (Mobile phase A: ethyl acetate; Mobile phase B: 1:9 [2N ammonia in methanol]: ethyl acetate; Gradient: 0%-70% B) afforded the title compound as a clear gum. Yield: 15 mg, 0.038 mmol, 10%. LCMS m/z 400.3 (M+1). ¹H NMR (400 MHz, CDCl₃) 1.61 (s, 3H), 2.26 (s, 3H), 3.73 (dd, J=14.0, 5.8 Hz, 1H), 3.85 (s, 3H), 3.87 (m, 1H), 6.88-6.91 (m, 2H), 7.11-7.16 (m, 2H), 7.32 (br d, J=8.6 Hz, 2H), 7.44-7.48 (m, 3H), 7.54 (br s, 1H).

### EXAMPLE 3

### Synthesis of N-[2-(3-chlorophenyl)ethyl]-4-(4-methyl-1H-imidazol-1-yl)-3-(prop-2-yn-1-yloxy)benzamide (3)

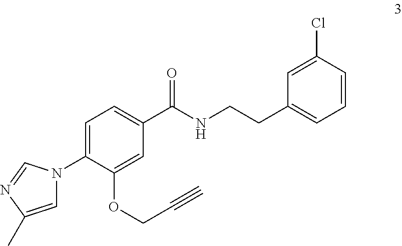

Step 1. Synthesis of 3-methoxy-4-(4-methyl-1H-imidazol-1-yl)benzaldehyde. 4-Fluoro-3-methoxybenzaldehyde (85 g, 0.55 mol), 4-methyl-1H-imidazole (90.5 g, 1.1 mol) and cesium carbonate (268.8 g, 0.82 mol) were combined in dimethylformamide (1.7 L) and stirred at 100° C. for 1 hour. The mixture was cooled to room temperature, filtered, and the filtrate was concentrated in vacuo. The residue was dissolved in water (2 L) and extracted with ethyl acetate (3×2 L). The combined organic layers were washed with water and brine, dried over sodium sulfate, and concentrated under reduced pressure. Chromatography on silica (Eluant: 2:1 petroleum ether: ethyl acetate) afforded a yellow solid, which was recrystallized from ethyl acetate (300 mL) to provide the title compound as a white solid. Yield: 17.8 g, 0.082 mol, 15%. ¹H NMR (300 MHz, CDCl₃) δ 2.31 (d, J=1.0 Hz, 3H), 3.97 (s, 3H), 7.01 (m, 1H), 7.45 (d, J=7.8 Hz, 1H), 7.54-7.58 (m, 2H), 7.83 (d, J=1.3 Hz, 1H), 10.01 (s, 1H).

Step 2. Synthesis of 3-methoxy-4-(4-methyl-1H-imidazol-1-yl)benzoic acid, Hydrochloride Salt.

Aqueous hydrogen peroxide (30%, 18.9 mL) was added drop-wise over 20 minutes to a solution of 3-methoxy-4-(4-methyl-1H-imidazol-1-yl)benzaldehyde (5.0 g, 20.0 mmol) and potassium hydroxide (6.1 g, 92.5 mmol) in MeOH (38 mL) and water (6.6 mL) at 65° C. Following completion of the addition the reaction was stirred at room temperature for an additional 25 minutes. The reaction was then allowed to cool to room temperature and was acidified with conc. HCl. The resulting precipitate was filtered to provide the title compound as a white solid. Yield: 4.6 g, 17.1 mmol, 74%. MS (APCI) m/z 232.9 (M+1). ¹H NMR (400 MHz, CD₃OD) 2.45 (br s, 3H), 4.00 (s, 3H), 7.65 (br s, 1H), 7.67 (d, J=8.4 Hz, 1H), 7.82 (dd, J=8.3, 1.6 Hz, 1H), 7.89 (d, J=1.6 Hz, 1H), 9.24 (d, J=1.6 Hz, 1H).

Step 3. Synthesis of 3-hydroxy-4-(4-methyl-1H-imidazol-1-yl)benzoic acid, hydrobromide salt. Aqueous hydrobromic acid (48%, 25 mL) and acetic acid (25 mL) were added to a flask charged with 3-methoxy-4-(4-methyl-1H-imidazol-1-yl)benzoic acid, hydrochloride salt (2.0 g, 7.4 mmol), and the resulting slurry was heated at reflux (bath temperature 150° C.) for 72 hours. The reaction was allowed to cool to room temperature, and then cooled further in an ice bath, resulting in the formation of a precipitate. Ice cold water (10 mL) was added, and the solid was isolated by filtration, washed with additional ice cold water (10 mL) and dried under high vacuum for to afford the title compound as a white solid. Yield: 2.245 g, 7.50 mmol, quantitative. LCMS m/z 219.2 (M+1). ¹H NMR (400 MHz, DMSO-d₆) δ 2.35 (d, J=1.2 Hz, 3H), 7.55 (dd, J=8.2, 1.8 Hz, 1H), 7.64 (d, J=8.3 Hz, 1H), 7.71 (d, J=1.8 Hz, 1H), 7.80 (m, 1H), 9.41 (d, J=1.7 Hz, 1H), 11.21 (s, 1H), 13.3 (br s, 1H).

Step 4. Synthesis of N-[2-(3-chlorophenyl)ethyl]-3-hydroxy-4-(4-methyl-1H-imidazol-1-yl)benzamide. 3-Hydroxy-4-(4-methyl-1H-imidazol-1-yl)benzoic acid, hydrobromide salt from the previous experiment (300 mg, 1.00 mmol) was combined with 2-(3-chlorophenyl)ethanamine (321 mg, 2.06 mmol), 1H-benzotriazol-1-ol (HOBT, 279 mg, 2.06 mmol), diisopropylethylamine (0.94 mL, 5.50 mmol) in dimethylformamide (6.9 mL) and the mixture was stirred until dissolved. N-[3-(dimethylamino)propyl]-N′-ethylcarbodiimide hydrochloride (EDCI, 320 mg, 2.06 mmol) was added, and the reaction was stirred for 18 hours. The reaction mixture was poured into water (100 mL) and extracted with ethyl acetate (3×100 mL). The combined organic layers were dried over magnesium sulfate, filtered and concentrated under reduced pressure. Chromatography on silica (Mobile phase A: ethyl acetate; Mobile phase B: 9:1 ethyl acetate: [2M ammonia in methanol]; Gradient: 50% B to 100% B) afforded the title compound. Yield: 280 mg, 0.787 mmol, 77%. LCMS m/z 356.1, 358.1 (M+1). ¹H NMR (400 MHz, CD₃OD) δ 2.24 (br s, 3H), 2.91 (t, J=7.3 Hz, 2H), 3.58 (t, J=7.3 Hz, 2H), 7.16-7.22 (m, 3H), 7.25-7.30 (m, 3H), 7.37 (d, J=8.3 Hz, 1H), 7.44 (d, J=1.9 Hz, 1H), 7.94 (d, J=1.2 Hz, 1H).

Step 5. Synthesis of N-[2-(3-chlorophenyl)ethyl]-4-(4-methyl-1H-imidazol-1-yl)-3-(prop-2-yn-1-yloxy)benzamide (3). 3-Bromoprop-1-yne (80% in toluene, 57 μL, 0.57 mmol) was added to a mixture of N-[2-(3-chlorophenyl)ethyl]-3-hydroxy-4-(4-methyl-1H-imidazol-1-yl)benzamide (102 mg, 0.287 mmol) and potassium carbonate (99 mg, 0.72 mmol) in dimethylformamide (2.9 mL), and the reaction was stirred for 18 hours at room temperature. The reaction mixture was poured into ethyl acetate (100 mL) and was washed with water (20 mL) and brine (3×20 mL), dried over magnesium sulfate, and concentrated in vacuo. Chromatography on silica (Mobile phase A: ethyl acetate; Mobile phase B: 9:1 ethyl acetate: [2M ammonia in methanol]; Gradient: 0% to 50% B) afforded title compound. Yield: 70 mg, 0.18 mmol, 63%. LCMS m/z 394.2 (M+1). ¹H NMR (400 MHz, CDCl₃) δ 2.29 (d, J=1.0 Hz, 3H), 2.54 (t, J=2.4 Hz, 1H), 2.95 (t, J=6.9 Hz, 2H), 3.73 (dt, J=6.0, 6.9 Hz, 2H), 4.78 (d, J=2.4 Hz, 2H), 6.32 (br t, J=6 Hz, 1H), 6.96 (m, 1H), 7.14 (m, 1H), 7.23-7.28 (m, 3H), 7.29-7.31 (m, 2H), 7.65 (m, 1H), 7.74 (d, J=1.3 Hz, 1H).

### EXAMPLE 4

### Synthesis of N-{[3-(3-chlorophenyl)isoxazol-5-yl]methyl}-3-methoxy-4-(4-methyl-1H-imidazol-1-yl)benzamide (4)

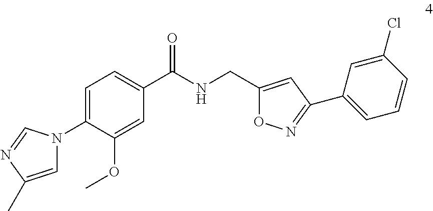

Step 1. Synthesis of 3-chlorobenzaldehyde oxime. Hydroxylamine hydrochloride 593 mg, 8.54 mmol) was added to a solution of 3-chlorobenzaldehyde (0.81 mL, 7.1 mmol) in pyridine (4 mL), and the reaction mixture was stirred for 18 hours at room temperature. The reaction was concentrated under reduced pressure, and the residue was partitioned between 10% aqueous sodium carbonate solution and ethyl acetate. The aqueous layer was extracted with ethyl acetate, and the combined organic layers were concentrated under reduced pressure to afford the title compound (contaminated with pyridine), which was used without purification in Step 3 below. Yield: 1.5 g, assumed quantitative. ¹H NMR (500 MHz, CD₃OD), product peaks only: δ 7.33-7.35 (m, 2H), 7.48 (m, 1H), 7.61 (m, 1H), 8.05 (s, 1H).

Step 2. Synthesis of 3-methoxy-4-(4-methyl-1H-imidazol-1-yl)-N-prop-2-yn-1-ylbenzamide. Pyridine (550 μL, 6.8 mmol) and O-(1H-benzotriazol-1-yl)-1,1,3,3-tetramethyluronium tetrafluoroborate (TBTU, 1.32 g, 4.11 mmol) was added to a mixture of 3-methoxy-4-(4-methyl-1H-imidazol-1-yl)benzoic acid hydrochloride salt [Example 3], (795 mg, 3.00 mmol) and prop-2-yn-1-amine (263 μL, 4.11 mmol) in dimethylformamide (10 mL). The reaction mixture was stirred for 18 hours at room temperature, whereupon it was concentrated using a high vacuum Genevac HT-4 system, to provide crude title compound contaminated with pyridine and 1H-benzotriazol-1-ol. Assumed quantitative. LCMS m/z 270.3 (M+1). ¹H NMR (500 MHz, CD₃OD), product peaks only: 2.44 (d, J=1.1 Hz, 3H), 2.64 (t, J=2.6 Hz, 1H), 4.00 (s, 3H), 4.19 (d, J=2.4 Hz, 2H), 7.61-7.63 (m, 2H), 7.66 (d, J=8.3 Hz, 1H), 7.75 (m, 1H), 9.20 (d, J=1.6 Hz, 1H).

Step 3. Synthesis of N-{[3-(3-chlorophenyl)isoxazol-5-yl]methyl}-3-methoxy-4-(4-methyl-1H-imidazol-1-yl)benzamide (4). N-Chlorosuccinimide (99.6 mg, 0.746 mmol) was added to a solution of 3-chlorobenzaldehyde oxime (116 mg, <0.746 mmol) in dichloromethane (15 mL), and the resulting solution was stirred for 1 hour at room temperature. 3-Methoxy-4-(4-methyl-1H-imidazol-1-yl)-N-prop-2-yn-1-ylbenzamide from the previous step (22% of the product obtained, 0.67 mmol) was added as a solution in dichloromethane (8 mL), followed by drop-wise addition of triethylamine (0.104 mL, 0.746 mmol). The reaction was stirred for 18 hours at room temperature, whereupon it was quenched with aqueous sodium bicarbonate solution. The reaction mixture was extracted with dichloromethane (2×50 mL), and the combined organic layers were dried over sodium sulfate and concentrated under reduced pressure. The residue was dissolved in dimethyl sulfoxide (4 mL) and purified by HPLC (Column: Waters XBridge C₁₈, 5 μm; Mobile phase A: 0.1% trifluoroacetic acid in water; Mobile phase B: 0.1% trifluoroacetic acid in acetonitrile; Gradient: 5% B to 50% B), to afford title compound as a colorless liquid. Yield 15 mg, 0.035 mmol, 5% over 2 steps. LCMS m/z 423.6, 425.6 (M+1). ¹H NMR (500 MHz, CD₃OD) δ 2.25 (d, J=1.0 Hz, 3H), 3.97 (s, 3H), 4.78 (s, 2H), 6.83 (m, 1H), 7.15 (m, 1H), 7.45-7.50 (m, 3H), 7.60 (dd, J=8.2, 1.8 Hz, 1H), 7.72 (d, J=1.7 Hz, 1H), 7.77 (m, 1H), 7.87 (m, 1H), 7.91 (d, J=1.3 Hz, 1H).

### EXAMPLE 5

### Synthesis of N-{[5-(3-chlorophenyl)-1,3,4-oxadiazol-2-yl]methyl}-3-methoxy-4-(4-methyl-1H-imidazol-1-yl)benzamide (5)

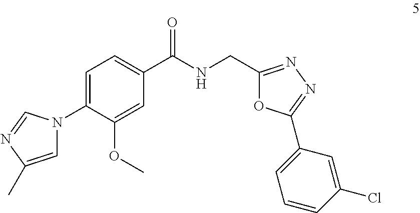

Step 1. Synthesis of tert-butyl 2-(aminoacetyl)hydrazinecarboxylate.

A. Synthesis of tert-butyl 2-({[(benzyloxy)carbonyl]amino}acetyl)hydrazinecarboxylate. N-[3-(Dimethylamino)propyl]-N′-ethylcarbodiimide hydrochloride (EDCI, 4.12 g, 21.5 mmol) was added to a solution of N—[(benzyloxy)carbonyl]glycine (3.00 g, 14.3 mmol) and tert-butyl hydrazinecarboxylate (2.65 g, 20.0 mmol) in dichloromethane (50 mL). The reaction mixture was stirred at room temperature for 6 hours, whereupon it was washed with saturated aqueous sodium bicarbonate solution and with water. The organic layer was dried over magnesium sulfate and concentrated under reduced pressure. Chromatography on silica (Gradient: 50% to 75% ethyl acetate in heptane) provided the title compound as a white gum. Yield: 2.26 g, 7.00 mmol, 49%. LCMS m/z 322.1 (M−1). ¹H NMR (500 MHz, CDCl₃) δ 1.46 (s, 9H), 3.94 (br d, J=5.9 Hz, 2H), 5.13 (s, 2H), 7.35 (m, 5H).

B. Synthesis of tert-butyl 2-(aminoacetyl)hydrazinecarboxylate. 10% Palladium on carbon (300 mg) was added to a solution of tert-butyl 2-({[(benzyloxy)carbonyl]amino}acetyl)-hydrazinecarboxylate (2.26 g, 7.00 mmol) in methanol (25 mL) and the mixture was hydrogenated at 50 psi for 3 hours. The reaction mixture was then filtered through Celite, and washed with methanol, and the combined filtrates were concentrated in vacuo to afford the title compound as a light grey solid. Yield: 1.28 g, 6.76 mmol, 97%. ¹H NMR (500 MHz, CDCl₃) δ 1.49 (s, 9H), 3.50 (br s, 2H).

Step 2. Synthesis of tert-butyl 2-({[3-methoxy-4-(4-methyl-1H-imidazol-1-yl)benzoyl]amino}acetyl)hydrazinecarboxylate. N-[3-(Dimethylamino)propyl]-N′-ethylcarbodiimide hydrochloride (EDCI, 1.69 g, 8.82 mmol) was added to a solution of 3-methoxy-4-(4-methyl-1H-imidazol-1-yl)benzoic acid [Example 1] (1.57 g, 6.76 mmol) and tert-butyl 2-(aminoacetyl)hydrazinecarboxylate (1.28 g, 6.76 mmol) in dichloromethane (30 mL). The reaction was stirred at room temperature for 18 hours, whereupon it was washed with saturated aqueous sodium bicarbonate solution, water, and brine. The organic layer was dried over magnesium sulfate, and concentrated to provide the title compound, which was used in the next step without purification. Yield: 2.26 g, 5.60 mmol, 83%. MS (APCI) m/z 401.8 (M−1). ¹H NMR (500 MHz, CDCl₃) δ 1.47 (s, 9H), 2.29 (br s, 3H), 3.89 (s, 3H), 4.22 (d, J=5.5 Hz, 2H), 6.60 (br s, 1H), 6.92 (s, 1H), 7.24 (d, J=8.1 Hz, 1H), 7.40 (dd, J=8.1, 1.7 Hz, 1H), 7.56 (br s, 1H), 7.74 (br s, 1H).

Step 3. Synthesis of N-(2-hydrazino-2-oxoethyl)-3-methoxy-4-(4-methyl-1H-imidazol-1-yl)benzamide, hydrochloride salt. To a flask charged with tert-butyl 2-({[3-methoxy-4-(4-methyl-1H-imidazol-1-yl)benzoyl]amino}acetyl)hydrazinecarboxylate (2.26 g, 5.60 mmol) was added a 5% solution of hydrogen chloride in ethanol (20 mL). The reaction was stirred at room temperature for 18 hours, whereupon it was concentrated under reduced pressure to afford the title compound as a white solid. Yield: 1.754 g, 5.16 mmol, 92%. MS (APCI) m/z 301.8 (M−1). ¹H NMR (500 MHz, DMSO-d₆) δ 2.36 (d, J=1.1 Hz, 3H), 3.95 (s, 3H), 4.05 (d, J=5.9 Hz, 2H), 7.68 (dd, J=8.2, 1.6 Hz, 1H), 7.72 (d, J=8.3 Hz, 1H), 7.78 (m, 1H), 7.85 (d, J=1.6 Hz, 1H), 9.34 (br t, J=5.9 Hz, 1H), 9.44 (d, J=1.6 Hz, 1H), 11.28 (s, 1H).

Step 4. Synthesis of N-{2-[2-(3-chlorobenzoyl)hydrazino]-2-oxoethyl}-3-methoxy-4-(4-methyl-1H-imidazol-1-yl)benzamide). N-[3-(Dimethylamino) propyl]-N′-ethylcarbodiimide hydrochloride (EDCI, 333 mg, 1.74 mmol) was added to a solution of N-(2-hydrazino-2-oxoethyl)-3-methoxy-4-(4-methyl-1H-imidazol-1-yl)benzamide, hydrochloride salt (351 mg, 1.03 mmol), 3-chlorobenzoic acid (217 mg, 1.39 mmol) and triethylamine (0.484 mL, 3.47 mmol) in dimethylformamide (10 mL). The reaction mixture was stirred at room temperature for 18 hours, whereupon it was diluted with dichloromethane, and washed with saturated aqueous sodium bicarbonate solution water, and brine. A precipitate formed which was filtered, washed with water and diethyl ether, and dried under vacuum to provide the title compound as a gray powder. Yield: 194 mg, 0.439 mmol, 43%. LCMS m/z 442.6 (M+1). ¹H NMR (500 MHz, DMSO-d₆) δ 2.16 (br s, 3H), 3.91 (s, 3H), 4.03 (d, J=5.9 Hz, 2H), 7.21 (m, 1H), 7.50 (d, J=8.2 Hz, 1H), 7.53 (br d, J=8 Hz, 1H), 7.59-7.64 (m, 2H), 7.72 (d, J=1.7 Hz, 1H), 7.83 (m, 1H), 7.86 (d, J=1.2 Hz, 1H), 7.90 (m, 1H), 9.01 (br t, J=6 Hz, 1H).

Step 5. N-{[5-(3-chlorophenyl)-1,3,4-oxadiazol-2-yl]methyl}-3-methoxy-4-(4-methyl-1H-imidazol-1-yl)benzamide (5). Phosphorus oxychloride (0.38 mL, 3.84 mmol) was added to a solution of N-{2-[2-(3-chlorobenzoyl)hydrazino]-2-oxoethyl}-3-methoxy-4-(4-methyl-1H-imidazol-1-yl)benzamide) (73 mg, 0.16 mmol) in acetonitrile (3 mL), and the mixture was heated at 100° C. for 2 hours. The reaction was allowed to cool to room temperature and then concentrated in vacuo. The residue was dissolved in dichloromethane and washed with saturated aqueous sodium bicarbonate solution, and concentrated under reduced pressure. Chromatography on silica (Eluant: 10% methanol in dichloromethane) afforded the title compound as a gray solid. Yield: 33 mg, 0.078 mmol, 49%. LCMS m/z 424.5, 426.5 (M+1). ¹H NMR (400 MHz, CD₃OD) δ 2.25 (d, J=1.0 Hz, 3H), 3.96 (s, 3H), 4.92 (s, 2H), 7.15 (m, 1H), 7.49 (d, J=8.2 Hz, 1H), 7.57 (dd, J=8.0, 7.7 Hz, 1H), 7.60-7.64 (m, 2H), 7.74 (d, J=1.8 Hz, 1H), 7.89 (d, J=1.4 Hz, 1H), 7.98 (ddd, J=7.7, 1.4, 1.4 Hz, 1H), 8.05 (dd, J=1.8, 1.8 Hz, 1H).

### EXAMPLE 6

### Synthesis of N-[2-(3-chlorophenyl)ethyl]-3-methoxy-4-(3-methyl-[1,2,4]triazol-1-yl)benzamide (6)

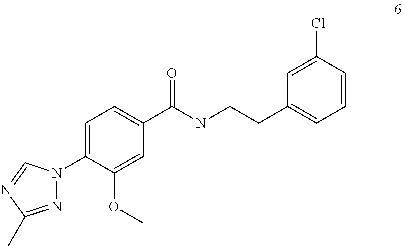

Step 1. Synthesis of 4-hydrazino-3-methoxybenzoic acid methyl ester. To a stirred suspension of 4-amino-3-methoxybenzoic acid methyl ester (62 g, 0.342 mol) in conc. HCl (620 mL) was added drop-wise a solution of NaNO₂ (24.8 g, 0.359 mol) in H₂O (496 mL) at −10° C. After completion of the addition, the reaction mixture was stirred at 0° C. for 1.5 hours. A solution of SnCl₂.2H₂O (247 g, 1.09 mol) in H₂O (248 mL) and conc. HCl (248 mL) was added drop-wise at −10° C. The mixture was stirred at −10° C. for 1.5 hours and the resulting white precipitate was collected by filtration. The solids were suspended in EtOAc (200 mL) and the mixture was basified to pH 12 with a saturated aqueous solution of K₂CO₃ and extracted with EtOAc (200 mL×3). The organic phase was washed with brine (200 mL×3), dried over sodium sulfate and concentrated. The resulting solid was triturated with petroleum ether to afford the title compound, which was used directly in the next step without further purification. Yield: 38.5 g, 196 mmol, 58%. ¹H NMR (400 MHz, DMSO-d₆): δ 3.72 (s, 3H), 3.76 (s, 3H), 4.10 (s, 2H), 6.78 (s, 1H), 6.96 (d, J=8.4 Hz, 1H), 7.22 (d, J=1.6 Hz, 1H), 7.46 (dd, J=8.4, 1.6 Hz, 1H).

Step 2. Synthesis of thioacetimic acid methyl ester. MeI (19.0 mL, 0.306 mol) was added drop-wise to a solution of thioazetamide (10.0 g, 0.133 mol) in acetone (200 mL), at 0° C. The reaction mixture was stirred at room temperature overnight, whereupon the solvent was removed under reduced pressure. The residue was washed with Et₂O and solids were collected by filtration to afford the title compound as a yellow solid, which was used directly in the next step without further purification. Yield: 27.8 g, 312 mmol, 96%.

Step 3. 3-Methoxy-4-(3-methyl[1,2,4]triazol-1-yl)benzoic acid methyl ester. To a suspension of 4-hydrazino-3-methoxybenzoic acid methyl ester (38.5 g, 0.196 mol) in MeOH (385 mL) was added thioacetimic acid methyl ester (42.7 g, 0.196 mol). The reaction mixture was stirred at room temperature for 30 min, whereupon the solvent was removed under reduced pressure. Toluene (385 mL), HC(OMe)₃ (188.7 mL) and pyridine (385 mL) were added, and the reaction mixture was stirred at 100° C. overnight and then concentrated under reduced pressure. The resulting residue was dissolved in a saturated aqueous solution of NaHCO₃, and the mixture was extracted with EtOAc (200 mL×3). The combined organic layers were washed with brine (200 mL×3), dried over sodium sulfate and concentrated. The residue was purified by chromatography on silica (Eluant: petroleum ether/EtOAc=20:1-4:1) to afford the title compound as a yellow solid. Yield: 20.0 g, 81.0 mmol, 41%. ¹H NMR (400 MHz, CDCl₃) δ2.44 (s, 3H), 3.88 (s, 3H), 3.95 (s, 3H), 7.68 (d, J=1.20 Hz, 1H), 7.70 (d, J=8.4, 1.20 Hz, 1H), 7.86 (d, J=8.4 Hz, 1H), 8.76 (s, 1H).

Step 4. 3-Methoxy-4-(3-methyl-[1,2,4]triazol-1-yl)benzoic acid. To a suspension of 3-Methoxy-4-(3-methyl[1,2,4]triazol-1-yl)-benzoic acid methyl ester (20.0 g, 81.0 mmol) in MeOH (300 mL) was added drop wise a solution of KOH (9.08 g, 0.16 mol) in H₂O (100 mL) at 0° C. The reaction mixture was allowed to warm to room temperature and was stirred overnight, whereupon the solvent was removed under reduced pressure. The residue was dissolved in H₂O (300 mL) and washed with EtOAc (100 mL×3). The aqueous phase was acidified to pH 3 with an aqueous solution of citric acid, and the precipitate was collected by filtration to afford the title compound as a gray solid. Yield: 12.5 g, 54.0 mmol, 66%. LCMS m/z 234.0 (M+1). ¹H NMR (400 MHz, DMSO): δ2.32 (s, 3H), 3.93 (s, 3H), 7.63 (dd, J₁=8.0, 1.6 Hz, 1H), 7.68 (br. s 1H), 7.77 (d, J=8.0 Hz, 1H), 8.91 (s, 1H)

Step 5. Synthesis of N-[2-(3-chlorophenyl)ethyl]-3-methoxy-4-(3-methyl-[1,2,4]triazol-1-yl)-benzamide (6). 3-Methoxy-4-(3-methyl-1H-1,2,4-triazol-1-yl)benzoic acid (80 mg, 0.34 mmol) was combined with 2-(3-chlorophenyl)ethanamine (53 mg, 0.34 mmol), 1H-benzotriazol-1-ol (HOBT, 57 mg, 0.41 mmol), diisopropylethylamine (0.24 mL, 0.41 mmol) in dimethylformamide (1.5 mL), and was the mixture was stirred until all solids had dissolved. N-[3-(Dimethylamino)-propyl]-N′-ethylcarbodiimide hydrochloride (EDCI, 83 mg, 0.41 mmol) was added and the reaction was stirred at room temperature overnight. The reaction was diluted with water and CH₂Cl₂, and the layers were separated. The aqueous layer was extracted with CH₂Cl₂, and the combined organic layers were dried over sodium sulfate, filtered and concentrated in vacuo. The residue was purified by silica gel chromatography (Gradient: 3% to 7% [2M ammonia in methanol] in ethyl acetate) to afford the title compound as a solid. Yield: 34 mg, 0.09 mmol, 27%. LCMS m/z 371.1, 373.1 (M+1). ¹H NMR (400 MHz, CDCl₃) δ 2.45 (s, 3H), 2.91 (t, J=6.9 Hz, 2H), 3.65-3.71 (comp, 2H), 3.96 (s, 3H), 6.26 (m, 1H), 7.07-7.12 (m, 1H), 7.19-7.25 (comp, 4H), 7.57 (s, 1H), 7.83 (d, J=8, 4 Hz, 1H), 8.72 (s, 1H).

### EXAMPLE 7

### Synthesis of 3-fluoro-4-(4-methyl-1H-imidazol-1-yl)-N-({4-[3-(trifluoromethyl)phenyl]tetrahydro-2H-pyran-4-yl}methyl)benzamide, formic acid salt (7)

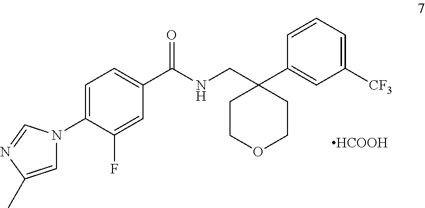

Step 1. Synthesis of 1-{4-[3-(trifluoromethyl)phenyl]tetrahydro-2H-pyran-4-yl}methanamine.

A. Preparation of 4-[3-(trifluoromethyl)phenyl]tetrahydro-2H-pyran-4-carbonitrile. [3-(Trifluoromethyl)phenyl]acetonitrile (40.7 g, 220 mmol) and bis(2-chloroethyl)ether (25.8 mL, 220 mmol) were dissolved in dimethylformamide (800 mL). Sodium hydride (60% suspension in mineral oil, 17.58 g, 440 mmol) was added in small portions over 1.5 hours, such that the temperature of the reaction did not exceed 50-55° C. After completion of the addition, the reaction was stirred at 55° C. for 2 hours, and then stirred at room temperature for 18 hours. Excess sodium hydride was slowly decomposed by drop-wise addition of water until hydrogen evolution ceased. The mixture was diluted with water (2 L), and extracted with ethyl acetate (3×300 mL). The combined extracts were washed with brine, dried over sodium sulfate, and concentrated in vacuo. Chromatography on silica (Eluant: carbon tetrachloride, then 85:15 carbon tetrachloride:ethyl acetate) provided the title compound. Yield: 48 g, 188 mmol, 85%. ¹H NMR (400 MHz, DMSO-d₆) δ 2.08-2.19 (m, 4H), 3.68 (ddd, J=11.5, 11.5, 2.9 Hz, 2H), 4.03 (m, 2H), 7.71 (dd, J=7.8, 7.8 Hz, 1H), 7.77 (br d, J=7.8 Hz, 1H), 7.86 (br s, 1H), 7.90 (br d, J=7.8 Hz, 1H).

B. Synthesis of 1-{4-[3-(trifluoromethyl)phenyl]tetrahydro-2H-pyran-4-yl}methanamine. A solution of 4-[3-(trifluoromethyl)phenyl]tetrahydro-2H-pyran-4-carbonitrile (55.9 g, 219 mmol) in an ammonia/methanol mixture (825 mL) was purged with argon, and Raney Nickel (30 g) was added. The reaction mixture was purged with hydrogen and stirred under a hydrogen balloon at room temperature, until the reaction was complete as monitored by thin layer chromatography (about 24 hours). The reaction mixture was filtered through Celite, and the filtrate was concentrated under reduced pressure. Chromatography on silica (Gradient: 0% to 5% methanol in [chloroform containing 1% diethylamine]) afforded the title compound. Yield: 46.3 g, 179 mmol, 82%. LCMS m/z 260.1 (M+1). ¹H NMR (400 MHz, DMSO-d₆) δ 1.86 (ddd, J=13.7, 8.8, 3.9 Hz, 2H), 2.02 (m, 2H), 2.69 (s, 2H), 3.36 (ddd, J=11.3, 8.6, 2.9 Hz, 2H), 3.68 (ddd, J=11.5, 6.4, 3.9 Hz, 2H), 7.58 (m, 3H), 7.65 (m, 1H).

Step 2. Synthesis of 3,4-difluoro-N-({4-[3-(trifluoromethyl)phenyl]tetrahydro-2H-pyran-4-yl}methyl)benzamide. 1-{4-[3-(Trifluoromethyl)phenyl]tetrahydro-2H-pyran-4-yl}methanamine (1.650 g, 6.36 mmol), 3,4-difluorobenzoic acid (1.0 g, 6.3 mmol), 1H-benzotriazol-1-ol (HOBT, 1.03 g, 7.62 mmol) and diisopropylethylamine (4.42 mL, 25.4 mmol) were combined in dimethylformamide (25 mL), and the mixture was stirred until dissolution was complete. N-[3-(Dimethylamino)propyl]-N′-ethylcarbodiimide hydrochloride (EDCI, 1.46 g, 7.62 mmol) was added, and the reaction was stirred at room temperature for 18 hours, whereupon it was poured into aqueous sodium bicarbonate solution (150 mL) and extracted with ethyl acetate (3×100 mL). The combined organic layers were washed with saturated aqueous sodium bicarbonate solution (60 mL) and brine (60 mL), dried over sodium sulfate and concentrated under reduced pressure. Chromatography on silica (Gradient: 30% to 70% ethyl acetate in heptane) afforded the title compound as a white solid. Yield: 1.98 g, 4.95 mmol, 78%. LCMS m/z 398.2 (M−1). ¹H NMR (400 MHz, CDCl₃) δ 2.01 (half of ABXX′ pattern, J=13.9, 7.6, 3.4 Hz, 2H), 2.14 (half of ABXX′ pattern, J=13.7, 6.7, 3.2 Hz, 2H), 3.62 (ddd, J=11.8, 7.6, 3.2 Hz, 2H), 3.70 (d, J=6.4 Hz, 2H), 3.89 (ddd, J=11.8, 6.8, 3.4 Hz, 2H), 5.64 (br t, J=6 Hz, 1H), 7.16 (m, 1H), 7.26 (m, 1H), 7.46 (ddd, J=10.5, 7.4, 2.0 Hz, 1H), 7.59 (m, 4H). ¹³C NMR (100 MHz, CDCl₃) Partial spectrum: δ 33.44, 41.12, 48.76, 63.82, 116.66 (d, J=18 Hz), 117.51 (d, J=18 Hz), 122.86 (dd, J=7, 4 Hz), 123.26 (q, J=4 Hz), 123.95 (q, J=4 Hz), 129.66, 129.93, 150.14 (dd, J=226, 13 Hz), 152.65 (dd, J=230, 13 Hz), 165.3.

Step 3. Synthesis of 3-fluoro-4-(4-methyl-1H-imidazol-1-yl)-N-({4-[3-(trifluoromethyl)-phenyl]tetrahydro-2H-pyran-4-yl}methyl)benzamide, formic acid salt (7). A mixture of 3,4-difluoro-N-({4-[3-(trifluoromethyl)phenyl]tetrahydro-2H-pyran-4-yl}methyl)benzamide (150 mg, 0.376 mmol), 4-methyl-1H-imidazole (61.7 mg, 0.751 mmol) and potassium carbonate (105 mg, 0.76 mmol) in dimethyl sulfoxide (0.75 mL), was heated at 130° C. for 18 hours. After cooling to room temperature, the reaction was poured into water (30 mL) and extracted with ethyl acetate (3×30 mL). The combined organic layers were washed with brine (40 mL), dried over magnesium sulfate, and concentrated under reduced pressure. Chromatography on silica (Gradient: 30% to 60% [9:1 ethyl acetate: 2M ammonia in methanol] in ethyl acetate), followed by preparative high-pressure liquid chromatography (HPLC) purification (Column: Agilent bonus RP, 5 μm; Mobile phase A: 0.1% formic acid in water; Mobile phase B: 0.1% formic acid in acetonitrile; Gradient: 5% B to 95% B) to afford compound as a gum. Yield: 47 mg, 0.093 mmol, 25%. LCMS m/z 462.2 (M+1). ¹H NMR (400 MHz, CDCl₃) δ 2.03 (m, 2H), 2.17 (m, 2H), 2.30 (s, 3H), 3.63 (m, 2H), 3.72 (d, J=6.4 Hz, 2H), 3.91 (m, 2H), 5.91 (br t, J=6 Hz, 1H), 7.01 (s, 1H), 7.37-7.43 (m, 2H), 7.55-7.61 (m, 5H), 7.89 (s, 1H), 8.20 (s, <1H)

### EXAMPLE 8

### Synthesis of N-[2-(3-chlorophenyl)ethyl]-3-fluoro-4-(4-methyl-1H-imidazol-1-yl)benzamide (8)

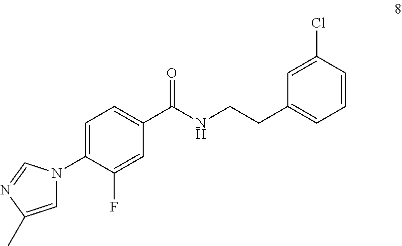

Step 1. Synthesis of 3-fluoro-4-(4-methyl-1H-imidazol-1-yl)benzaldehyde. 4-Methyl-1H-imidazole (1.67 g, 20.3 mmol) and potassium carbonate (3.52 g, 25.5 mmol) were added to a solution of 3,4-difluorobenzaldehyde (2.24 mL, 20.4 mmol) in dimethylformamide (25 mL). The mixture was heated at 110° C. for 18 hours, whereupon it was allowed to cool to room temperature. The reaction was poured into aqueous sodium bicarbonate solution (150 mL) and extracted with ethyl acetate (3×100 mL). The combined organic layers were washed with saturated aqueous sodium bicarbonate solution (60 mL) and brine (60 mL), dried over magnesium sulfate and concentrated under reduced pressure provided a residue. Chromatography on silica (Gradient: 70% to 100% ethyl acetate in heptane) provided the title compound as a white solid. Yield: 88 mg, 0.43 mmol, 2%. ¹H NMR (400 MHz, CDCl₃) δ 2.30 (d, J=1 Hz, 3H), 7.07 (m, 1H), 7.56 (dd, J=7.6, 7.6 Hz, 1H), 7.76-7.79 (m, 2H), 7.85 (m, 1H), 9.99 (d, J=1.9 Hz, 1H).

Step 2. Synthesis of N-[2-(3-chlorophenyl)ethyl]-3-fluoro-4-(4-methyl-1H-imidazol-1-yl)benzamide (8). 3-Fluoro-4-(4-methyl-1H-imidazol-1-yl)benzaldehyde (88 mg, 0.43 mmol) was added to a solution of potassium hydroxide (85%, 114 mg, 1.7 mmol) in methanol (0.71 mL) and water (0.12 mL), and the resulting solution was heated to 65° C., whereupon aqueous hydrogen peroxide (30%, 0.35 mL, 3.4 mmol) was added drop-wise over 20 minutes. Following completion of the addition, the reaction was stirred for an additional 25 minutes at 65° C., whereupon it was allowed to cool to room temperature and acidified with concentrated hydrochloric acid. Isolation of the product by filtration at this stage was unsuccessful, so the mixture was diluted with water (10 mL) and the pH was adjusted to 4 with 1N aqueous sodium hydroxide solution. This was concentrated in vacuo to give a mixture of 3-fluoro-4-(4-methyl-1H-imidazol-1-yl)benzoic acid and inorganic salts as a white solid. LCMS m/z 221.2 (M+1). This mixture was combined in dimethylformamide (4.3 mL) with 2-(3-chlorophenyl)ethanamine (0.181 mL, 1.29 mmol), 1H-benzotriazol-1-ol (HOBT, 174 mg, 1.29 mmol) and diisopropylethylamine (0.524 mL, 3.01 mmol). N-[3-(Dimethylamino)propyl]-N′-ethylcarbodiimide hydrochloride (EDCI, 247 mg, 1.29 mmol) was added, and the reaction was stirred for 66 hours, whereupon it was poured into water (50 mL) and extracted with ethyl acetate (3×30 mL). The combined organic layers were washed with saturated aqueous sodium bicarbonate solution (50 mL), and brine (50 mL), dried over magnesium sulfate and concentrated under reduced pressure. Chromatography on silica (Mobile phase A: ethyl acetate; Mobile phase B: 9:1 ethyl acetate/[2M ammonia in methanol]; Gradient: 0% B to 50% B) to provide compound the title compound as a solid. Yield: 90 mg, 0.25 mmol, 58%. LCMS m/z 358.1 (M+1). ¹H NMR (400 MHz, CDCl₃) δ 2.31 (s, 3H), 2.94 (t, J=6.9 Hz, 2H), 3.73 (dt, apparent q, J=6.2, 6.7 Hz, 2H), 6.25 (br s, 1H), 7.02 (br s, 1H), 7.13 (br d, J=6.8 Hz, 1H), 7.24-7.28 (m, 3H), 7.42 (dd, J=7.8, 7.8 Hz, 1H), 7.55 (br d, J=8.3 Hz, 1H), 7.66 (dd, J=11.4, 1.9 Hz, 1H), 7.78 (br s, 1H).

Cell-Based γ-Secretase Assay with ELISA Readout

The ability of compounds to modulate production of amyloid beta protein Aβ(1-42) was determined using human WT-APP overexpressing CHO cells. Cells were plated at 22,000 cells/100 μL well in 96 well tissue culture treated, clear plates (Falcon) in DMEM/F12 based medium and incubated for 24 hours at 37° C. Compounds for testing were diluted in 100% DMSO to achieve an eleven points, half log, dose response for IC₅₀ determinations. Compounds were added in fresh medium to achieve 1% final DMSO. Appropriate vehicle and inhibitor controls were added to obtain maximum and minimum inhibition values for the assay before the plates were incubated for about 24 hours at 37° C.

Coating of ELISA assay plates was initiated by addition of 50 μL/well of an in house Aβ(1-42) specific antibody at (4 μg/mL) in 0.1 M NaHCO₃ (pH 9.0) into black 384-well Maxisorp® plates (Nunc) and incubated overnight at 4° C. The capture antibody was then aspirated from the ELISA assay plates and 100 μL/well of Blocking Buffer (Dulbecco's PBS, 1.5% BSA (Sigma A7030)) added. Ambient temperature incubation was allowed to proceed for a minimum of two hours before washing 2×100 μL with Wash Buffer (Dulbecco's PBS, 0.05% Tween 20). Assay Buffer (Dulbecco's PBS, 1.0% BSA (Sigma A7030), 0.05% Tween 20) 20 μL/well is then added.

After incubation overnight at 37° C., 5% CO₂, 40 μL (in duplicate) of experimental conditioned media are transferred into wells of the blocked ELISA plates containing the capture antibody, followed by overnight incubation at 4° C. Cell toxicity was measured in the corresponding cells after removal of the media for the Aβ(1-42) assay by a colorimetric cell proliferation assay (CellTiter 96® AQᵤₑₒᵤₛ One Solution Cell Proliferation Assay, Promega) according to the manufacturer's instructions.

After overnight incubation of the ELISA assay plates at 4° C., unbound Aβ peptides were removed thorough (4×100 μL) washes with Wash Buffer. Europium (Eu) labeled (custom labeled, Perkin Elmer) Aβ(1-16) 6e10 Monoclonal Antibody (Covance #SIG-39320 was added, (50 μL/well Eu-6e10 @ 1:5000, 20 uM EDTA) in Assay Buffer. Incubation at ambient temperature for a minimum of 2 hours was followed by (4×100 μL) washes with Wash Buffer, before 50 μL/well of Delfia Enhancement Solution (Perkin Elmer) was added. Following a one hour ambient temperature incubation the plates were read on an Envision plate reader (Perkin Elmer) using standard DELFIA TRF settings. Data analysis including inhibitory IC₅₀ determination was performed using nonlinear regression fit analysis (in house software) and the appropriate plate mean values for the maximum and minimum inhibition controls.

TABLE 1
Biological data for examples 1-8

| | GM Aβ 42B IC₅₀ (μM) |
|---|---|
| | (Geometric Mean of |
| Example | 2-8 Determinations) |
| 1 | 2.92 |
| 2 | 0.942 |
| 3 | 0.341 |
| 4 | 0.33 |
| 5 | 1.46 |
| 6 | 4.21 |
| 7 | 2.19 |
| 8 | 0.96 |

The compounds in Table 2 were prepared by methods analogous to those described for compounds I-8. The amine coupling partners used in the synthesis of the compounds in Table 2 are either commercially available or known in the literature, or can be prepared by methods known to those skilled in the art.

Synthesis of 5-chloro-2,3-dihydro-1-benzofuran-3-amine enantiomer 1 and 2.

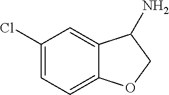

Step 1. Synthesis of methyl 5-chloro-2-hydroxybenzoate. Concentrated sulfuric acid (20 mL) was added to a suspension of 5-chlorosalicylic acid (50 g, 290 mmol) in methanol (500 mL), and the mixture was refluxed for five days. The reaction was concentrated under reduced pressure and the residue was dissolved in Et₂O (500 mL). The resulting mixture was poured into a saturated aqueous solution of NaHCO₃ (400 mL) cooled to 0° C., and the layers were separated. The aqueous layer was extracted with Et₂O (2×400 mL) and the combined organic layers were washed with a saturated aqueous solution of NaHCO₃ and brine. The organic layer was dried (Na₂SO₄) and concentrated under reduced pressure to afford the title compound as a white solid. Yield: 49.5 g, 265 mmol, 91%.

Step 2. Synthesis of methyl 5-chloro-2-(2-ethoxy-2-oxoethoxy)benzoate. Ethyl bromoacetate (30 mL, 265 mmol) was added to a suspension of methyl 5-chloro-2-hydroxybenzoate (49.5 g, 265 mmol) and K₂CO₃ (128 g, 929 mmol) in acetone (1.0 L). The mixture was refluxed overnight, whereupon the reaction was allowed to cool to room temperature and filtered. The filtrate was concentrated under reduced pressure and the residue was dissolved in CH₂Cl₂. The resulting solution was washed twice with water, dried (Na₂SO₄), and concentrated under reduced pressure to afford the title compound as a red wax. Yield: 55 g, 202 mmol, 76%.

Step 3. Synthesis of 5-chloro-1-benzofuran-3(2H)-one. KOt-Bu (48.1 g, 429 mmol) was added in portions to a solution of methyl 5-chloro-2-(2-ethoxy-2-oxoethoxy)benzoate (46.8 g, 171 mmol) in THF (2 L) at 0° C. The mixture was stirred at 0° C. for 2 h, whereupon a saturated aqueous solution of NH₄Cl (500 mL) was added followed by EtOAc (500 mL). The layers were separated and the aqueous layer was extracted with EtOAc (2×1 L). The combined organic layers were washed with brine, dried over Na₂SO₄, and concentrated under reduced pressure to give a mixture of the title compound and ethyl 5-chloro-3-hydroxy-1-benzofuran-2-carboxylate. This mixture was dissolved in DMSO (260 mL) and water (450 mL) and LiOH.H₂O (33.0 g, 803 mmol) was added. The reaction was stirred at 70° C. for 3 hours and then at room temperature overnight. The mixture was poured into a 10% aqueous solution of HCl (1 L) resulting in the formation of a solid precipitate that was collected by filtration and washed with water. The solid material was dissolved in Et₂O and washed with water. The combined organic layers were washed with brine, dried over Na₂SO₄, and concentrated under reduced pressure. Chromatography on silica (Gradient: 0% to 40% ethyl acetate in heptane) provided the title compound as a red solid. Yield: 19.6 g, 117 mmol, 68% over 2 steps.

Step 4. Synthesis of 5-chloro-2,3-dihydro-1-benzofuran-3-ol. NaBH₄ (1.68 g, 44 mmol) was added to a solution of 5-chloro-1-benzofuran-3(2H)-one (9.93 g, 59.1 mmol) in MeOH (600 mL) at 0° C. The mixture was stirred at 0° C. for 2 h and at room temperature for 2 h, whereupon water (500 mL) was added. The reaction mixture was concentrated under reduced pressure to remove most of the MeOH. EtOAc (800 mL) was added and the layers were separated. The aqueous layer was extracted with EtOAc (800 mL), and the combined organic layers were washed with brine, dried over Na₂SO₄, and concentrated under reduced pressure to give the title compound as a red wax. Yield: 9.75 g, 57 mmol, 97%.

Step 5. Synthesis of 3-azido-5-chloro-2,3-dihydro-1-benzofuran. To a solution of 5-chloro-2,3-dihydro-1-benzofuran-3-ol (9.75 g, 57 mmol) in toluene (200 mL) at 0° C. was added 1,8-diazabicyclo[5.4.0]undec-7-ene (DBU) (10.2 mL, 68.4 mmol) followed by diphenylphosphoryl azide (DPPA) (14.8 mL, 68.4 mmol). The mixture was stirred at 0° C. for 3 h and then at room temperature overnight. ¹H NMR indicated 85% conversion of the starting material. The mixture was cooled to 0° C. and additional DBU (2.56 mL, 17.1 mmol) was added followed by DPPA (3.7 mL, 17.1 mmol). The reaction was stirred at 0° C. for 1 h, whereupon ¹H NMR showed that the reaction had reached completion. Water (90 mL) was added to the reaction mixture followed by an aqueous solution of HCl (1 N, 90 mL). The layers were separated and the aqueous layer was extracted three times with CH₂Cl₂. The combined organic layers were dried over Na₂SO₄ and concentrated under reduced pressure. Chromatography on silica (Gradient: 0% to 10% EtOAc in heptane) provided the title compound as a yellow oil. Yield: 6.1 g, 31.3 mmol, 55%.

Step 6. Synthesis of 5-chloro-2,3-dihydro-1-benzofuran-3-amine hydrochloride salt. To a solution of 3-azido-5-chloro-2,3-dihydrobenzofuran (6.10 g, 31.3 mmol) in THF (260 mL) were sequentially added water (5.63 mL) and triphenylphosphine (24.7 g, 94 mmol). The reaction was stirred at 50° C. overnight, whereupon it was allowed to cool to room temperature and diluted with Et₂O (500 mL). HCl (4 N) in dioxane (8.25 mL, 33 mmol) was added and the solution was stirred for 5 min at room temperature, whereupon the precipitate was collected by filtration to afford the title compound as a white solid. Yield=5.9 g, 28.9 mmol, 92%.

Step 7. Synthesis of 5-chloro-2,3-dihydro-1-benzofuran-3-amine. 5-Chloro-2,3-dihydro-1-benzofuran-3-amine hydrochloride salt (10.8 g, 53 mmol) was dissolved in saturated aqueous NaHCO₃ solution (300 mL). The pH was adjusted to 9 by the addition of aqueous NaOH solution (3 N), and the mixture was extracted with CH₂Cl₂/MeOH (90/10) and CHCl₃/MeOH (90/10). The combined organic layers were dried over MgSO₄ and concentrated under reduced pressure to give the title compound. Yield: 5.00 g, 29.6 mmol, 56%. ¹H NMR of the aqueous layer indicated the presence of additional product. The aqueous layer was concentrated to dryness and the residue was stirred in CHCl₃/MeOH (80/20) overnight. The mixture was filtered and the filtrate was concentrated under reduced pressure to furnish additional title compound (0.50 g, 2.96 mmol, 5%). ¹H NMR of the MgSO₄ pad indicated the presence of a significant amount of the desired product. The solids were suspended in a mixture of isopropanol (420 mL) and a 7 N solution of ammonia in MeOH (7 mL) and stirred for 15 minutes. The solids were removed by filtration and the filtrate was concentrated under reduced pressure to afford an additional 3.24 g (19.2 mmol, 36%) of the title compound. The title compound was obtained as a white solid. Combined yield=8.74 g, 52 mmol, 98%.

Step 8. Synthesis of 5-chloro-2,3-dihydro-1-benzofuran-3-amine enantiomer-1. Racemic 5-chloro-2,3-dihydro-1-benzofuran-3-amine (8.74 g, 51.7 mmol) and (+)-phencyphos(2-hydroxy-5,5-dimethyl-4-phenyl-1,3,2-dioxaphosphorinan-2-one) (12.52 g, 51.7 mmol) were suspended in EtOH (300 mL) and water (2 mL). The mixture was heated to reflux using a heat gun and then allowed to cool slowly to room temperature overnight. The resulting solid was isolated by filtration and recrystallized from EtOH/water (120 mL/0.7 mL). The solids were dissolved in aqueous NaOH (3 N, 70 mL) and CH₂Cl₂ (100 mL) and stirred at room temperature for 2 h, whereupon the mixture was filtered to remove the (+)-phencyphos sodium salt. The solids were washed with CH₂Cl₂ and the two layers from the combined filtrate and washings were separated. The aqueous layer was extracted with CH₂Cl₂ and the combined organic layers were dried by adding Na₂SO₄ and stirring for 10 min followed by filtration through a pad of Na₂SO₄ to afford the title compound as a yellow oil. Yield=2.92 g, 17.3 mmol, 33%, ee=96%. The spectral data was identical to that of enantiomer 2 in Step 9.

Step 9. Synthesis of 5-chloro-2,3-dihydro-1-benzofuran-3-amine enantiomer 2. The mother liquor from step 8 was concentrated under reduced pressure to afford 11.65 g of the (+)-phencyphos salt of 5-chloro-2,3-dihydro-1-benzofuran-3-amine enantiomer 2 (28.3 mmol, ee=59%). The solid was dissolved in a mixture of sec-butanol (200 mL) and aqueous KOH solution (1 M, 100 mL) and the layers were separated. To the organic layer (containing the 5-chloro-2,3-dihydro-1-benzofuran-3-amine enantiomer 2 free base, ee=59%) was added (−)-phencyphos (6.78 g, 28 mmol) and the mixture was concentrated under reduced pressure. To the solid was added EtOH (150 mL) and the mixture was heated to reflux using a heat gun and then allowed to cool slowly to room temperature overnight. The resulting solid was isolated by filtration (ee=42%) and the filtrate was concentrated under reduced pressure to afford the (−)-phencyphos salt of 5-chloro-2,3-dihydro-1-benzofuran-3-amine enantiomer 2, 7.16 g, 17.3 mmol, ee=80%. Two recrystallizations from EtOH increased the ee to 85%. The resulting solid was dissolved in aqueous NaOH solution (3 N, 60 mL) and CH₂Cl₂ (100 mL) and the mixture was stirred at room temperature for 2 h, whereupon it was filtered. The solids were rinsed with CH₂Cl₂, and the two layers from the filtrate and washings were separated. The aqueous layer was further extracted with CH₂Cl₂ and the combined organic layers were dried by adding Na₂SO₄ and stirring for 10 min followed by filtration through a pad of Na₂SO₄ to remove the remaining (−)-phencyphos sodium salt. The filtrate was concentrated under reduced pressure to afford the title compound. Yield=1.47 g, 8.7 mmol, ee=85%. To this material was added (−)-phencyphos (2.1 g, 8.7 mmol) and EtOH (40 mL) and water (0.1 mL). The mixture heated to reflux by using a heat gun and then allowed to cool slowly to room temperature overnight. The resulting solid was isolated by filtration (ee=97%). Another batch of 5-chloro-2,3-dihydrobenzofuran-3-ylamine enantiomer 2 (ee=42% 2.72 g, 16 mmol, from the previous unsuccessful recrystallization) and (−)-phencyphos (3.87 g, 16 mmol) were suspended in EtOH (70 mL) and water (0.3 mL). The mixture was heated to reflux using a heat gun and then allowed to cool slowly to room temperature overnight. The resulting solid was isolated by filtration (ee=93%) and recrystallized from EtOH/water (35 mL/0.5 mL) and again isolated by filtration (ee=97%). The two batches of the 5-chloro-2,3-dihydro-1-benzofuran-3-amine enantiomer-2 (−)-phencyphos salt were combined (ee=97%, 7.3 g, 17.8 mmol) and dissolved in NaOH solution (3 N, 80 mL) and CH₂Cl₂ (100 mL). The mixture was stirred at room temperature for 2 h, whereupon it was filtered to remove the (−)-phencyphos sodium salt. The solids were rinsed with CH₂Cl₂, and the two layers from the filtrate and washings were separated. The aqueous layer was further extracted with CH₂Cl₂ and the combined organic layers were dried by adding Na₂SO₄ and stirring for 10 min followed by filtration through a pad of Na₂SO₄ to remove the remaining (−)-phencyphos sodium salt. The filtrate was concentrated under reduced pressure to afford the title compound as a pale yellow oil. Yield=3 g, 17.8 mmol, 34%, ee>97%. The absolute configuration was not determined. LCMS m/z 153.0 [(M-NH₃)+1]. ¹H NMR (400 MHz, CDCl₃) δ 4.16 (dd, J=9.2, 4.7 Hz, 1H) 4.56-4.68 (m, 2H) 6.71 (d, J=8.6 Hz, 1H) 7.11 (dd, J=8.4, 2.2 Hz, 1H) 7.24 (br s, 1H).

### Synthesis of 5-(trifluoromethyl)-2,3-dihydro-1-benzofuran-3-amine

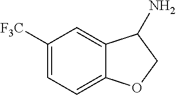

Step 1. Synthesis of N-{(1E)-[2-hydroxy-5-(trifluoromethyl)phenyl]methylene}-2-methylpropane-2-sulfinamide. Cs₂CO₃ (6.23 g, 19.1 mmol) was added to a solution of 2-hydroxy-5-(trifluoromethyl)benzaldehyde (1.65 g, 8.68 mmol) and tert-butylsulfinamide (2.17 g, 17.4 mmol) in CH₂Cl₂ (87 mL). The reaction mixture was heated to reflux overnight and was then allowed to cool to room temperature. The mixture was filtered through celite, and the filtrate was concentrated under reduced pressure. Chromatography on silica (Gradient: 10% to 80% EtOAc in heptane) furnished the title compound as a white solid. Yield: 1.59 g, 5.42 mmol, 62%. LCMS m/z 294.2 (M+1). ¹H NMR (400 MHz, CDCl₃) δ 1.25 (s, 9H), 7.10 (d, J=8.8 Hz, 1H), 7.65 (dd, J=8.6, 2.2 Hz, 1H), 7.75 (d, J=2.0 Hz, 1H), 8.71 (s, 1H), 11.43 (s, 1H).

Step 2. Synthesis of 2-methyl-N-[5-(trifluoromethyl)-2,3-dihydro-1-benzofuran-3-yl]propane-2-sulfinamide. KOt-Bu (608 mg, 5.42 mmol) was added to a solution of N-{(1E)-[2-hydroxy-5-(trifluoromethyl)phenyl]methylene}-2-methylpropane-2-sulfinamide (1.59 g, 5.42 mmol) and trimethylsulfoxonium iodide (1.19 g, 5.42 mmol) in DMSO (27 mL). The reaction was stirred at room temperature overnight whereupon it was poured into water cooled to 0° C. The mixture was extracted three times with EtOAc and the combined organic layers were dried over MgSO₄ and concentrated under reduced pressure. Chromatography on silica (Gradient: 30% to 80% EtOAc in heptane) afforded the title compound. Yield: 100 mg, 0.33 mmol, 6%. LCMS m/z 308.1 (M+1). ¹H NMR (400 MHz, CDCl₃, mixture of diastereomers) δ 1.21 (s, 3.6; H), 1.23 (s, 5.4; H), 3.46-3.60 (m, 1H), 4.46 (dd, J=10.2, 4.5 Hz, 0.4; H), 4.59 (dd, J=10.5, 4.9 Hz, 0.6; H), 4.72 (dd, J=10.2, 8.2 Hz, 0.4; H), 4.80 (dd, J=10.5, 8.2 Hz, 0.6; H), 5.07-5.22 (comp, 1H), 6.88 (d, J=8.8 Hz, 0.4; H), 6.90 (d, J=8.6 Hz, 0.6; H), 7.47-7.52 (comp, 1H), 7.54 (s, 0.6; H), 7.75 (s, 0.4H).

Step 3. Synthesis of 5-(trifluoromethyl)-2,3-dihydro-1-benzofuran-3-amine. 4 M HCl in dioxane (0.24 mL, 0.96 mmol) was added to a solution of 2-methyl-N-[5-(trifluoromethyl)-2,3-dihydro-1-benzofuran-3-yl]propane-2-sulfinamide (100 mg, 0.33 mmol) in MeOH (2.5 mL). The reaction was stirred at room temperature for 1.5 h, whereupon the reaction was concentrated under reduced pressure to afford the crude title compound as a white solid. GCMS m/z 187 (M-NH₂). The crude material was used directly in the ensuing amide coupling reaction without further purification.

TABLE 2

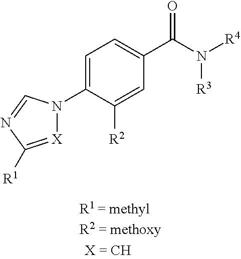

| | | | | Mass spec: | ¹H NMR (400 MHz, |
|---|---|---|---|---|---|
| | | Aβ 42 | | observed | CDCl₃ unless otherwise |
| | | IC₅₀ | IUPAC | ion m/z | specified); observed |
| Ex# | Structure of NR³R⁴ | (μM)^{*} | Name | (M + 1) | peaks, δ (ppm) |
| 9 | 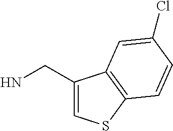 | 0.158 | N-[(5-chloro- 1-benzothien- 3-yl)methyl]- 3-methoxy-4- (4-methyl-1H- imidazol-1- yl)benzamide | 412.3, LCMS | (500 MHz, CDCl₃) 2.24 (br s, 3H), 3.85 (s, 3H), 4.83 (d, J = 5.6 Hz, 2H), 6.91 (m, 1H), 7.20 (d, J = 8.2 Hz, 1H), 7.29 (br t, J = 5.6 Hz, 1H), 7.31 (dd, J = 8.6, 2.0 Hz, 1H), 7.36 (dd, J = 8.1, 1.7 Hz, 1H), 7.44 (br s, 1H), 7.62 (d, J = 1.7 Hz, 1H), 7.66 (d, J = 1.2 Hz, 1H), 7.75 (d, J = 8.5 Hz, 1H), 7.83 (d, J = 2.0 Hz, 1H). |
| 10 | 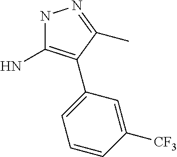 | 0.245 | 3-methoxy-4- (4-methyl-1H- imidazol-1-yl)- N-{3-methyl- 4-[3-(trifluoro- methyl)phenyl]- 1H-pyrazol-5- yl}benzamide | 456.1, LCMS | (500 MHz, CD₃OD) 2.41 (s, 3H), 2.43 (d, J = 1.0 Hz, 3H), 3.98 (s, 3H), 7.54-7.67 (m, 7H), 7.70 (br s, 1H), 7.76 (br s, 1H), 9.17 (d, J = 1.5 Hz, 1H). |
| 11 | 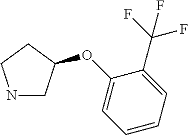 | 0.096 | 1-[2-methoxy- 4-({(3R)-3-[2- (trifluoromethyl) phenoxy] pyrrolidin-1- yl}carbonyl) phenyl]-4- methyl-1H- imidazole | 446.3, LCMS | (CD₃OD, two rotamers) 2.27 and 2.28 (2 br s, 3H), 2.27-2.37 (m, 2H), 3.62-3.91 (m, 4H), 3.87 and 3.94 (2 s, 3H), 5.22 and 5.34 (2 br m, 1H), 7.05- 7.63 (m, 7H), 8.06 and 8.10 (2 m, 1H), 8.17 (br s, 1H). |
| 12 | 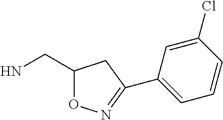 | 1.31 | N-{[3-(3- chlorophenyl)- 4,5-dihydro- isoxazol-5-yl] methyl}-3- methoxy-4- (4-methyl-1H- imidazol-1-yl) benzamide | 425.6, LCMS | (CD₃OD) 2.43 (d, J = 1.0 Hz, 3H), 3.31 (dd, J = 17.1, 6.7 Hz, 1H, assumed, partially obscured by solvent peak), 3.55 (dd, J = 17.2, 10.7 Hz, 1H), 3.67 (m, 2H), 3.95 (s, 3H), 5.03 (m, 1H), 7.39- 7.45 (m, 2H), 7.57- 7.62 (m, 4H), 7.70 (m, 2H), 9.18 (d, J = 1.6 Hz, 1H). |
| 13 | 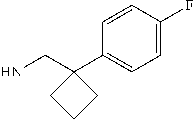 | 0.261 ** | N-{[1-(4- fluorophenyl) cyclobutyl] methyl}-3- methoxy-4-(4- methyl-1H- imidazol-1-yl) benzamide | 394.2, LCMS | 1.90-1.99 (m, 1H), 2.24-2.40 (m, 5H), 2.29 (br s, 3H), 3.83 (d, J = 6.0 Hz, 2H), 3.90 (s, 3H), 5.86 (m, 1H), 6.93 (m, 1H), 7.04-7.10 (m, 3H), 7.13-7.17 (m, 2H), 7.25 (d, J = 8.1 Hz, 1H), 7.51 (br s, 1H), 7.73 (br s, 1H). |
| 14 | 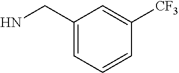 | 0.447 | 3-methoxy-4- (4-methyl-1H- imidazol-1-yl)- N-[3-(trifluoro- methyl)benzyl] benzamide | 390.1, LCMS | 2.29 (d, J = 1.0 Hz, 3H), 3.93 (s, 3H), 4.74 (d, J = 5.9 Hz, 2H), 6.78 (br t, J = 5.6 Hz, 1H), 6.96 (m, 1H), 7.30 (d, J = 8.0 Hz, 1H), 7.36 (dd, J = 8.1, 1.8 Hz, 1H), 7.49 (m, 1H), 7.57-7.62 (m, 3H), 7.65 (d, J = 1.8 Hz, 1H), 7.74 (d, J = 1.3 Hz, 1H). |
| 15 | 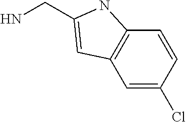 | 0.547 | N-[(5-chloro- 1H-indol-2-yl) methyl]-3- methoxy-4-(4- methyl-1H- imidazol-1- yl)benzamide | 395.2, LCMS | 2.28 (br s, 3H), 3.89 (s, 3H), 4.71 (d, J = 5.9 Hz, 2H), 6.33 (s, 1H), 6.95 (m, 1H), 7.11 (m, 1H), 7.22-7.27 (m, 2H), 7.33-7.37 (m, 2H), 7.51 (br s, 1H), 7.61 (br s, 1H), 7.69 (br s, 1H), 9.40 (br s, 1H). |
| 16 | 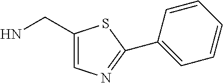 | 0.283 | 3-methoxy- 4-(4-methyl- 1H-imidazol- 1-yl)-N-[(2- phenyl-1,3- thiazol-5- yl)methyl] benzamide | 405.1, LCMS | 2.28 (br s, 3H), 3.91 (s, 3H), 4.86 (d, J = 5.7 Hz, 2H), 6.95 (m, 1H), 7.08 (br t, J = 5.6 Hz, 1H), 7.28 (d, 1H, assumed, obscured by solvent), 7.37 (dd, J = 8.2, 1.8 Hz, 1H), 7.41-7.44 (m, 3H), 7.65 (d, J = 1.8 Hz, 1H), 7.73 (m, 2H), 7.91 (m, 2H). |
| 17 | 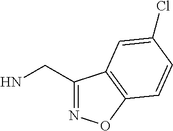 | 0.371 | N-[(5-chloro- 1,2-benz- isoxazol-3-yl) methyl]-3- methoxy-4-(4- methyl-1H- imidazol-1-yl) benzamide | 397.1, 399.1, LCMS | (CD₃OD) 2.24 (d, J = 1.0 Hz, 3H), 3.95 (s, 3H), 4.98 (s, 2H), 7.13 (m, 1H), 7.47 (d, J = 8.1 Hz, 1H), 7.58 (dd, J = 8.1, 1.8 Hz, 1H), 7.60 (dd, half of ABX system, J = 8.9, 1.9 Hz, 1H), 7.64 (dd, half of ABX system, J = 8.9, 0.8 Hz, 1H), 7.70 (d, J = 1.9 Hz, 1H), 7.87 (d, J = 1.4 Hz, 1H), 7.97 (dd, J = 1.9, 0.8 Hz, 1H). |
| 18 | 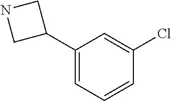 | 0.66 | 1-(4-{[3-(3- chlorophenyl) azetidin-1-yl] carbonyl}-2- methoxy- phenyl)-4- methyl-1H- imidazole | 382.1, LCMS | 2.29 (d, J = 1.0 Hz, 3H), 3.86-3.94 (m, 1H), 3.91 (s, 3H), 4.25-4.36 (br m, 2H), 4.59-4.75 (br m, 2H), 6.94 (m, 1H), 7.20-7.32 (m, 6H), 7.46 (d, J = 1.5 Hz, 1H), 7.75 (br s, 1H). |
| 19 | 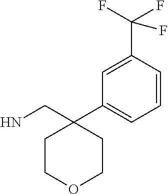 | 0.757 | 3-methoxy-4- (4-methyl-1H- imidazol-1-yl)- N-({4-[3-(tri- fluoromethyl) phenyl] tetrahydro- 2H-pyran- 4-yl}methyl) benzamide | 474.2, LCMS | 2.05 (m, 2H), 2.16 (m, 2H), 2.27 (s, 3H), 3.65 (m, 2H), 3.74 (d, J = 6.4 Hz, 2H), 3.88 (s, 3H), 3.92 (m, 2H), 5.86 (br t, J = 6.4 Hz, 1H), 6.93 (br s, 1H), 7.02 (dd, J = 8.1, 1.9 Hz, 1H), 7.22 (d, J = 8.1 Hz, 1H), 7.46 (d, J = 1.7 Hz, 1H), 7.60 (m, 4H), 7.70 (br s, 1H) |
| 20 | 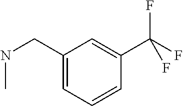 | 0.997 | 3-methoxy- N-methyl-4- (4-methyl-1H- imidazol-1-yl)- N-[3-(trifluoro- methyl)benzyl] benzamide | 404.1, LCMS | 2.29 (s, 3H), 2.98 and 3.09 (2 br s, 3H), 3.74 and 3.89 (2 br s, 3H), 4.63 and 4.81 (2 br s, 2H), 6.92 (br s, 1H), 7.08 (br d, J = 8 Hz, 1H), 7.16 (br s, 1H), 7.28 (br s, 1H), 7.44 (br s, 1H), 7.51-7.60 (m, 3H), 7.70 (br s, 1H) |
| 21 | 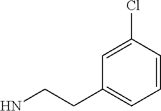 | 0.629 | N-[2-(3-chloro- phenyl)ethyl]- 3-methoxy-4- (4-methyl-1H- imidazol-1-yl) benzamide | 370.0, 372.0, LCMS | 2.31 (s, 3H), 2.94 (t, J = 7.0 Hz, 2H), 3.71 (dt, apparent q, J = 6.6, 6.6 Hz, 2H), 3.90 (s, 3H), 6.78 (br t, J = 5.7 Hz, 1H), 6.96 (s, 1H), 7.13 (br d, J = 7.0 Hz, 1H), 7.21-7.31 (m, 5H), 7.56 (br s, 1H), 8.03 (br s, 1H), 8.32 (s, 1H). |
| 22 | 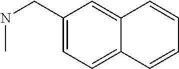 | 0.977 | 3-methoxy-N- methyl-4-(4- methyl-1H- imidazol-1-yl)- N-(2-naphthyl- methyl) benzamide | 386.2, LCMS | rotamers evident; complex NMR, d 2.24 and 2.28 (2 br s, 3H), 2.82 and 3.22 (2 br s, 3H), 3.51 and 3.85 (2 br s, 3H), 5.03 and 5.24 (2 br s, 2H), 6.84-6.91 (m, 1H), 6.99-7.23 (m, 3H), 7.46-7.56 (m, 4H), 7.63 (m, partial H), 7.70 (br s, 1H), 7.85 (m, 1H), 7.90 (m, 1H), 8.18 (m, partial H) |
| 23 | 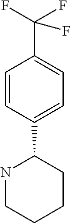 | 0.838 | (2S)-1-[3- methoxy-4- (4-methyl-1H- imidazol-1- yl)benzoyl]-2- [4-(trifluoro- methyl)phenyl] piperidine | 444.1, LCMS | 1.65 (br s, 3H), 1.78 (br m, 1H), 2.04 (br s, 2H), 2.30 (s, 3H), 2.47 (br m, 1H), 2.95 (br s, 1H), 3.85 (br s, 3H), 4.45-6.3 (very broad humps, assumed 1H), 6.92 (br s, 1H), 7.07- 7.15 (m, 2H), 7.27 (br s, 1H), 7.47 (d, J = 8.2 Hz, 2H), 7.68 (d, J = 8.2 Hz, 2H), 7.71 (br s, 1H) |
| 24 | 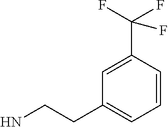 | 0.438 | 3-methoxy-4- (4-methyl-1H- imidazol-1-yl)- N-{2-[3-(tri- fluoromethyl) phenyl]ethyl} benzamide | 404.2, LCMS | 2.27 (s, 3H), 3.03 (t, J = 7.0 Hz, 2H), 3.74 (dt, apparent q, J = 6.6, 6.6 Hz, 2H), 3.88 (s, 3H), 6.64 (br t, J = 5.9 Hz, 1H), 6.94 (br s, 1H), 7.24 (br s, 2H), 7.44 (m, 2H), 7.50 (m, 2H), 7.55 (s, 1H), 7.68 (s, 1H) |
| 25 | 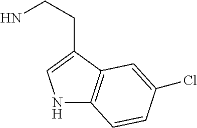 | 0.195 | N-[2-(5-chloro- 1H-indol- 3-yl)ethyl]-3- methoxy-4-(4- methyl-1H- imidazol-1-yl) benzamide | 409.1, 411.1, LCMS | 2.27 (s, 3H), 3.06 (t, J = 6.6, 2H), 3.76 (m, 2H), 3.85 (s, 3H), 6.26 (br s, 1H), 6.91 (m, 1H), 7.09-7.17 (m, 3H), 7.18-7.30 (m, 2H), 7.49 (br s, 1H), 7.57 (br s, 1H), 7.69 (br s, 1H), 8.35 (br s, 1H). |
| 26 | 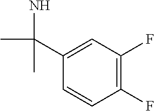 | 0.787 | N-[1-(3,4- difluoro- phenyl)-1- methylethyl]- 3-methoxy-4- (4-methyl-1H- imidazol-1-yl) benzamide | 386.1, LCMS | 1.79 (s, 6H), 2.30 (s, 3H), 3.89 (s, 3H), 6.59 (br s, 1H), 6.95 (m, 1H), 7.09-7.19 (m, 2H), 7.22-7.33 (m, 3H), 7.57 (br s, 1H) 7.73 (br s, 1H). |
| 27 | 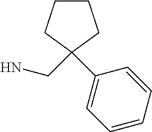 | 0.175 | 3-methoxy-4- (4-methyl-1H- imidazol-1-yl)- N-[(1-phenyl- cyclopentyl) methyl] benzamide | 390.2, LCMS | 1.74-1.82 (m, 2H), 1.89-1.96 (m, 4H), 2.02-2.08 (m, 2H), 2.28 (br s, 3H), 3.60 (d, J = 6.0 Hz, 2H), 3.87 (s, 3H), 5.83 (br t, J = 5.8 Hz, 1H), 6.92 (br s, 1H), 7.04 (dd, J = 8.1, 1.9 Hz, 1H), 7.22 (d, J = 8.1 Hz, 1H), 7.25-7.29 (m, 1H), 7.34-7.41 (m, 4H), 7.46 (d, J = 1.7 Hz, 1H), 7.70 (br s, 1H). |
| 28 | 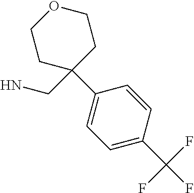 | 0.188 | 3-methoxy-4- (4-methyl-1H- imidazol-1-yl)- N-({4-[4-(tri- fluoromethyl) phenyl] tetrahydro- 2H-pyran-4- yl}methyl) benzamide | 474.2, LCMS | 2.03 (m, 2H), 2.16 (m, 2H), 2.28 (s, 3H), 3.63 (m, 2H), 3.75 (br d, J = 6 Hz, 2H), 3.88 (s, 3H), 3.92 (m, 2H), 5.88 (br t, J = 6.2 Hz, 1H), 6.93 (br s, 1H), 7.04 (br d, J = 8 Hz, 1H), 7.24 (d, J = 8.1 Hz, 1H), 7.45 (br s, 1H), 7.51 (d, J = 8.2 Hz, 2H), 7.70 (d, J = 8.2 Hz, 2H), 7.81 (br s, 1H). |
| 29 | 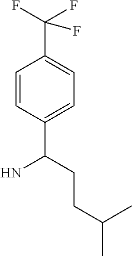 | 0.861 | 3-methoxy-4- (4-methyl-1H- imidazol-1-yl)- N-{4-methyl- 1-[4-(trifluoro- methyl)phenyl] pentyl} benzamide | 460.3, LCMS | 0.88 (d, J = 6.6 Hz, 6H), 1.15-1.36 (m, 2H), 1.59 (m, 1H), 1.85-2.00 (m, 2H), 2.26 (d, J = 1.0 Hz, 3H), 3.84 (s, 3H), 5.16 (m, 1H), 6.92 (m, 1H), 7.09 (br d, J = 7.8 Hz, 1H), 7.23 (d, J = 8.2 Hz, 1H), 7.35 (dd, J = 8.0, 1.8 Hz, 1H), 7.49 (d, J = 8.2 Hz, 2H), 7.59 (m, 3H), 7.66 (d, J = 1.4 Hz, 1H). |
| 30 | 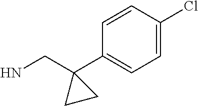 | 0.231 | N-{[1-(4- chlorophenyl) cyclopropyl] methyl}-3- methoxy-4-(4- methyl-1H- imidazol-1-yl) benzamide | 396.1, 398.1, LCMS | 0.91 (m, 2H), 1.01 (m, 2H), 2.27 (d, J = 1.0 Hz, 3H), 3.63 (d, J= 5.9 Hz, 2H), 3.89 (s, 3H), 6.33 (br t, J = 5.5 Hz, 1H), 6.93 (m, 1H), 7.17 (dd, J = 8.0, 1.8 Hz, 1H), 7.24 (d, J = 8.2 Hz, 1H), 7.28 (s, 4H), 7.51 (d, J = 1.8 Hz, 1H), 7.69 (d, J = 1.2 Hz, 1H). |
| 31 | 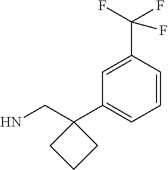 | 0.298 | 3-methoxy-4- (4-methyl-1H- imidazol-1-yl)- N-({1-[3-(tri- fluoromethyl) phenyl] cyclobutyl} methyl) benzamide | 444.2, LCMS | 1.92-2.00 (m, 1H), 2.28 (br s, 3H), 2.30-2.44 (m, 5H), 3.86 (d, J = 6.0 Hz, 2H), 3.89 (s, 3H), 5.95 (br t, J = 6.0 Hz, 1H), 6.93 (br s, 1H), 7.10 (dd, J = 8.2, 1.8 Hz, 1H), 7.24 (d, J = 8.1 Hz, 1H), 7.38 (m, 1H), 7.42 (br s, 1H), 7.49-7.52 (m, 3H), 7.71 (d, J = 1.2 Hz, 1H). |
| 32 | 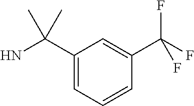 | 0.619 | 3-methoxy-4- (4-methyl-1H- imidazol-1-yl)- N-{1-methyl-1- [3-(trifluoro- methyl)phenyl] ethyl} benzamide | 418.1, LCMS | 1.84 (s, 6H), 2.30 (br s, 3H), 3.89 (s, 3H), 6.60 (br s, 1H), 6.96 (br s, 1H), 7.28-7.35 (m, 2H), 7.45-7.53 (m, 2H), 7.56 (d, J = 1.5 Hz, 1H), 7.63-7.68 (m, 2H), 7.74 (br s, 1H). |
| 33 | 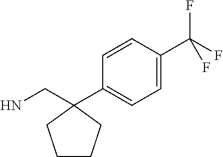 | 0.139 | 3-methoxy-4- (4-methyl- 1H-imidazol- 1-yl)-N-({1- [4-(trifluoro- methyl) phenyl] cyclopentyl} methyl) benzamide, formic acid salt | 458.2, LCMS | 1.79 (m, 2H), 1.93 (m, 4H), 2.07 (m, 2H), 2.32 (s, 3H), 3.64 (d, J = 6.3 Hz, 2H), 3.88 (s, 3H), 5.87 (br t, J = 5.8 Hz, 1H), 6.94 (s, 1H), 7.07 (br d, J = 8.0 Hz, 1H), 7.26 (m, 1H), 7.47 (m, 3H), 7.63 (br d J = 7.8 Hz, 2H), 8.08 (s, 1H), 8.31 (v br s, 1H) |
| 34 | 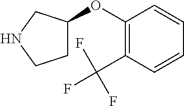 | 0.297 | 1-[2-methoxy- 4-({(3S)-3-[2- (trifluoro- methyl) phenoxy] pyrrolidin- 1-yl}carbonyl) phenyl]-4- methyl-1H- imidazole, formic acid salt | 446.3, LCMS | ¹H NMR (400 MHz, CD₃OD), mixture of 2 rotamers: d 2.27 and 2.28 (2 br s, 3H), 2.27-2.37 (m, 2H), 3.62- 3.90 (m, 4H), 3.87 and 3.94 (2 s, 3H), 5.22 and 5.34 (2 m, 1H), 7.05-7.13 (m, 1H), 7.17-7.33 (m, 4H), 7.45 and 7.51 (2 d, J = 8.0, 8.0 Hz, 1H), 7.53-7.63 (m, 2H), 8.06 and 8.10 (2 d, J = 1.2, 1.4 Hz, 1H), 8.17 (br s, 1H). |
| 35 | 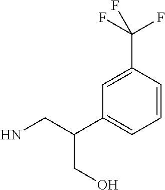 | 0.91 | N-{3-hydroxy- 2-[3-(trifluoro- methyl)phenyl] propyl}-3- methoxy-4-(4- methyl-1H- imidazol-1-yl) benzamide | 434.2, LCMS | ¹H NMR (400 MHz, CD₃OD) d 2.23 (d, J = 1.0 Hz, 3H), 3.32 (m, 1H, assumed, obscured by solvent peak), 3.70 (dd, half of ABX pattern, J = 13.6, 8.1 Hz, 1H), 3.79 (dd, half of ABX pattern J = 13.6, 6.9 Hz, 1H), 3.87 (d, J = 6.3, 2H), 3.90 (s, 3H), 7.10 (m, 1H), 7.38 (dd, half of AB quartet, J = 8.0, 1.6 Hz, 1H), 7.41 (d, half of AB quartet, J = 8.0 Hz, 1H), 7.50-7.64 (m, 5H), 7.84 (d, J = 1.4 Hz, 1H). |
| 36 | 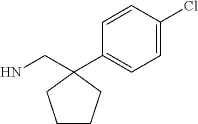 | 0.209 | N-{[1-(4- chlorophenyl) cyclopentyl] methyl}-3- methoxy-4- (4-methyl-1H- imidazol-1-yl) benzamide | 424.2, 426.2, LCMS | 1.73-1.79 (m, 2H), 1.84-1.93 (m, 4H), 1.98-2.03 (m, 2H), 2.27 (br s, 3H), 3.58 (br d, J = 8.0 Hz, 2H), 3.87 (s, 3H), 5.80 (m, 1H), 6.91 (br s, 1H), 7.03 (m, 1H), 7.21-7.28 (m, 3H), 7.31-7.34 (m, 2H), 7.46 (br s, 1H), 7.77 (br s, 1H). |
| 37 | 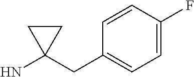 | 0.519 | N-[2-(4-fluoro- phenyl)-1,1- dimethylethyl]- 3-methoxy-4- (4-methyl-1H- imidazol-1-yl) benzamide | 382.6, LCMS | ¹H NMR (500 MHz, CD₃OD) 1.45 (s, 6H), 2.42 (d, J = 1.0 Hz, 3H), 3.20 (s, 2H), 3.97 (s, 3H), 6.97 (dd, J = 8.8, 8.8 Hz, 2H), 7.19 (dd, J = 8.8, 5.4 Hz, 2H), 7.46 (dd, J = 8.1, 1.8 Hz, 1H), 7.55 (m, 1H), 7.56-7.59 (m, 2H), 7.71 (br s, 1H), 9.03 (d, J = 1.5 Hz, 1H). |
| 38 | 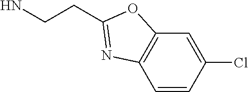 | 0.354 | N-[2-(6-chloro- 1,3-benzoxazol- 2-yl)ethyl]-3- methoxy-4-(4- methyl-1H- imidazol-1-yl) benzamide | 411.1, 413.1, LCMS | 2.28 (d, J = 1.0 Hz, 3H), 3.26 (br t, J = 6 Hz, 2H), 3.89 (s, 3H), 4.02 (dt, apparent q, J = 6, 6 Hz, 2H), 6.93 (m, 1H), 7.27 (d, J = 8.2 Hz, 1H), 7.31 (dd, J = 8.6, 2.0, 1H), 7.35 (dd, J = 8.1, 1.8 Hz, 1H), 7.48 (br t, J = 5.9 Hz, 1H), 7.51 (dd, J = 2.0, 0.4 Hz, 1H), 7.54 (dd, J = 8.5, 0.5 Hz, 1H), 7.59 (d, J = 1.8 Hz, 1H), 7.72 (d, J = 1.2 Hz, 1H). |
| 39 | 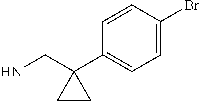 | 0.248 ^{†} | N-{[1-(4- bromophenyl) cyclopropyl] methyl}-3- methoxy-4-(4- methyl-1H- imidazol-1-yl) benzamide | 440.4, LCMS | ¹H NMR (500 MHz, CDCl₃) d 0.95 (m, 2H), 1.05 (m, 2H), 2.31 (s, 3H), 3.67 (d, J = 5.6 Hz, 2H), 3.92 (s, 3H), 6.41 (br t, J = 5.4 Hz, 1H), 6.97 (s, 1H), 7.21 (dd, J = 8.0, 1.7 Hz, 1H), 7.25-7.29 (m, 3H), 7.47 (d, J = 8.5 Hz, 2H), 7.55 (d, J = 1.5 Hz, 1H), 7.72 (br s, 1H) |
| 40 | 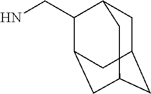 | 0.185 ^{†} | N-(adamantan- 2-ylmethyl)-3- methoxy-4-(4- methyl-1H- imidazol-1- yl)benzamide | 380.2, LCMS | 1.59 (m, 2H), 1.73- 1.98 (m, 13H), 2.29 (d, J = 1.0 Hz, 3H), 3.61 (dd, J = 7.6, 5.9 Hz, 2H), 3.91 (s, 3H), 6.36 (br t, J = 5.6 Hz, 1H), 6.94 (m, 1H), 7.27 (d, half of AB quartet, J = 8.0 Hz, 1H), 7.31 (dd, half of AB quartet, J = 8.1, 1.7 Hz, 1H), 7.61 (d, J = 1.6 Hz, 1H), 7.73 (d, J = 1.4 Hz, 1H). |
| 41 | 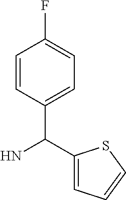 | 0.328 | N-[(4-fluoro- phenyl)(2- thienyl) methyl]-3- methoxy-4- (4-methyl-1H- imidazol-1- yl)benzamide, trifluoroacetic acid salt | 422.6, LCMS | ¹H NMR (500 MHz, CD₃OD) d 2.44 (d, J = 1.0 Hz, 3H), 3.99 (s, 3H), 6.67 (br s, 1H), 6.88 (m, 1H), 6.99 (dd, J = 5.1, 3.7 Hz, 1H), 7.13 (m, 2H), 7.38 (dd, J = 5.1, 1.2 Hz, 1H), 7.49 (dd, J = 8.5, 5.1 Hz, 2H), 7.62 (m, 1H), 7.64 (d, half of AB quartet, J = 8.3 Hz, 1H), 7.67 (dd, half of AB quartet, J = 8.2, 1.6 Hz, 1H), 7.78 (d, J = 1.7 Hz, 1H), 9.19 (d, J = 1.7 Hz, 1H). |
| 42 | 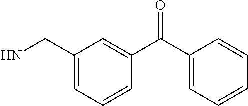 | 0.316 | N-(3-benzoyl- benzyl)-3- methoxy-4-(4- methyl-1H- imidazol-1-yl) benzamide | 426.3, LCMS | 2.28 (br s, 3H), 3.90 (s, 3H), 4.73 (d, J = 5.7 Hz, 2H), 6.94 (m, 2H), 7.27 (d, J = 8.2 Hz, 1H), 7.37 (dd, J = 8.2, 1.8 Hz, 1H), 7.45- 7.50 (m, 3H), 7.58- 7.63 (m, 3H), 7.68 (br d, J = 7.6 Hz, 1H), 7.73 (br s, 1H), 7.77-7.81 (m, 3H). |
| 43 | 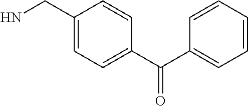 | 0.582 | N-(4-benzoyl- benzyl)-3- methoxy-4-(4- methyl-1H- imidazol-1-yl) benzamide | 426.2, LCMS | 2.28 (br s, 3H), 3.91 (s, 3H), 4.75 (d, J = 5.9 Hz, 2H), 6.95 (m, 1H), 7.08 (br t, J = 5.7 Hz, 1H), 7.26-7.29 (m, 1H), 7.41 (dd, J = 8.2, 1.8 Hz, 1H), 7.45-7.50 (m, 4H), 7.60 (m, 1H), 7.67 (m, 1H), 7.71 (br s, 1H), 7.76-7.79 (m, 4H). |
| 44 | 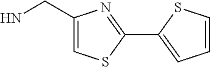 | 0.575 | 3-methoxy-4- (4-methyl-1H- imidazol-1-yl)- N-{[2-(2- thienyl)-1,3- thiazol-4-yl] methyl} benzamide | 411.3, LCMS | 2.28 (s, 3H), 3.86 (s, 3H), 4.74 (d, J = 5.5 Hz, 2H), 6.93 (br s, 1H), 7.07 (dd, J = 5.1, 3.7 Hz, 1H), 7.16 (s, 1H), 7.25 (d, J = 8.2 Hz, 1H), 7.37-7.42 (m, 2H), 7.45 (br t, J = 5.4 Hz, 1H), 7.49 (dd, J = 3.7, 0.8 Hz, 1H), 7.60 (d, J = 1.8 Hz, 1H), 7.73 (d, J = 1.2 Hz, 1H). |
| 45 | 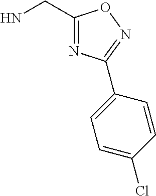 | 0.73 | N-{[3-(4- chlorophenyl)- 1,2,4-oxadiazol- 5-yl]methyl}-3- methoxy-4-(4- methyl-1H- imidazol-1-yl) benzamide, formic acid salt | 424.1, 426.1, LCMS | 2.28 (s, 3H), 3.91 (s, 3H), 4.96 (d, J = 5.7 Hz, 2H), 6.95 (m, 1H), 7.19 (br t, J = 5.3 Hz, 1H), 7.32 (d, J = 8.2 Hz, 1H), 7.41- 7.46 (m, 3H), 7.63 (d, J = 1.6 Hz, 1H), 7.75 (d, J = 1.2 Hz, 1H), 7.99 (d, J = 8.6 Hz, 2H). |
| 46 | 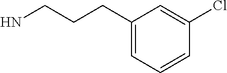 | 0.579 | N-[3-(3- chlorophenyl) propyl]-3- methoxy-4-(4- methyl-1H- imidazol-1-yl) benzamide | 383.7, 385.7, APCl | 2.00 (m, 2H), 2.31 (d, J = 1.0 Hz, 3H), 2.73 (t, J = 7.6 Hz, 2H), 3.53 (m, 2H), 3.93 (s, 3H), 6.11 (br m, 1H), 6.96 (m, 1H), 7.11 (br d, J = 7.5 Hz, 1H), 7.18-7.20 (m, 2H), 7.22-7.25 (m, 2H), 7.28 (d, J = 8.1 Hz, 1H), 7.57 (d, J = 1.8 Hz, 1H), 7.76 (d, J = 1.3 Hz, 1H). |
| 47 | 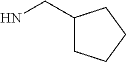 | 1.2 | N-cyclopentyl- methyl-3- methoxy-4-(4- methyl- imidazol-1-yl)- benzamide | 314 LCMS | 1.23-1.31 (m, 2H), 1.53-1.69 (comp, 4H), 1.76-1.86 (m, 2H), 2.20-2.12 (m 1H), 2.28 (s, 3H), 3.41 (dd, J = 7.2, 5.7 Hz, 2H), 3.91 (s, 3H), 6.18-6.24 (m, 1H), 6.93 (t, J = 1.2 Hz, 1H), 7.26 (s, 1H), 7.27 (s, 1H), 7.58 (s, 1H), 7.73 (d, J = 1.3 Hz, 1H). |
| 48 | 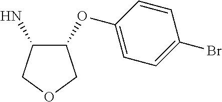 | 1.74 | N-[(3R,4R)- 4-(4- bromophenoxy)- tetrahydro-furan- 3-yl]-3- methoxy-4-(4- methylimidazol- 1-yl)benzamide | 472.0, LCMS | 2.30 (s, 3H), 3.79 (dd, J = 8.5, 7.8 Hz, 1H), 3.88 (s, 3H), 4.05 (dd, J = 10.7, 1.9 Hz, 1H), 4.20 (dd, J = 10.7, 4.4 Hz, 1H), 4.27 (dd, J = 8.5, 7.1 Hz, 1H), 4.92- 5.02 comp, 2H), 6.68 (d, J = 8.1 Hz, 1H), 6.80- 6.84 (comp, 2H), 6.93-6.95 (m, 1H), 7.17 (dd, J = 8.1, 1.7 Hz, 1H), 7.26-7.28 (comp, 2H), 7.39-7.42 (comp, 2H), 7.45 (d, J = 2.0 Hz, 1H), 7.75 (d, J = 1.5 Hz, 1H). |
| 123 | 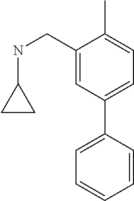 | 0.073 | N-cyclopropyl- 3-methoxy-N- [(4-methylbi- phenyl-3-yl) methyl]-4-(4- methyl-1H- imidazol-1-yl) benzamide | 452.1, LCMS, 2.83 min^{A} | |
| 124 | 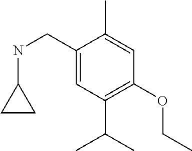 | 0.114 | N-cyclopropyl- N-(4-ethoxy-5- isopropyl-2- methylbenzyl)- 3-methoxy-4- (4-methyl-1H- imidazol-1- yl)benzamide | 462.2, LCMS | (evidence of rotamers) δ 0.50-0.61 (comp, 4 H), 1.19 (d, J = 6.8 Hz, 6H), 1.40 (t, J = 7.0 Hz, 3H), 2.27 (s, 3H), 2.28 (br s, 3H), 2.45 (br s, 3H) 3.29 (septet, J = 6.8 Hz, 1H), 3.81 (s, 3 H), 4.01 (q, J = 7.0 Hz, 2H), 4.67 (br s, 2H), 6.64 (s, 1H), 6.91 (s 1H), 7.08-7.16 (comp, 2H), 7.18- 7.23 (comp, 2H), 7.70 (s, 1H). |
| 125 | 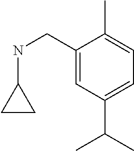 | 0.185 | N-cyclopropyl- N-(5-isopropyl- 2-methylbenzyl)- 3-methoxy-4- (4-methyl-1H- imidazol-1-yl) benzamide | 418.3, LCMS | (evidence of rotamers) δ 0.53- 0.66 (comp, 4H), 1.24 (d, J = 7.0 Hz, 6H), 2.29 (s, 3H), 2.29-2.36 (comp, 3H), 2.54 (br s, 1H), 2.89 (septet, J = 7.0 Hz, 1H), 3.83 (br s, 3H), 4.67-4.80 (comp, 2H), 6.92 (s, 1H), 7.06-7.10 (m, 1H), 7.10- 7.14 (m, 1H), 7.15-7.20 (comp, 2H), 7.23 (s, 1H), 7.80 (s, 1H), 8.28 (s, 1H). |
| 126 | 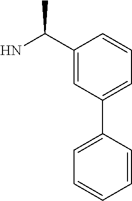 | 0.492 | N-[(1S)-1- biphenyl-3- ylethyl]-3- methoxy-4-(4- methyl-1H- imidazol-1-yl) benzamide | 412.2, LCMS, 2.77 min^{B} | |
| 127 | 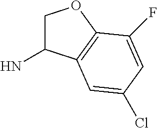 | 0.521 | N-(5-chloro-7- fluoro-2,3- dihydro-1- benzofuran-3- yl)-3-methoxy- 4-(4-methyl- 1H-imidazol- 1-yl)benzamide trifluoroacetate salt^{D} | 402.1, LCMS, 2.50 min^{A} | |
| 128 | 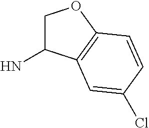 | 0.521 | N-(5-chloro- 2,3-dihydro-1- benzofuran-3- yl)-3-methoxy- 4-(4-methyl- 1H-imidazol-1- yl)benzamide^{E} | 384.1, LCMS | 2.27 (d, J = 1.0 Hz, 3H) 3.92 (s, 3H) 4.47 (dd, J = 10.3, 3.9 Hz, 1H), 4.81 (dd, J = 10.3, 8.0 Hz, 1H) 5.77 (td, J = 7.8, 3.9 Hz, 1H) 6.47 (d, J = 7.8 Hz, 1H) 6.81 (d, J = 8.8 Hz, 1H) 6.93, (s, 1H) 7.21 (d, J = 7.3 Hz, 1H), 7.23 (d, J = 8.6 Hz, 1H), 7.27 (d, J = 1.0 Hz, 1H) 7.35 (d, J = 2.3 Hz, 1H) 7.57 (s, 1H) 7.75 (d, J = 1.2 Hz, 1H) |
| 129 | 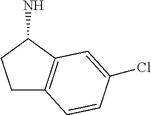 | 0.605 | N-[(1S)-6- chloro-2,3- dihydro-1H- inden-1-yl]-3- methoxy-4-(4- methyl-1H- imidazol-1- yl)benzamide | 382.1, LCMS, 2.42 min^{A} | |
| 130 | 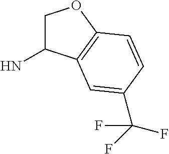 | 0.666 | 3-methoxy-4- (4-methyl-1H- imidazol-1-yl)- N-[5-(trifluoro- methyl)-2,3- dihydro-1- benzofuran-3- yl]benzamide^{D} | 382.1, LCMS, 2.42 min^{A} | |
| 131 | 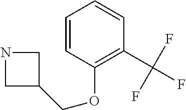 | 0.311 | 1-{2-methoxy- 4-[(3-{[2- (trifluoro- methyl) phenoxy] methyl} azetidin-1- yl)carbonyl] phenyl}-4- methyl-1H- imidazole | 446.0, LCMS, 2.58 min^{A} | |
| 132 | 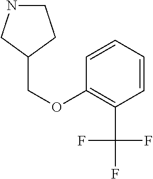 | 0.472 | 1-{2-methoxy- 4-[(3-{[2-(tri- fluoromethyl) phenoxy] methyl} pyrrolidin-1- yl)carbonyl] phenyl}-4- methyl-1H- imidazole | 460.1, LCMS, 2.53 min^{C} | |
| 133 | 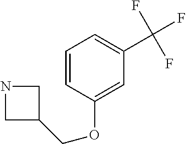 | 0.496 | 1-{2-methoxy- 4-[(3-{[3- (trifluoromethyl) phenoxy]methyl} azetidin-1-yl) carbonyl] phenyl}-4- methyl-1H- imidazole | 446.0, LCMS, 2.56 min^{A} | |
| 134 | 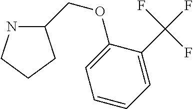 | 0.675 | 1-{2-methoxy- 4-[(2-{[2-(tri- fluoromethyl) phenoxy] methyl} pyrrolidin-1- yl)carbonyl] phenyl}-4- methyl-1H- imidazole | 460.0, LCMS, 2.94 min^{C} | |

^{*}Geometric mean or 2-8 determinations
^{**}Geometric mean of 26 determinations
^{†}Single IC₅₀ determination
^{A}QC conditions: Column: Waters Atlantis dC₁₈ 4.6 × 50, 5 μm; Mobile phase A: 0.05% TFA in water (v/v); Mobile phase B: 0.05% TFA in acetonitrile (v/v); Gradient: 95% H₂O/5% MeCN linear to 5% H₂O/95% MeCN in 4.0 min, HOLD at 5% H₂O/95% MeCN to 5.0 min. Flow: 2.0 mL/min.
^{B}QC conditions: Column: Waters XBridge C₁₈ 4.6 × 50, 5 μm; Mobile phase A: 0.03% NH₄OH in water (v/v); Mobile phase B: 0.03% NH₄OH in acetonitrile (v/v); Gradient: 85% H₂O/15% MeCN linear to 5% H₂O/95% MeCN in 4.0 min, HOLD at 5% H₂O/95% MeCN to 5.0 min. Flow: 2.0 mL/min.
^{C}QC conditions: Column: Waters XBridge C₁₈ 4.6 × 50, 5 μm; Mobile phase A: 0.03% NH₄OH in water (v/v); Mobile phase B: 0.03% NH₄OH in acetonitrile (v/v); Gradient: 90% H₂O/10% MeCN linear to 5% H₂O/95% MeCN in 4.0 min, HOLD at 5% H₂O/95% MeCN to 5.0 min. Flow: 2.0 mL/min.
^{D}Single enantiomer, absolute stereochemistry unknown. The enantiomers of the final compound were separated using chiral prep-HPLC.
^{E}Single enantiomer, absolute stereochemistry unknown. This compound was prepared using 5-chloro-2,3-dihydro-1-benzofuran-3-amine enantiomer 2 from the experimental section preceding this table.

### Library Format Syntheses

Preparation of N-substituted 3-methoxy-4(4-methyl-1H-imidazol-1-yl)benzamides:

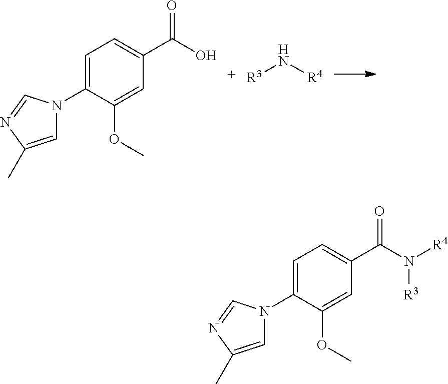

To a vial charged with the an amine (0.075 mmol) was added a solution of 3-methoxy-4-(4-methyl-1H-imidazol-1-yl)benzoic acid (17.41 mg, 0.075 mmol) in dimethylformamide (0.6 mL). Next, diisopropylethylamine (0.025 ml, 0.255 mmol) and O-(1H-benzotriazol-1-yl)-1,1,3,3-tetramethyluronium hexafluorophosphate (HBTU, 97%, 40 mg, 0.9 mmol) in dimethylformamide (0.3 mL) was added, and the reaction was shaken at room temperature for 16 hours. The solvents were removed in vacuo, and the residue was redissolved in dichloroethane (2 mL). The mixture was vortexed for 7 minutes, and saturated sodium bicarbonate (2 mL) was added. The mixture was shaken for 5 minutes, whereupon the layers were separated and the aqueous layer was extracted with dichloroethane (2 mL). The combined organic layers were concentrated in vacuo, and the residue was dissolved into dimethyl sulfoxide (1 mL) and purified by preparative HPLC by one of the following preparative HPLC methods: Method 1 (column: XBridge C18, 5 um, 19×100 mm; Solvent A: 0.1% ammonium hydroxide in water (v/v); Solvent B: 0.1% ammonium hydroxide in acetonitrile (v/v) using the appropriate gradients). Method 2(column: XBridge C18, 5 um, 19×50 mm; Solvent A: 0.1% ammonium hydroxide in water (v/v); Solvent B: 0.1% ammonium hydroxide in acetonitrile (v/v) using the appropriate gradients). Method 3 (column: XTerra MS C18, 5 um, 19×50 mm Solvent A: 0.1% trifluoroacetic acid in water (v/v); Solvent B: 0.1% trifluoroacetic acid in acetonitrile (v/v) using the appropriate gradients). Method 4 (column: Sunfire C18, 5 um, 19×100 mm Solvent A: 0.1% trifluoroacetic acid in water (v/v); Solvent B: 0.1% trifluoroacetic acid in acetonitrile (v/v) using the appropriate gradients). Method 5 (column: Symmetry C18, 5 um, 30×50 mm Solvent A: 0.05% trifluoroacetic acid in water (v/v); Solvent B: 0.05% trifluoroacetic acid in acetonitrile (v/v) using the appropriate gradients). Note: in some cases, this general procedure was slightly modified: instead of using dimethylformamide as the solvent and adding diisopropylamine neat, a (0.5M) solution of diisopropylamine in dimethylformamide (or dimethylacetamide) was used to dissolve the starting amine (0.3 mL) and 3-methoxy-4-(4-methyl-1H-imidazol-1-yl)benzoic acid (0.3 mL).

TABLE 3

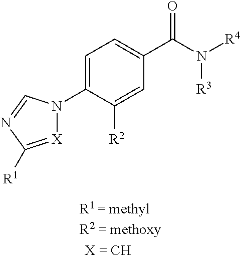

| | | | Aβ 42 | Mass Spec: | Ret. |
|---|---|---|---|---|---|
| | | | IC₅₀ | Observed | Time |
| Ex# | IUPAC Name | Structure of NR³R⁴ | (nM)^{†} | ion (M + 1) | (min.) |
| 49 | N-{[5-(4-chlorophenyl)- 2-thienyl]methyl}-3- methoxy-4-(4-methyl- 1H-imidazol-1-yl) benzamide | 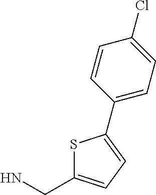 | 137 nM | 438.21 | 4.27^{A} |
| 50 | 3-methoxy-4-(4-methyl- 1H-imidazol-1-yl)-N- ({2-[4-(trifluoromethyl) phenyl]-1,3-thiazol-4- yl}methyl)benzamide | 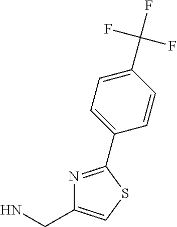 | 152 nM | 473.27 | 4.05^{A} |
| 51 | N-[2-(5,7-dichloro-2- methyl-1H-indol-3-yl) ethyl]-3-methoxy-4-(4- methyl-1H-imidazol- 1-yl)benzamide | 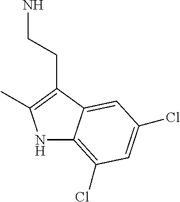 | 189 nM | 457.25 | 4.04^{A} |
| 52 | N-[2-(5-bromo-1H- indol-3-yl)ethyl]-3- methoxy-4-(4-methyl- 1H-imidazol-1-yl) benzamide | 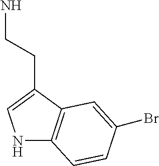 | 251 nM | 453.24 | 3.76^{A} |
| 53 | N-{[2-(4-chlorophenyl)- 1,3-thiazol-4-yl]methyl}- 3-methoxy-4-(4-methyl- 1H-imidazol-1-yl) benzamide | 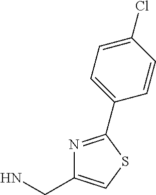 | 306 nM | 439.22 | 3.92^{A} |
| 54 | N-(3-chloro-4- fluorobenzyl)-3-methoxy- 4-(4-methyl-1H-imidazol- 1-yl)benzamide | 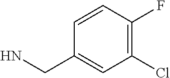 | 326 nM | 374.00 | 1.27^{B} |
| 55 | 3-methoxy-4-(4-methyl- 1H-imidazol-1-yl)-N- [3-(trifluoromethoxy) benzyl]benzamide | 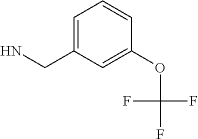 | 332 nM | 406.01 | 1.35^{B} |
| 56 | N-[2-(2,6-dimethyl- phenoxy)-1-methylethyl]- 3-methoxy-4- (4-methyl-1H- imidazol-1-yl) benzamide | 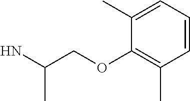 | 337 nM | 394.44 | 1.33^{B} |
| 57 | 3-methoxy-4-(4- methyl-1H-imidazol- 1-yl)-N-[4-(trifluoro- methoxy)benzyl] benzamide | 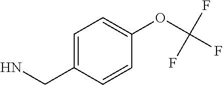 | 339 nM | 406.06 | 1.37^{B} |
| 58 | N-[(3-chloro-4,7- difluoro-1-benzothien- 2-yl)methyl]-3- methoxy-4-(4-methyl- 1H-imidazol-1-yl) benzamide | 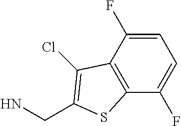 | 346 nM | 448.20 | 4.12^{A} |
| 59 | N-(4-chloro-2- methylbenzyl)-3- methoxy-4-(4-methyl- 1H-imidazol-1-yl) benzamide | 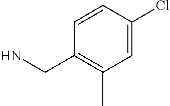 | 351 nM | 370.03 | 1.33^{B} |
| 60 | 3-methoxy-4-(4- methyl-1H-imidazol- 1-yl)-N-(3- phenoxybenzyl) benzamide | 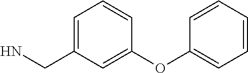 | 353 nM | 414.22 | 3.43^{C} |
| 61 | 3-methoxy-4-(4- methyl-1H-imidazol- 1-yl)-N-(4-phenyl- butyl)benzamide | 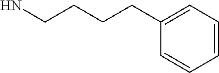 | 356 nM | 364.41 | 1.32^{B} |
| 62 | N-[3-fluoro-5- (trifluoromethyl) benzyl]-3-methoxy- 4-(4-methyl-1H- imidazol-1-yl) benzamide | 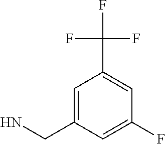 | 369 nM | 408.06 | 1.37^{B} |
| 63 | 3-methoxy-N- [(1S,2S)-2-(3- methoxybenzyl) cyclopentyl]-4- (4-methyl-1H- imidazol-1-yl) benzamide | 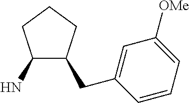 | 391 nM | 420.48 | 1.36^{B} |
| 64 | N-[2-(3,4-dimethyl- phenyl)ethyl]-3- methoxy-4-(4- methyl-1H-imidazol- 1-yl)benzamide | 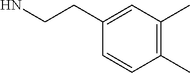 | 395 nM | 364.40 | 1.31^{B} |
| 65 | 3-methoxy-N-{[1- (3-methoxyphenyl) cyclopentyl]methyl}- 4-(4-methyl-1H- imidazol-1-yl) benzamide | 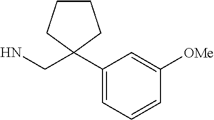 | 410 nM | 420.25 | 3.42^{C} |
| 66 | N-[4-fluoro-3- (trifluoromethyl) benzyl]-3-methoxy- 4-(4-methyl-1H- imidazol-1-yl) benzamide | 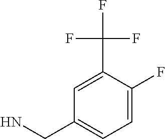 | 410 nM | 408.02 | 1.35^{B} |
| 67 | N-(3,4-dichlorobenzyl)- 3-methoxy- 4-(4-methyl-1H- imidazol-1-yl) benzamide | 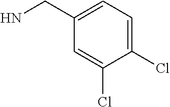 | 410 nM | 390.12 | 3.27^{C} |
| 68 | N-[2-(7-chloro-1,3- benzoxazol-2-yl) ethyl]-3-methoxy- 4-(4-methyl-1H- imidazol-1-yl) benzamide | 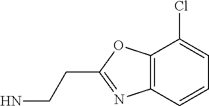 | 418 nM | 411.23 | 3.45^{A} |
| 69 | 1-[2-methoxy-4- ({(3R)-3-[2- (trifluoromethyl) phenoxy]pyrrolidin- 1-yl}carbonyl) phenyl]-4-methyl- 1H-imidazole | 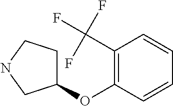 | 421 nM^{*} | 446.09 | 1.32^{B} |
| 70 | N-[(3-chloro-1- benzothien-2-yl) methyl]-3-methoxy- 4-(4-methyl-1H- imidazol-1-yl) benzamide | 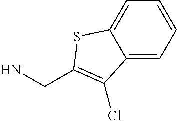 | 425 nM | 412.20 | 3.90^{A} |
| 71 | N-[2-(2,5- dimethylphenyl)ethyl]- 3-methoxy-4-(4- methyl-1H- imidazol-1-yl) benzamide | 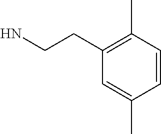 | 429 nM | 364.40 | 1.32^{B} |
| 72 | N-[(1R,2R)-2- benzylcyclopentyl]- 3-methoxy-4-(4- methyl-1H- imidazol-1-yl) benzamide | 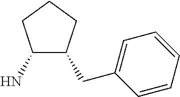 | 442 nM | 390.44 | 1.39^{B} |
| 73 | N-[2-(2,5-dimethyl- 1H-indol-3-yl)ethyl]- 3-methoxy-4-(4- methyl-1H-imidazol- 1-yl)benzamide | 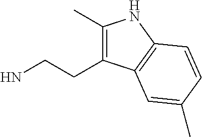 | 444 nM | 403.32 | 3.60^{A} |
| 74 | N-[2-(2- chlorophenoxy)propyl]- 3-methoxy-4-(4- methyl-1H-imidazol- 1-yl)benzamide | 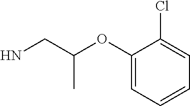 | 466 nM | 400.38 | 1.28^{B} |
| 75 | 3-methoxy-4-(4- methyl-1H-imidazol- 1-yl)-N-[2- (trifluoromethoxy)benzyl] benzamide | 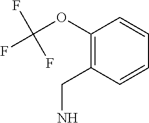 | 469 nM | 406.01 | 1.33^{B} |
| 76 | N-(2-chloro-4- fluorobenzyl)-3-methoxy- 4-(4-methyl-1H-imidazol- 1-yl)benzamide | 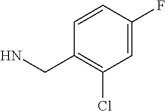 | 476 nM | 374.04 | 1.26^{B} |
| 77 | N-[2-fluoro-3- (trifluoromethyl)benzyl]- 3-methoxy-4-(4-methyl- 1H-imidazol-1-yl) benzamide | 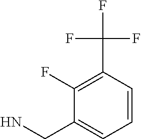 | 481 nM | 408.05 | 1.35^{B} |
| 78 | N-[1-(4-chlorophenyl) cyclopropyl]-3- methoxy-4-(4-methyl- 1H-imidazol-1-yl) benzamide | 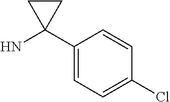 | 488 nM | 382.24 | 3.60^{A} |
| 79 | N-[2-(2-chlorophenyl) ethyl]-3-methoxy-4-(4- methyl-1H-imidazol- 1-yl)benzamide | 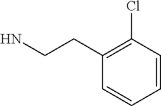 | 495 nM | 370.34 | 1.24^{B} |
| 80 | N-[(1R,2S)-2-hydroxy- 1,2-diphenylethyl]-3- methoxy-4-(4-methyl- 1H-imidazol-1-yl) benzamide | 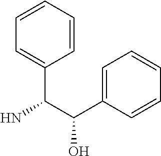 | 501 nM | 428.06 | 1.18^{B} |
| 81 | N-[3-(4-fluorophenyl) propyl]-3-methoxy-4- (4-methyl-1H-imidazol- 1-yl)benzamide | 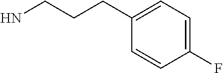 | 504 nM | 368.39 | 1.26^{B} |
| 82 | N-(2,5-dimethylbenzyl)- 3-methoxy-4-(4-methyl- 1H-imidazol-1-yl) benzamide | 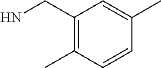 | 513 nM | 350.22 | 3.09^{C} |
| 83 | N-[3-fluoro-4- (trifluoromethyl)benzyl]- 3-methoxy-4-(4-methyl- 1H-imidazol-1-yl) benzamide | 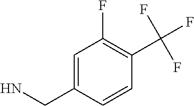 | 514 nM | 408.06 | 1.37^{B} |
| 84 | N-(3-chlorobenzyl)-3- methoxy-4-(4-methyl- 1H-imidazol-1-yl) benzamide | 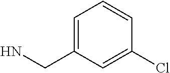 | 520 nM | 356.06 | 1.25^{B} |
| 85 | 3-(2-isopropylphenoxy)- 1-[3-methoxy-4-(4- methyl-1H-imidazol- 1-yl)benzoyl]piperidine | 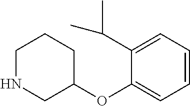 | 533 nM | 434.51 | 1.45^{B} |
| 86 | N-[(5-chloro-6-methyl- 1,3-benzoxazol-2-yl) methyl]-3-methoxy-4- (4-methyl-1H-imidazol- 1-yl)benzamide | 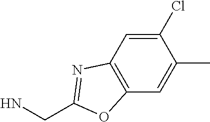 | 544 nM | 411.23 | 3.69^{A} |
| 87 | N-(1-benzothien-2- ylmethyl)-3-methoxy- 4-(4-methyl-1H- imidazol-1-yl) benzamide | 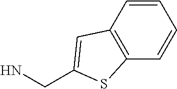 | 560 nM | 378.24 | 3.70^{A} |
| 88 | 3-methoxy-4-(4- methyl-1H-imidazol- 1-yl)-N-{1-methyl-2- [4-(trifluoromethyl) phenyl]ethyl} benzamide | 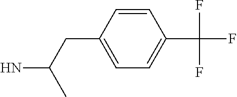 | 561 nM | 418.43 | 1.37^{B} |
| 89 | N-[(5-fluoro-1H- indol-2-yl)methyl]- 3-methoxy-4-(4- methyl-1H-imidazol- 1-yl)benzamide | 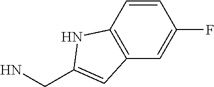 | 570 nM | 379.25 | 3.60^{A} |
| 90 | N-[2-(2- chlorophenoxy)ethyl]-3- methoxy-4-(4-methyl- 1H-imidazol-1-yl) benzamide | 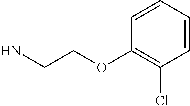 | 573 nM | 386.36 | 1.20^{B} |
| 91 | 1-[3-methoxy-4-(4- methyl-1H-imidazol- 1-yl)benzoyl]-4-[2- (trifluoromethyl) phenoxy]piperidine | 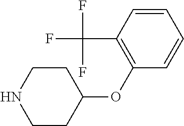 | 588 nM | 460.47 | 1.42^{B} |
| 92 | N-[1-(3-chlorobenzyl) cyclopropyl]-3- methoxy-4-(4-methyl- 1H-imidazol-1-yl) benzamide | 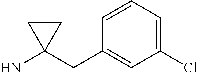 | 595 nM | 396.19 | 3.21^{C} |
| 93 | 1-[3-methoxy-4-(4- methyl-1H-imidazol- 1-yl)benzoyl]-4-[2- (trifluoromethoxy) phenoxy]piperidine | 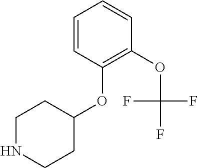 | 602 nM | 476.51 | 1.44^{B} |
| 94 | N-({3-[3-fluoro-4- (trifluoromethyl) phenyl]-1,2,4- oxadiazol-5-yl} methyl)-3-methoxy- 4-(4-methyl-1H- imidazol-1-yl) benzamide | 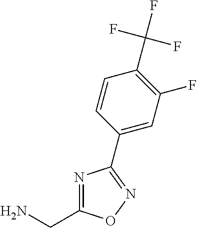 | 610 nM | 476.28 | 3.96^{A} |
| 95 | N-[(1R,2R)-2-(3- fluorobenzyl) cyclopentyl]-3- methoxy-4-(4- methyl-1H- imidazol-1-yl) benzamide | 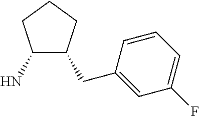 | 627 nM | 408.47 | 1.39^{B} |
| 96 | N-[(2-chlorophenyl)(1- methyl-1H- benzimidazol-2-yl) methyl]-3-methoxy- 4-(4-methyl-1H- imidazol-1-yl) benzamide | 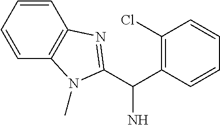 | 630 nM | 486.31 | 3.68^{A} |
| 97 | 3-methoxy-4-(4- methyl-1H-imidazol- 1-yl)-N-[2-(5-methyl- 1H-indol-3-yl)ethyl] benzamide | 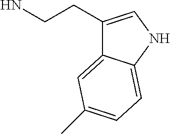 | 634 nM | 389.31 | 3.51^{A} |
| 98 | 1-(4-{[3-(3- chlorophenyl)pyrrolidin- 1-yl]carbonyl}-2- methoxyphenyl)-4- methyl-1H-imidazole | 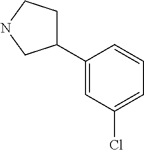 | 642 nM | 396.41 | 1.30^{B} |
| 99 | N-[1-(1H-indol-5- yl)ethyl]-3-methoxy- 4-(4-methyl-1H- imidazol-1-yl) benzamide | 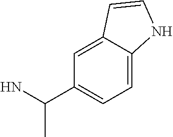 | 665 nM | 375.11 | 1.17^{B} |
| 100 | 3-methoxy-4-(4- methyl-1H-imidazol- 1-yl)-N-(1-methyl- 3-phenylpropyl) benzamide | 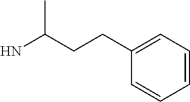 | 667 nM | 364.40 | 1.29^{B} |
| 101 | 3-methoxy-4-(4-methyl- 1H-imidazol-1-yl)-N- [(1-phenylcyclopropyl) methyl]benzamide | 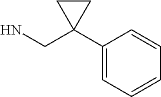 | 686 nM | 362.20 | 3.06^{C} |
| 102 | 3-methoxy-4-(4-methyl- 1H-imidazol-1-yl)-N- {2-methyl-6-[(methyl- sulfonyl)amino]benzyl} benzamide | 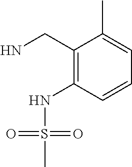 | 689 nM | 429.05 | 1.15^{B} |
| 103 | N-[2-(4-chlorophenyl) ethyl]-3-methoxy-4-(4- methyl-1H-imidazol-1- yl)benzamide | 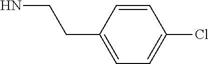 | 698 nM | 370.18 | 3.11^{C} |
| 104 | N-[2-(2-ethoxyphenyl) ethyl]-3-methoxy-4-(4- methyl-1H-imidazol- 1-yl)benzamide | 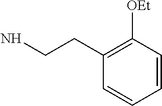 | 698 nM | 380.41 | 1.27^{B} |
| 105 | 3-methoxy-N-{[1-(4- methoxyphenyl) cyclopentyl]methyl}- 4-(4-methyl-1H- imidazol-1-yl) benzamide | 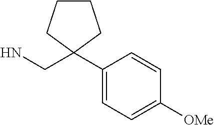 | 708 nM | 420.25 | 3.41^{C} |
| 106 | N-(5-hydroxy-1,2,3,4- tetrahydronaphthalen- 1-yl)-3-methoxy-4-(4- methyl-1H-imidazol-1- yl)benzamide | 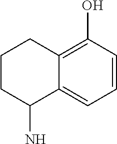 | 731 nM | 378.08 | 1.09^{B} |
| 107 | N-[(4-fluorophenyl)(1- methyl-1H- benzimidazol-2-yl) methyl]-3-methoxy-4- (4-methyl-1H-imidazol- 1-yl)benzamide | 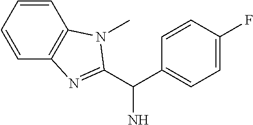 | 735 nM | 470.34 | 3.87^{A} |
| 108 | 1-(4-{[3-(2,5- dichlorophenoxy)azetidin- 1-yl]carbonyl}-2- methoxyphenyl)-4- methyl-1H-imidazole | 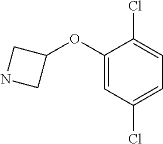 | 738 nM^{*} | 432.02 | 1.39^{B} |
| 109 | 1-(4-{[3-(2,5- dimethoxyphenyl) pyrrolidin-1-yl] carbonyl}-2- methoxyphenyl)- 4-methyl-1H- imidazole | 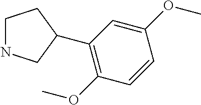 | 739 nM | 422.32 | 3.62^{A} |
| 110 | 3-methoxy-4- (4-methyl-1H- imidazol-1-yl)-N- [(1S)-1-(4- methylphenyl)ethyl) benzamide | 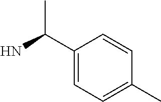 | 747 nM | 350.09 | 1.27^{B} |
| 111 | 3-methoxy-4-(4-methyl- 1H-imidazol-1-yl)-N- {2-morpholin-4-yl-2-[4- (trifluoromethyl)phenyl] ethyl}benzamide | 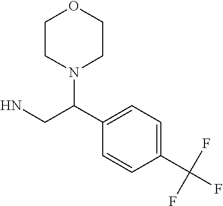 | 756 nM | 489.25 | 2.46^{C} |
| 112 | N-[2-(3-ethoxyphenyl) ethyl]-3-methoxy-4-(4- methyl-1H-imidazol-1- yl)benzamide | 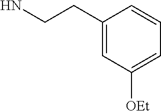 | 765 nM | 380.41 | 1.23^{B} |
| 113 | 3-methoxy-4-(4-methyl- 1H-imidazol-1-yl)-N- (1,2,3,4-tetrahydro- naphthalen-2-ylmethyl) benzamide | 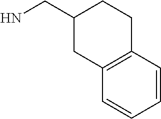 | 784 nM | 376.42 | 1.33^{B} |
| 114 | 3-methoxy-4-(4-methyl- 1H-imidazol-1-yl)-N- (1,2,3,4-tetrahydro- naphthalen-2-yl) benzamide | 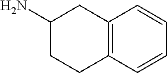 | 793 nM | 362.38 | 1.26^{B} |
| 115 | N-[(1R,2R)-2-(4- fluorophenyl) cyclopentyl]-3- methoxy-4-(4- methyl-1H-imidazol- 1-yl)benzamide | 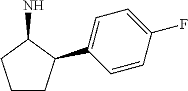 | 798 nM | 394.24 | 3.18^{C} |
| 116 | N-{[5-(4- fluorophenyl)isoxazol- 3-yl]methyl}-3- methoxy-4-(4- methyl-1H-imidazol- 1-yl)benzamide | 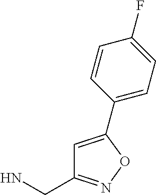 | 815 nM | 407.27 | 3.54^{A} |
| 117 | 3-methoxy-4-(4- methyl-1H-imidazol- 1-yl)-N-[2-(2-methyl- 1H-indol-3-yl)ethyl] benzamide | 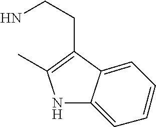 | 828 nM | 389.31 | 3.46^{A} |
| 118 | 3-methoxy-N-methyl- 4-(4-methyl-1H- imidazol-1-yl)-N-(1- naphthylmethyl) benzamide | 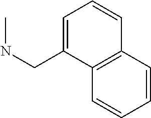 | 837 nM | 386.23 | 3.17^{C} |
| 119 | 1-[3-methoxy-4-(4- methyl-1H-imidazol- 1-yl)benzoyl]-4-[3- (trifluoromethyl) phenoxy]piperidine | 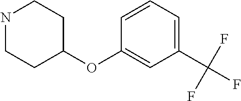 | 847 nM | 460.47 | 1.45^{B} |
| 120 | 3-methoxy-N-(5- methoxy-2,3-dihydro- 1H-inden-1-yl)-4-(4- methyl-1H-imidazol- 1-yl)benzamide | 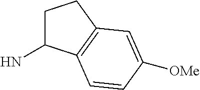 | 878 nM^{*} | 378.41 | 1.20^{B} |
| 121 | 3-methoxy-N-[2-(5- methyl-1,3-benzoxazol- 2-yl)ethyl]-4-(4-methyl- 1H-imidazol-1-yl) benzamide | 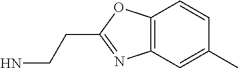 | 880 nM | 391.29 | 3.44^{A} |
| 122 | N-(3,4-dihydro-2H- chromen-3-ylmethyl)- 3-methoxy-4-(4- methyl-1H-imidazol- 1-yl)benzamide | 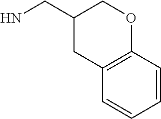 | 976 nM | 378.41 | 1.19^{B} |

^{†}Geometric mean of 2-8 determinations
^{*}Single IC₅₀ determination
^{A}Column: Waters Xterra C₁₈ 3.5 μm, 4.6 × 50 mm; Mobile phase A: 0.1% NH₄OH in water; Mobile phase B: 0.1% NH₄OH in CH₃CN;
Flow rate 2.75 mL/min

| | |
|---|---|
| Gradient: | |
| 0 minutes | 5% B |
| 5.83 minutes | 95% B |
| 9.0 minutes | 95% B |

^{B}Column: Advanced Materials Technology Halo C₁₈ 2.7 μm, 3.0 × 30 mm; Mobile phase A: 0.01% TFA in water; Mobile phase B: 0.01%
TFA in CH₃CN; Flow rate 1.5 mL/min

| | |
|---|---|
| Gradient: | |
| 0 minutes | 5% B |
| 2.30 minutes | 95% B |
| 2.50 minutes | 95% B |

^{C}Column: Waters Sunfire C₁₈ 3.5 μm, 4.6 × 50 mm; Mobile phase A: 0.1% TFA in water; Mobile phase B: 0.01% TFA in CH₃CN; Flow
rate 2.75 mL/min

| | |
|---|---|
| Gradient: | |
| 0 minutes | 5% B |
| 5.83 minutes | 95% B |
| 9.0 minutes | 95% B |

Preparation of N-substituted 3-methoxy-4(4-methyl-1H-imidazol-1-yl)benzamides:

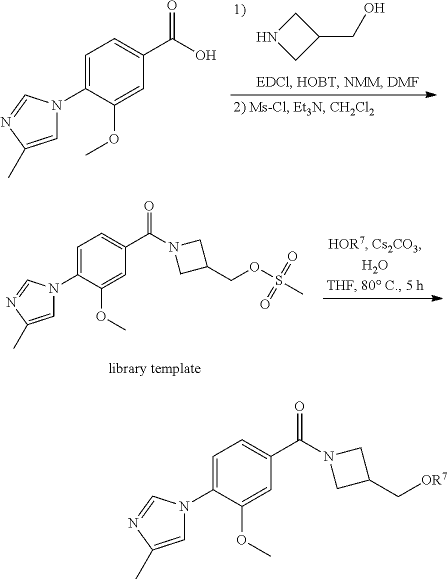

The requisite library template {1-[3-methoxy-4-(4-methyl-1H-imidazol-1-yl)benzoyl]azetidin-3-yl}methyl methanesulfonate was prepared in two steps from 3-methoxy-4-(4-methyl-1H-imidazol-1-yl)benzoic acid [Example 1] using methods well know to those skilled in the art.

To a vial charged with the phenol (0.075 mmol) was added a solution of {1-[3-methoxy-4-(4-methyl-1H-imidazol-1-yl)benzoyl]azetidin-3-yl}methyl methanesulfonate (19.0 mg, 0.050 mmol) in THF (0.5 mL). Next, Cs₂CO₃ (32.6 mg, 0.100 mmol) and water (1 drop) was added to each vial, and the reactions were shaken at 80° C. for 5 hours. The solvents were removed in vacuo, and the residue was purified by prep-HPLC.

TABLE 4

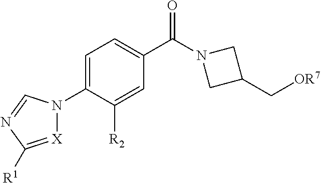

| | | | Aβ 42 | Mass Spec: | Ret. |
|---|---|---|---|---|---|
| | | | IC₅₀ | Observed ion | Time |
| Ex# | IUPAC Name | Structure of OR⁷ | (nM)^{†} | (M + 1) | (min.) |
| 135 | 1-[2-methoxy-4- ({3-[(1-naphthyloxy) methyl]azetidin-1- yl}carbonyl)phenyl]- 4-methyl-1H- imidazole trifluoroacetate salt | 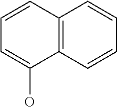 | 111 nM | 428 | 2.55^{A} |
| 136 | 1-[4-({3-[(2-chloro- 4-fluoro-3-methyl- phenoxy)methyl] azetidin-1-yl} carbonyl)-2- methoxyphenyl]- 4-methyl-1H- imidazole trifluoroacetate salt | 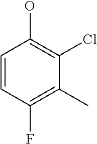 | 311 nM | 444 | 2.547^{A} |
| 137 | 1-[4-({3-[(2,3- dihydro-1H-inden- 4-yloxy)methyl] azetidin-1-yl} carbonyl)-2- methoxyphenyl]- 4-methyl-1H- imidazole trifluoroacetate salt | 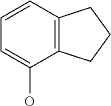 | 411 nM | 418 | 2.57^{A} |
| 138 | 1-[4-({3-[(2-ethyl- 4-fluorophenoxy) methyl]azetidin-1- yl}carbonyl)-2- methoxyphenyl]- 4-methyl-1H- imidazole trifluoroacetate salt | 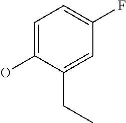 | 537 nM | 424 | 2.564^{A} |
| 139 | 1-[4-({3-[(chloro- 5-methylphenoxy) methyl]azetidin- 1-yl}carbonyl)-2- methoxyphenyl]- 4-methyl-1H- imidazole trifluoroacetate salt | 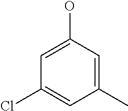 | 557 nM | 426 | 2.622^{A} |
| 140 | 1-[4-({3-[(2- ethylphenoxy) methyl]azetidin- 1-yl}carbonyl)-2- methoxyphenyl]- 4-methyl-1H- imidazole trifluoroacetate salt | 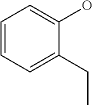 | 593 nM | 406 | 2.76^{B} |

^{†}Geometric mean of at least 2 determinations.
^{A}Column: Welch XB-C18 2.1 × 50 mm, 5 μm; Mobile Phase A: 0.0375% TFA in water;
Mobile Phase B: 0.01875% TFA in acetonitrile; Flow rate 0.8 mL/min

| | |
|---|---|
| Gradient: | |
| 0 minutes | 10% B |
| 0.5 minutes | 10% B |
| 4.0 minutes | 100% B |

^{B}Column: Welch XB-C18 2.1 × 50 mm, 5 μm; Mobile Phase A: 0.0375% TFA in water;
Mobile Phase B: 0.01875% TFA in acetonitrile; Flow rate 0.8 mL/min

| | |
|---|---|
| Gradient: | |
| 0 minutes | 1% B |
| 0.6 minutes | 5% B |
| 4.0 minutes | 100% B |

1. A compound of formula Ia: 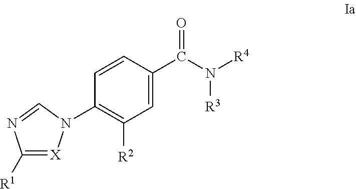
wherein
X is CH or N;
R¹ is hydrogen, C₁₋₆alkyl, C₃₋₆cycloalkyl, or C₂₋₆alkenyl, wherein said alkyl, cycloalkyl and alkenyl is optionally substituted with one to three halogen or —(CH₂)ₜ—C₃₋₆cycloalkyl;
R² is hydrogen, —CF₃, cyano, halogen, C₁₋₆alkyl, or —OR⁵;
R³ and R⁴ are each independently hydrogen, C₁₋₆alkyl, C₂₋₆alkenyl, —(CH₂)ₜ-cycloalkyl, —(CH₂)ₜ-heterocycloalkyl, —(CH₂)ₜ-aryl, or —(CH₂)ₜ-heteroaryl; wherein said alkyl, alkenyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl R³ or R⁴ substituent is optionally independently substituted with one to three R⁶; or R³ and R⁴ together with the nitrogen they are bonded to form a heterocycloalkyl moiety, wherein said heterocycloalkyl moiety is optionally independently substituted with one to three R⁶;
R⁵ is hydrogen, C₁₋₆alkyl, C₃₋₆cycloalkyl, C₂₋₆alkenyl, or C₂₋₆alkynyl, wherein said alkyl, cycloalkyl, alkenyl, and alkynyl is optionally substituted with cyano, or one to three halogen;
each R⁶ is independently hydrogen, halogen, —CF₃, C₁₋₆alkyl, C₂₋₆alkylidene, —(CH₂)ₜ-cycloalkyl, —(CH₂)ₜ-heterocycloalkyl, —(CH₂)ₜ-aryl, or —(CH₂)ₜ-heteroaryl, —(CH₂)ₜ—OR⁷, —C(O)R⁷, —CN, or —N(R⁷)₂, wherein said —(CH₂)ₜ, —(CH₂)ₜ-cycloalkyl, —(CH₂)ₜ-aryl, —(CH₂)ₜ-heterocycloalkyl and —(CH₂)ₜ-heteroaryl substituent is optionally independently substituted with one to three alkyl, halogen, —CF₃ or —OR⁷;
each R⁷ is independently hydrogen, C₁₋₆alkyl, —CF₃, —SO₂R⁸, —N(R⁸)₂, —(CH₂)ₜ-cycloalkyl, —(CH₂)ₜ-heterocycloalkyl, —(CH₂)ₜ-aryl, or —(CH₂)ₜ-heteroaryl wherein said alkyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl is optionally substituted with C₁₋₆alkyl, C₂₋₆alkenyl, C₂₋₆alkynyl, halogen, —CF₃, or —OCF₃;
each R⁸ is hydrogen, C₁₋₆alkyl, or —(CH₂)ₜ-aryl, wherein said C₁₋₆alkyl or —(CH₂)ₜ-aryl is optionally substituted with one to three halogen; and
each t is an integer independently selected from 0, 1, 2, 3, and 4; or pharmaceutically acceptable salts thereof.

2. A compound of formula I: 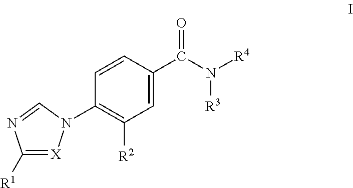
wherein
X is CH or N;
R¹ is hydrogen, C₁₋₆alkyl, C₃₋₆cycloalkyl, or C₂₋₆alkenyl, wherein said alkyl, cycloalkyl and alkenyl is optionally substituted with one to three halogen;
R² is hydrogen, —CF₃, cyano, halogen, C₁₋₆alkyl, or —OR⁵;
R³ and R⁴ are each independently hydrogen, C₁₋₆alkyl, C₂₋₆alkenyl, —(CH₂)ₜ-cycloalkyl, —(CH₂)ₜ-heterocycloalkyl, —(CH₂)ₜ-aryl, or —(CH₂)ₜ-heteroaryl, wherein said alkyl, alkenyl, —(CH₂)ₜ, —(CH₂)ₜ-cycloalkyl, —(CH₂)ₜ-heterocycloalkyl, —(CH₂)ₜ-aryl, or —(CH₂)ₜ-heteroaryl, R³ or R⁴ substituent is optionally substituted with one to three R⁶; or R³ and R⁴ together with the nitrogen they are bonded to form a heterocycloalkyl moiety, wherein said heterocycloalkyl moiety is optionally substituted with one to three R⁶;
R⁵ is hydrogen, C₁₋₆alkyl, C₃₋₆cycloalkyl, C₂₋₆alkenyl, or C₂₋₆alkynyl, wherein said alkyl, cycloalkyl, alkenyl, and alkynyl is optionally substituted with cyano, or one to three halogen;
each R⁶ is independently hydrogen, halogen, —CF₃, (C₁₋₆)alkyl, —(CH₂)ₜ-cycloalkyl, —(CH₂)ₜ-heterocycloalkyl, —(CH₂)ₜ-aryl, or —(CH₂)ₜ-heteroaryl, —OR⁷, —C(O)R⁷, —CN, or —N(R⁷)₂, wherein said —(CHA, —(CH₂)ₜ-cycloalkyl, —(CH₂)ₜ-heterocycloalkyl, —(CH₂)ₜ-aryl, or —(CH₂)ₜ-heteroaryl, substituent is optionally independently substituted with one to three alkyl, halogen, —CF₃ or —OR⁷;
each R⁷ is independently hydrogen, alkyl, —CF₃, —SO₂R⁸, —N(R⁸)₂, —(CH₂)ₜ-cycloalkyl, —(CH₂)ₜ-heterocycloalkyl, —(CH₂)ₜ-aryl, or —(CH₂)ₜ-heteroaryl wherein said alkyl, —(CH₂)ₜ-cycloalkyl, —(CH₂)ₜ-heterocycloalkyl, —(CH₂)ₜ-aryl, or —(CH₂)ₜ-heteroaryl is optionally substituted with C₁₋₆alkyl, C₂₋₆alkenyl, C₂₋₆alkynyl, halogen, —CF₃, or —OCF₃;
R⁸ is hydrogen, C₁₋₆alkyl, or —(CH₂)ₜ-aryl, wherein said alkyl or —(CH₂)ₜ-aryl is optionally substituted with one to three halogen; and
each t is an integer independently selected from 0, 1, 2, 3, and 4; or pharmaceutically acceptable salts thereof.

3. A compound according to claim 1 or 2 wherein X is CH.

4. A compound according to claim 3 wherein R³ is hydrogen and R⁴ is C₁₋₆alkyl, —(CH₂)ₜ-cycloalkyl, —(CH₂)ₜ-heterocycloalkyl, —CH₂)ₜ-aryl, or —(CH₂)ₜ-heteroaryl, wherein said C₁₋₆alkyl, —(CH₂), —(CH₂)ₜ-cycloalkyl, —(CH₂)ₜ-heterocycloalkyl, —(CH₂)ₜ-aryl, or —(CH₂)ₜ-heteroaryl R⁴ substituent is optionally substituted with one to three R⁶.

5. A compound according to claim 4 wherein R³ is hydrogen and R⁴ is C₁₋₆alkyl and said alkyl R⁴ substituent is optionally substituted with one to three R⁶.

6. A compound according to claim 5 wherein R³ is hydrogen and R⁴ is —(CH₂)ₜ-cycloalkyl and said —(CH₂)ₜ-cycloalkyl R⁴ substituent is optionally substituted with one to three R⁶.

7. A compound according to claim 6 wherein R³ is hydrogen and R⁴ is —(CH₂)ₜ-heterocycloalkyl and said —(CH₂)ₜ-heterocycloalkyl R⁴ substituent is optionally substituted with one to three R⁶.

8. A compound according to claim 7 wherein R³ is hydrogen and R⁴ is —(CH₂)ₜ-aryl and said —(CH₂)ₜ-aryl R⁴ substituent is optionally substituted with one to three R⁶.

9. A compound according to claim 8 wherein R³ is hydrogen and R⁴ is —(CH₂)ₜ-heteroaryl and said —(CH₂)ₜ-heteroaryl R⁴ substituent is optionally substituted with one to three R⁶.

10. A compound according to claim 9 wherein R³ and R⁴ together with the nitrogen they are bonded to form a heterocycloalkyl, wherein said heterocycloalkyl is optionally substituted with one to three R⁶.

11. A compound according to claim 10 wherein each R⁶ is independently —OR⁷ or —(CH₂)ₜ-aryl.

12. A compound according to claim 11 wherein R³ is hydrogen, C₁₋₆alkyl, or —(CH₂)ₜ-cycloalkyl wherein said C₁₋₆alkyl or —(CH₂)ₜ-cycloalkyl is optionally substituted with one to three halogen.

13. A compound according to claim 12 wherein R¹ is C₁₋₆alkyl.

14. A compound according to claim 13 wherein R¹ is methyl.

15. A compound according to claim 14 wherein R² is —OR⁵.

16. A compound according to claim 15 wherein R⁵ is hydrogen or C₁₋₆alkyl.

17. A compound according to claim 16 wherein R⁵ is methyl.

18. A compound according to claim 2 selected from the group consisting of:
N-[(5-chloro-1-benzothien-3-yl)methyl]-3-methoxy-4-(4-methyl-1H-imidazol-1-yl)benzamide;
1-[2-methoxy-4-({(3R)-3-[2-(trifluoromethyl)phenoxy]pyrrolidin-1-yl}carbonyl)phenyl]-4-methyl-1H-imidazole;
N-[2-(5-chloro-1H-indol-3-yl)ethyl]-3-methoxy-4-(4-methyl-1H-imidazol-1-yl)benzamide;
3-methoxy-4-(4-methyl-1H-imidazol-1-yl)-N-[(1-phenylcyclopentyl)methyl]benzamide;
3-methoxy-4-(4-methyl-1H-imidazol-1-yl)-N-({4-[4-(trifluoromethyl)phenyl]tetrahydro-2H-pyran-4-yl}methyl)benzamide;
N-(adamantan-2-ylmethyl)-3-methoxy-4-(4-methyl-1H-imidazol-1-yl)benzamide;
N-{[5-(4-chlorophenyl)-2-thienyl]methyl}-3-methoxy-4-(4-methyl-1H-imidazol-1-yl)benzamide;
3-methoxy-4-(4-methyl-1H-imidazol-1-yl)-N-({2-[4-(trifluoromethyl)phenyl]-1,3-thiazol-4-yl}methyl)benzamide;
3-methoxy-4-(4-methyl-1H-imidazol-1-yl)-N-{3-methyl-4-[3-(trifluoro-methyl)phenyl]-1H-pyrazol-5-yl}benzamide;
3-methoxy-4-(4-methyl-1H-imidazol-1-yl)-N-[(2-phenyl-1,3-thiazol-5-yl)methyl]benzamide;
N-[2-(5,7-dichloro-2-methyl-1H-indol-3-yl)ethyl]-3-methoxy-4-(4-methyl-1H-imidazol-1-yl)benzamide;
N-{[1-(4-chlorophenyl)cyclopentyl]methyl}-3-methoxy-4-(4-methyl-1H-imidazol-1-yl)benzamide;
N-{[1-(4-bromophenyl)cyclopropyl]methyl}-3-methoxy-4-(4-methyl-1H-imidazol-1-yl)benzamide;
N-{[1-(4-fluorophenyl)cyclobutyl]methyl}-3-methoxy-4-(4-methyl-1H-imidazol-1-yl)benzamide;
1-[2-methoxy-4-({(3S)-3-[2-(trifluoromethyl)phenoxy]pyrrolidin-1-yl}carbonyl)phenyl]-4-methyl-1H-imidazole;
N-[2-(5-bromo-1H-indol-3-yl)ethyl]-3-methoxy-4-(4-methyl-1H-imidazol-1-yl)benzamide;
3-methoxy-4-(4-methyl-1H-imidazol-1-yl)-N-({1-[3-(trifluoromethyl)phenyl]cyclobutyl}methyl)benzamide;
N-{[2-(4-chlorophenyl)-1,3-thiazol-4-yl]methyl}-3-methoxy-4-(4-methyl-1H-imidazol-1-yl)benzamide;
N-(3-chloro-4-fluorobenzyl)-3-methoxy-4-(4-methyl-1H-imidazol-1-yl)benzamide; or a pharmaceutically acceptable salt thereof.

19. A method for the treatment of a disease or condition selected from the group consisting of neurological and psychiatric disorders comprising administering to the mammal an effective amount of compound of claim 1 or pharmaceutically acceptable salt thereof.

20. A pharmaceutical composition comprising a compound of claim 1 or a pharmaceutically acceptable salt thereof and a pharmaceutically acceptable carrier.

21. The composition of claim 20 further comprising an atypical antipsychotic, a cholinesterase inhibitor, dimebon or NMDA receptor antagonist.
